SEQUENCE LISTING (53 sequences)

SEQ ID NO: 1 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 2 - protein, 132 aa, Homo sapiens
MSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 3 - protein, 483 aa, Homo sapiens
MFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 4 - protein, 18 aa, Homo sapiens
MRVLLAALGLLFLGALRA

SEQ ID NO: 5 - protein, 367 aa, Homo sapiens
FPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAG

SEQ ID NO: 6 - protein, 21 aa, Homo sapiens
PVLFWVILVLVVVVGSSAFLL

SEQ ID NO: 7 - protein, 189 aa, Homo sapiens
CHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 8 - protein, 8 aa, Artificial Sequence
  synthetic construct
GYTFTTYT

SEQ ID NO: 9 - protein, 8 aa, Artificial Sequence
  synthetic construct
INPSSGCS

SEQ ID NO: 10 - protein, 16 aa, Artificial Sequence
  synthetic construct
ARRADYGNYEYTWFAY

SEQ ID NO: 11 - protein, 6 aa, Artificial Sequence
  synthetic construct
QNVGTN

SEQ ID NO: 12 - protein, 3 aa, Artificial Sequence
  synthetic construct
SAS

SEQ ID NO: 13 - protein, 9 aa, Artificial Sequence
  synthetic construct
QQYHTYPLT

SEQ ID NO: 14 - protein, 123 aa, Artificial Sequence
  synthetic construct
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSS

SEQ ID NO: 15 - protein, 107 aa, Artificial Sequence
  synthetic construct
VIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIK

SEQ ID NO: 16 - protein, 5 aa, Artificial Sequence
  synthetic construct
GGGGS

SEQ ID NO: 17 - protein, 15 aa, Artificial Sequence
  synthetic construct
SGGGSGGGGSGGGGS

SEQ ID NO: 18 - protein, 245 aa, Artificial Sequence
  synthetic construct
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIK

SEQ ID NO: 19 - protein, 151 aa, Artificial Sequence
  synthetic construct
ATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDC

SEQ ID NO: 20 - protein, 24 aa, Homo sapiens
FWVLVVVGGVLACYSLLVTVAFII

SEQ ID NO: 21 - protein, 21 aa, Homo sapiens
LCYLLDGILFIYGVILTALFL

SEQ ID NO: 22 - protein, 28 aa, Homo sapiens
IYIWAPLAGTCGVLLLSLVITLYCNHRN

SEQ ID NO: 23 - protein, 4 aa, Artificial Sequence
  synthetic constructs
  misc_feature (2)..(3): Xaa can be any naturally occurring amino acid
  MISC_FEATURE (4)..(4): Xaa is L or I
YXXX

SEQ ID NO: 24 - protein, 16 aa, Artificial Sequence
  synthetic construct
  misc_feature (2)..(3): Xaa can be any naturally occurring amino acid
  MISC_FEATURE (4)..(4): Xaa is L or I
  misc_feature (5)..(9): Xaa can be any naturally occurring amino acid
  MISC_FEATURE (10)..(12): Xaa is absent or any naturally occurring amino acid
  misc_feature (14)..(15): Xaa can be any naturally occurring amino acid
  MISC_FEATURE (16)..(16): Xaa is L or I
YXXXXXXXXXXXYXXX

SEQ ID NO: 25 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 44 aa, Homo sapiens
FWVRSKRSRLLHSDYMNMTPRRPGPTRKHYQPYAPPRDFAAYRS

SEQ ID NO: 27 - protein, 44 aa, Homo sapiens
FWVRSKRSRLLHSDYMNMTPRRPGPTRKHYQAYAAARDFAAYRS

SEQ ID NO: 28 - protein, 156 aa, Artificial Sequence
  synthetic construct
FWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 12 aa, Homo sapiens
EPKSCDKTHTCP

SEQ ID NO: 30 - protein, 12 aa, Homo sapiens
EPKSPDKTHTCP

SEQ ID NO: 31 - protein, 221 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKV

SEQ ID NO: 32 - protein, 223 aa, Homo sapiens
PCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 33 - protein, 232 aa, Artificial Sequence
  synthetic construct
EPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 20 aa, Artificial Sequence
  synthetic construct
MDFQVQIFSFLLISASVIMS

SEQ ID NO: 35 - protein, 666 aa, Artificial Sequence
  synthetic construct
QVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 689 aa, Artificial Sequence
  synthetic construct
MDFQVQIFSFLLISASVIMSRMAQVQLQQSGAELARPGASVKMSCKASGYTFTTYTIHWVRRRPGHDLEWIGYINPSSGCSDYNQNFKGKTTLTADKSSNTAYMQLNSLTSEDSAVYYCARRADYGNYEYTWFAYWGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 8 aa, Artificial Sequence
  synthetic construct
GVSLPDYG

SEQ ID NO: 38 - protein, 7 aa, Artificial Sequence
  synthetic construct
IWGSETT

SEQ ID NO: 39 - protein, 14 aa, Artificial Sequence
  synthetic construct
AKHYYYGGSYAMDY

SEQ ID NO: 40 - protein, 6 aa, Artificial Sequence
  synthetic construct
QDISKY

SEQ ID NO: 41 - protein, 3 aa, Artificial Sequence
  synthetic construct
HTS

SEQ ID NO: 42 - protein, 9 aa, Artificial Sequence
  synthetic construct
QQGNTLPYT

SEQ ID NO: 43 - protein, 120 aa, Artificial Sequence
  synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 107 aa, Artificial Sequence
  synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 45 - protein, 15 aa, Artificial Sequence
  synthetic construct
GGGGSGGGGSGGGGS

SEQ ID NO: 46 - protein, 263 aa, Artificial Sequence
  synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 47 - protein, 15 aa, Artificial Sequence
  synthetic construct
ESKYGPPCPPCPDPK

SEQ ID NO: 48 - protein, 29 aa, Homo sapiens
FWVLVVVGGVLACYSLLVTVAFIIFWVRS

SEQ ID NO: 49 - protein, 42 aa, Homo sapiens
KRGRKKLLYIFKQPFMRPVQTTQEEDGCSCRFPEEEEGGCEL

SEQ ID NO: 50 - protein, 154 aa, Artificial Sequence
  synthetic onstruct
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 21 aa, Artificial Sequence
  synthetic construct
MALPVTALLLPLALLLHAARP

SEQ ID NO: 52 - protein, 440 aa, Artificial Sequence
  synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPDPKFWVLVVVGGVLACYSLLVTVAFIIFWVRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 461 aa, Artificial Sequence
  synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPDPKFWVLVVVGGVLACYSLLVTVAFIIFWVRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2023/0172986 A1
(43) Pub. Date: Jun. 8, 2023

(54) TREATMENT AND PREVENTION OF ALLOREACTIVITY USING VIRUS-SPECIFIC IMMUNE CELLS EXPRESSING CHIMERIC ANTIGEN RECEPTORS

(71) Applicant: Baylor College of Medicine, Houston, TX (US)

(72) Inventors: David H. Quach, Bellaire, TX (US); Cliona M. Rooney, Houston, TX (US); Carlos A. Ramos, Houston, TX (US)

(21) Appl. No.: 17/997,171

(22) PCT Filed: Apr. 27, 2021
(86) PCT No.: PCT/US2021/070461

§ 371 Date: Oct. 26, 2022

Related U.S. Application Data
(60) Provisional application No. 63/015,769, filed on Apr. 27, 2020.

(51) Int. Cl. A61K 35/17; A61P 35/00; C07K 14/725; C07K 14/705; C07K 16/28

(52) U.S. Cl. CPC A61K 35/17; A61P 35/00; C07K 14/7051; C07K 14/70521; C07K 16/2878

(57) ABSTRACT

Treatment and prevention of diseases/conditions characterised by an alloreactive immune response using virus-specific immune cells, comprising a chimeric antigen receptor (CAR) or nucleic acid encoding a CAR, wherein the CAR comprises: (i) an antigen-binding domain which binds specifically to CD30, (ii) a transmembrane domain, and (iii) a signalling domain, wherein the signalling domain comprises: (a) an amino acid sequence derived from the intracellular domain of CD28, and (b) an amino acid sequence comprising an immunoreceptor tyrosine-based activation motif (ITAM), is disclosed.

This application claims priority from U.S. 63/015,769 filed 27 Apr. 2020, the contents and elements of which are herein incorporated by reference for all purposes.

### TECHNICAL FIELD

The present invention relates to molecular and cell biology, and also relates to methods of medical treatment and prophylaxis.

### BACKGROUND

Despite the success of autologous chimeric antigen receptor (CAR) T cells in hematologic malignancies, barriers to more widespread use of this potentially curative therapy exist. Manufacture failure, disease progression prior to infusion, and exorbitant cost prove prohibitive for many. There is an urgent need for an immediately available CAR T cell option.

“Off-the-shelf” T cell products derived from healthy donors that can rapidly be administered, would improve accessibility and reduce the cost of adoptive cellular immunotherapy. However, the development of “off-the-shelf” CAR T cell therapies has been hindered by two major pitfalls: potential for polyclonally activated CAR T cells from unrelated donors to cause graft versus host disease (GVHD), and rejection of allogeneic CAR T cells by recipient alloreactive T cells.

### SUMMARY

In a first aspect, the present disclosure provides a virus-specific immune cell, comprising a chimeric antigen receptor (CAR) or nucleic acid encoding a CAR, wherein the CAR comprises: (I) an antigen-binding domain which binds specifically to CD30, (ii) a transmembrane domain, and (iii) a signalling domain, wherein the signalling domain comprises: (a) an amino acid sequence derived from the intracellular domain of CD28, and (b) an amino acid sequence comprising an immunoreceptor tyrosine-based activation motif (ITAM).

In some embodiments, the signalling domain comprises an amino acid sequence having at least 80% amino acid sequence identity to SEQ ID NO:26.

In some embodiments, the transmembrane domain is derived from the transmembrane domain of CD28.

In some embodiments, the transmembrane domain comprises an amino acid sequence having at least 80% amino acid sequence identity to SEQ ID NO:20.

In some embodiments, the antigen-binding domain comprises an amino acid sequence having at least 80% amino acid sequence identity to SEQ ID NO:14, and an amino acid sequence having at least 80% amino acid sequence identity to SEQ ID NO:15.

In some embodiments, the antigen-binding domain comprises an amino acid sequence having at least 80% amino acid sequence identity to SEQ ID NO:18.

In some embodiments, the signalling domain comprises: (a) an amino acid sequence derived from the intracellular domain of CD3ζ.

In some embodiments, the signalling domain comprises an amino acid sequence having at least 80% amino acid sequence identity to SEQ ID NO:25.

In some embodiments, the CAR additionally comprises a hinge region provided between the antigen-binding domain and the transmembrane domain.

In some embodiments, the hinge region comprises an amino acid sequence having at least 80% amino acid sequence identity to SEQ ID NO:33.

In some embodiments, the CAR comprises an amino acid sequence having at least 80% amino acid sequence identity to SEQ ID NO:35 or 36.

In some embodiments, the virus-specific immune cell comprises a CAR comprising an antigen-binding domain which binds specifically to a target antigen other than CD30, or nucleic acid encoding a CAR comprising an antigen-binding domain which binds specifically to a target antigen other than CD30.

The present disclosure also provides a virus-specific immune cell, comprising: a chimeric antigen receptor (CAR) or nucleic acid encoding a CAR, wherein the CAR comprises: (i) an antigen-binding domain which binds specifically to CD30, (ii) a transmembrane domain, and (iii) a signalling domain comprising an immunoreceptor tyrosine-based activation motif (ITAM);
wherein the virus-specific immune cell comprises a CAR comprising an antigen-binding domain which binds specifically to a target antigen other than CD30, or nucleic acid encoding a CAR comprising an antigen-binding domain which binds specifically to a target antigen other than CD30.

In some embodiments, the virus-specific immune cell comprises more than one non-identical CAR, or nucleic acid encoding more than one non-identical CAR.

In some embodiments, the target antigen other than CD30 is a cancer cell antigen.

In some embodiments, the target antigen other than CD30 is selected from CD19, CD20, CD22, ROR1R, CD4, CD7, CD38, BCMA, Mesothelin, EGFR, GPC3, MUC1, HER2, GD2, CEA, EpCAM, LeY and PSCA.

In some embodiments, the target antigen other than CD30 is CD19.

In some embodiments, the virus-specific immune cell is a virus-specific T cell.

In some embodiments, the virus-specific immune cell is specific for Epstein-Barr virus (EBV).

The present disclosure also provides a method for producing a virus-specific immune cell, comprising:
modifying a virus-specific immune cell to comprise a chimeric antigen receptor (CAR) or nucleic acid encoding a CAR, wherein the CAR comprises: (i) an antigen-binding domain which binds specifically to CD30, (ii) a transmembrane domain, and (iii) a signalling domain, wherein the signalling domain comprises: (a) an amino acid sequence derived from the intracellular domain of CD28, and (b) an amino acid sequence comprising an immunoreceptor tyrosine-based activation motif (ITAM).

In some embodiments, the signalling domain comprises an amino acid sequence having at least 80% amino acid sequence identity to SEQ ID NO:26.

In some embodiments, the transmembrane domain is derived from the transmembrane domain of CD28.

In some embodiments, the transmembrane domain comprises an amino acid sequence having at least 80% amino acid sequence identity to SEQ ID NO:20.

In some embodiments, the antigen-binding domain comprises an amino acid sequence having at least 80% amino acid sequence identity to SEQ ID NO:14, and an amino acid sequence having at least 80% amino acid sequence identity to SEQ ID NO:15.

In some embodiments, the antigen-binding domain comprises an amino acid sequence having at least 80% amino acid sequence identity to SEQ ID NO:18.

In some embodiments, the signalling domain comprises: (a) an amino acid sequence derived from the intracellular domain of CD3ζ.

In some embodiments, the signalling domain comprises an amino acid sequence having at least 80% amino acid sequence identity to SEQ ID NO:25.

In some embodiments, the CAR additionally comprises a hinge region provided between the antigen-binding domain and the transmembrane domain.

In some embodiments, the hinge region comprises an amino acid sequence having at least 80% amino acid sequence identity to SEQ ID NO:33.

In some embodiments, the CAR comprises an amino acid sequence having at least 80% amino acid sequence identity to SEQ ID NO:35 or 36.

In some embodiments, the virus-specific immune cell comprises a CAR comprising an antigen-binding domain which binds specifically to a target antigen other than CD30, or nucleic acid encoding a CAR comprising an antigen-binding domain which binds specifically to a target antigen other than CD30.

The present invention also provides a method for producing a virus-specific immune cell, comprising:
modifying a virus-specific immune cell to comprise a chimeric antigen receptor (CAR) or nucleic acid encoding a CAR, wherein the CAR comprises: (i) an antigen-binding domain which binds specifically to CD30, (ii) a transmembrane domain, and (iii) a signalling domain comprising an immunoreceptor tyrosine-based activation motif (ITAM);
wherein the virus-specific immune cell comprises a CAR comprising an antigen-binding domain which binds specifically to a target antigen other than CD30, or nucleic acid encoding a CAR comprising an antigen-binding domain which binds specifically to a target antigen other than CD30.

In some embodiments, the method further comprises:
modifying a virus-specific immune cell to comprise a chimeric antigen receptor (CAR) or nucleic acid encoding a CAR, wherein the CAR comprises an antigen-binding domain which binds specifically to a target antigen other than CD30.

In some embodiments, the virus-specific immune cell comprises more than one non-identical CAR, or nucleic acid encoding more than one non-identical CAR.

In some embodiments, the target antigen other than CD30 is a cancer cell antigen.

In some embodiments, the target antigen other than CD30 is selected from CD19, CD20, CD22, ROR1R, CD4, CD7, CD38, BCMA, Mesothelin, EGFR, GPC3, MUC1, HER2, GD2, CEA, EpCAM, LeY and PSCA.

In some embodiments, the target antigen other than CD30 is CD19.

In some embodiments, the virus-specific immune cell is a virus-specific T cell.

In some embodiments, the virus-specific immune cell is specific for Epstein-Barr virus (EBV).

The present disclosure also provides a virus-specific immune cell obtained or obtainable by the method according to the present disclosure.

The present disclosure also provides a pharmaceutical composition comprising a virus-specific immune cell according to the present disclosure and a pharmaceutically acceptable carrier, adjuvant, excipient or diluent.

The present disclosure also provides a virus-specific immune cell or a pharmaceutical composition according to the present disclosure, for use in a method of medical treatment or prophylaxis.

The present disclosure also provides a virus-specific immune cell or a pharmaceutical composition according to the present disclosure, for use in a method of treating or preventing a cancer.

The present disclosure also provides the use of a virus-specific immune cell or a pharmaceutical composition according to the present disclosure, in the manufacture of a medicament for treating or preventing a cancer.

The present disclosure also provides a method of treating or preventing a cancer comprising administering to a subject a therapeutically or prophylactically effective quantity of virus-specific immune cells or a pharmaceutical composition according to the present disclosure.

In some embodiments, the cancer is selected from the group consisting of: a CD30-positive cancer, an EBV-associated cancer, a hematological cancer, a myeloid hematologic malignancy, a hematopoietic malignancy, a lymphoblastic hematologic malignancy, myelodysplastic syndrome, leukemia, T cell leukemia, acute myeloid leukemia, chronic myeloid leukemia, acute lymphoblastic leukemia, lymphoma, Hodgkin's lymphoma, non-Hodgkin's lymphoma, B cell non-Hodgkin's lymphoma, diffuse large B cell lymphoma, primary mediastinal B cell lymphoma, EBV-associated lymphoma, EBV-positive B cell lymphoma, EBV-positive diffuse large B cell lymphoma, EBV-positive lymphoma associated with X-linked lymphoproliferative disorder, EBV-positive lymphoma associated with HIV infection/AIDS, oral hairy leukoplakia, Burkitt's lymphoma, post-transplant lymphoproliferative disease, central nervous system lymphoma, anaplastic large cell lymphoma, T cell lymphoma, ALK-positive anaplastic T cell lymphoma, ALK-negative anaplastic T cell lymphoma, peripheral T cell lymphoma, cutaneous T cell lymphoma, NK-T cell lymphoma, extra-nodal NK-T cell lymphoma, thymoma, multiple myeloma, a solid cancer, epithelial cell cancer, gastric cancer, gastric carcinoma, gastric adenocarcinoma, gastrointestinal adenocarcinoma, liver cancer, hepatocellular carcinoma, cholangiocarcinoma, head and neck cancer, head and neck squamous cell carcinoma, oral cavity cancer, oropharyngeal cancer, oropharyngeal carcinoma, oral cancer, laryngeal cancer, nasopharyngeal carcinoma, oesophageal cancer, colorectal cancer, colorectal carcinoma, colon cancer, colon carcinoma, cervical carcinoma, prostate cancer, lung cancer, non-small cell lung cancer, small cell lung cancer, lung adenocarcinoma, squamous lung cell carcinoma, bladder cancer, urothelial carcinoma, skin cancer, melanoma, advanced melanoma, renal cell cancer, renal cell carcinoma, ovarian cancer, ovarian carcinoma, mesothelioma, breast cancer, brain cancer, glioblastoma, prostate cancer, pancreatic cancer, mastocytosis, advanced systemic mastocytosis, germ cell tumor or testicular embryonal carcinoma.

The present disclosure also provides a virus-specific immune cell or a pharmaceutical composition according to the present disclosure, for use in a method of treating or preventing a disease or condition characterised by an alloreactive immune response.

The present disclosure also provides the use of a virus-specific immune cell or a pharmaceutical composition according to the present disclosure, in the manufacture of a medicament for treating or preventing a disease or condition characterised by an alloreactive immune response.

The present disclosure also provides a method of treating or preventing a disease or condition characterised by an alloreactive immune response, comprising administering to a subject a therapeutically or prophylactically effective quantity of virus-specific immune cells or a pharmaceutical composition according to the present disclosure.

In some embodiments, the disease or condition characterised by an alloreactive immune response is a disease or condition associated with allotransplantation.

In some embodiments, the disease or condition is graft versus host disease (GVHD).

In some embodiments, the disease or condition is graft rejection.

In some embodiments, the method comprises administering a therapeutically or prophylactically effective quantity of the virus-specific immune cells or pharmaceutical composition to a donor subject for an allotransplant prior to harvesting the allotransplant.

In some embodiments, the method comprises administering a therapeutically or prophylactically effective quantity of the virus-specific immune cells or pharmaceutical composition to a recipient subject for an allotransplant.

In some embodiments, the method comprises contacting an allotransplant with a therapeutically or prophylactically effective quantity of the virus-specific immune cells or composition.

The present disclosure also provides a virus-specific immune cell or a pharmaceutical composition according to the present disclosure, for use in a method of treating or preventing a disease or condition by allotransplantation.

The present disclosure also provides the use of a virus-specific immune cell or a pharmaceutical composition according to the present disclosure, in the manufacture of a medicament for treating or preventing a disease or condition by allotransplantation.

The present disclosure also provides a method of treating or preventing a disease or condition by allotransplantation, comprising administering to a subject a therapeutically or prophylactically effective quantity of virus-specific immune cells or a pharmaceutical composition according to the present disclosure.

In some embodiments, the method comprises administering a therapeutically or prophylactically effective quantity of the virus-specific immune cells or pharmaceutical composition to a donor subject for an allotransplant prior to harvesting the allotransplant.

In some embodiments, the method comprises administering a therapeutically or prophylactically effective quantity of the virus-specific immune cells or pharmaceutical composition to a recipient subject for an allotransplant.

In some embodiments, the method comprises contacting an allotransplant with a therapeutically or prophylactically effective quantity of the virus-specific immune cells or composition.

In some embodiments, the allotransplantation comprises adoptive transfer of allogeneic immune cells.

In some embodiments, the disease or condition is a T cell dysfunctional disorder, a cancer or an infectious disease.

The present disclosure also provides a method of killing alloreactive immune cells, comprising contacting alloreactive immune cells with a virus-specific immune cell or a pharmaceutical composition according to the present disclosure.

### DESCRIPTION

The inventors developed a CAR-modified virus specific T cell (CAR-VST) approach for the elimination of hematologic malignancies without causing GVHD, and avoiding allorejection.

The present disclosure provides a strategy to eliminate alloreactive T cells in order to protect allogeneic tissues including off-the-shelf cellular therapies from graft rejection, or to treat GVHD.

CD30 has been identified as a marker of alloreactive T cells, and so the inventors targeted them by engineering therapeutic T cells to express a chimeric antigen receptor (CAR) directed against CD30 (CD30.CAR). CD30.CAR expressing VSTs can be employed in methods using allogeneic therapies, for reducing alloreactive immune responses in the recipient subject.

Administering allogeneic T cells into HLA-mismatched recipients carries the risk of alloreactive immune responses such as GVHD since a proportion of the T cells will inherently possess alloreactivity. The inventors used virus specific T cells (VSTs) as the platform cells for expressing the CD30.CAR since they have been shown to rarely cause GVHD in allogeneic recipients, which is likely a result of their restricted TCR repertoire. In particular, Epstein-Barr Virus-specific T cells (EBVSTs) have been administered to more than 300 allogeneic recipients without any evidence of GVHD.

In addition, CD30.CAR expressing VSTs are themselves protected from rejection by alloreactive T cells in the recipient, and can therefore be directly be used as an off-the-shelf therapy, for example, for the treatment of CD30+ cancers.

Thus CD30.CAR VSTs will (i) eliminate the alloreactive T cells they elicit in allogeneic hosts, and (ii) persist for sufficient time and with the requisite activity to eliminate CD30-positive cancer, without causing GVHD.

The CD30.CAR expressing VSTs can also be engineered to target additional target antigens, e.g. through engineering to express CARs specific for additional target antigen(s) other than CD30. Such cells are useful as off-the-shelf therapy for the treatment e.g. of cancers expressing the relevant target antigen(s), as they are able to kill cells expressing the target antigen(s), and are also able to eliminate CD30-expressing allogeneic T cells.

Virus-Specific Immune Cells

The present disclosure concerns virus-specific immune cells, in particular Epstein-Barr virus (EBV)-specific immune cells. It will be appreciated that where cells are referred to herein in the singular (i.e. “a/the cell”), pluralities/populations of such cells are also contemplated.

A ‘virus-specific immune cell’ as used herein refers to an immune cell which is specific for a virus. A virus-specific immune cell expresses/comprises a receptor (preferably a T cell receptor) capable of recognising a peptide of an antigen of a virus (e.g. when presented by an MHC molecule). The virus-specific immune cell may express/comprise such a receptor as a result of expression of endogenous nucleic acid encoding such antigen receptor, or as a result of having been engineered to express such a receptor. The virus-specific immune cell preferably expresses/comprises a TCR specific for a peptide of an antigen of a virus.

The immune cell may be a cell of hematopoietic origin, e.g. a neutrophil, eosinophil, basophil, dendritic cell, lymphocyte, or monocyte. A lymphocyte may be e.g. a T cell, B cell, NK cell, NKT cell or innate lymphoid cell (ILC), or a precursor thereof. The immune cell may express e.g. CD3 polypeptides (e.g. CD3γ CD3ε CD3ζ or CD3δ), TCR polypeptides (TCRα or TCRβ), CD27, CD28, CD4 or CD8. In some embodiments, the immune cell is a T cell, e.g. a CD3+ T cell. In some embodiments, the T cell is a CD3+, CD4+ T cell. In some embodiments, the T cell is a CD3+, CD8+ T cell. In some embodiments, the T cell is a T helper cell (TM cell). In some embodiments, the T cell is a cytotoxic T cell (e.g. a cytotoxic T lymphocyte (CTL)).

A virus-specific T cell may display certain functional properties of a T cell in response to the viral antigen for which the T cell is specific, or in response a cell comprising/expressing the virus/antigen. In some embodiments, the properties are functional properties associated with effector T cells, e.g. cytotoxic T cells.

In some embodiments, a virus-specific T cell may display one or more of the following properties: cytotoxicity to a cell comprising/expressing the virus/the viral antigen for which the T cell is specific; proliferation, IFNγ expression, CD107a expression, IL-2 expression, TNFα expression, perforin expression, granzyme expression, granulysin expression, and/or FAS ligand (FASL) expression in response to stimulation with the virus/the viral antigen for which the T cell is specific, or in response to exposure to a cell comprising/expressing the virus/the viral antigen for which the T cell is specific.

Virus-specific T cells express/comprise a TCR capable of recognising a peptide of the viral antigen for which the T cell is specific when presented by the appropriate MHC molecule. Virus-specific T cells may be CD4+ T cells and/or CD8+ T cells.

The virus for which the virus-specific immune cell is specific may be any virus. For example, the virus may be a dsDNA virus (e.g. adenovirus, herpesvirus, poxvirus), ssRNA virus (e.g. parvovirus), dsRNA virus (e.g. reovirus), (+)ssRNA virus (e.g. picomavirus, togavirus), (−)ssRNA virus (e.g. orthomyxovirus, rhabdovirus), ssRNA-RT virus (e.g. retrovirus) or dsDNA-RT virus (e.g. hepadnavirus). In particular, the present disclosure contemplates viruses of the families adenoviridae, herpesviridae, papillomaviridae, polyomaviridae, poxviridae, hepadnaviridae, parvoviridae, astroviridae, caliciviridae, picomaviridae, coronaviridae, flaviviridae, togaviridae, hepeviridae, retroviridae, orthomyxoviridae, arenaviridae, bunyaviridae, filoviridae, paramyxoviridae, rhabdoviridae and reoviridae. In some embodiments the virus is selected from Epstein-Barr virus, adenovirus, Herpes simplex type 1 virus, Herpes simplex type 2 virus, Varicella-zoster virus, Human cytomegalovirus, Human herpesvirus type 8, Human papillomavirus, BK virus, JC virus, Smallpox, Hepatitis B virus, Parvovirus B19, Human Astrovirus, Norwalk virus, coxsackievirus, hepatitis A virus, poliovirus, rhinovirus, severe acute respiratory syndrome virus, Hepatitis C virus, yellow fever virus, dengue virus, West Nile virus, TBE virus, Rubella virus, Hepatitis E virus, Human immunodeficiency virus, influenza virus, lassa virus, Crimean-Congo hemorrhagic fever virus, Hantaan virus, ebola virus, Marburg virus, measles virus, mumps virus, parainfluenza virus, picomavirus, respiratory syncytial virus, rabies virus, hepatitis D virus, rotavirus, orbivirus, coltivirus, and banna virus.

In some embodiments, the virus is selected from Epstein-Barr virus (EBV), adenovirus, cytomegalovius (CMV), human papilloma virus (HPV), influenza virus, measles virus, hepatitis B virus (HBV), hepatitis C virus (HCV), human immunodeficiency virus (HIV), lymphocytic choriomeningitis virus (LCMV), or herpes simplex virus (HSV).

In some embodiments, the virus-specific immune cell may be specific for a peptide/polypeptide of a virus e.g. selected from Epstein-Barr virus (EBV), adenovirus, cytomegalovius (CMV), human papilloma virus (HPV), influenza virus, measles virus, hepatitis B virus (HBV), hepatitis C virus (HCV), human immunodeficiency virus (HIV), lymphocytic choriomeningitis virus (LCMV), or herpes simplex virus (HSV).

A T cell which is specific for an antigen of a virus may be referred to herein as a virus-specific T cell (VST). A T cell which is specific for an antigen of a particular virus may be described as being specific for the relevant virus; for example, a T cell which is specific for an antigen of EBV may be referred to as an EBV-specific T cell, or “EBVST”.

Accordingly, in some embodiments the virus-specific immune cell is an Epstein-Barr virus-specific T cell (EBVST), adenovirus-specific T cell (AdVST), cytomegalovius-specific T cell (CMVST), human papilloma virus (HPVST), influenza virus-specific T cell, measles virus-specific T cell, hepatitis B virus-specific T cell (HBVST), hepatitis C virus-specific T cell (HCVST), human immunodeficiency virus-specific T cell (HIVST), lymphocytic choriomeningitis virus-specific T cell (LCMVST), or herpes simplex virus-specific T cell (HSVST).

In some preferred embodiments, the virus-specific immune cell is specific for a peptide/polypeptide of an EBV antigen. In preferred embodiments the virus-specific immune cell is an Epstein-Barr virus-specific T cell (EBVST).

EBV virology is described e.g. in Stanfield and Luftiq, F1000Res. (2017) 6:386 and Odumade et al., Clin Microbiol Rev (2011) 24(1):193-209, both of which are hereby incorporated by reference in their entirety.

EBV infects epithelial cells via binding of viral protein BMFR2 to P1 integrins, and binding of viral protein gH/gL with integrins αvβ8 and αvβ8. EBV infects B cells through interaction of viral glycoprotein gp350 with CD21 and/or CD35, followed by interaction of viral gp42 with MHC class II. These interactions trigger fusion of the viral envelope with the cell membrane, allowing the virus to enter the cell. Once inside, the viral capsid dissolves and the viral genome is transported to the nucleus.

EBV has two modes of replication; latent and lytic. The latent cycle does not result in production of virions, and can take place in place B cells and epithelial cells. The EBV genomic circular DNA resides in the cell nucleus as an episome and is copied by the host cell's DNA polymerase. In latency, only a fraction of EBV's genes are expressed, in one of three different patterns known as latency programs, which produce distinct sets of viral proteins and RNAs. The latent cycle is described e.g. in Amon and Farrell, Reviews in Medical Virology (2004) 15(3): 149-56, which is hereby incorporated by reference in its entirety.

EBNA1 protein and non-coding RNA EBER are expressed in each of latency programs 1-111. Latency programs II and III further involve expression of EBNALP, LMP1, LMP2A and LMP2B proteins, and latency program III further involves expression of EBNA2, EBNA3A, EBNA3B and EBNA3C.

EBNA1 is multifunctional, and has roles in gene regulation, extrachromosomal replication, and maintenance of the EBV episomal genome through positive and negative regulation of viral promoters (Duellman et al., J Gen Virol. (2009); 90(Pt 9): 2251-2259). EBNA2 is involved in the regulation of latent viral transcription and contributes to the immortalization of cells infected with EBV (Kempkes and Ling, Curr Top Microbiol Immunol. (2015) 391:35-59). EBNA-LP is required for transformation of native B cells, and recruits transcription factors for viral replication (Szymula et al., PLoS Pathog. (2018); 14(2):e1006890). EBNA3A, 3B and 3C interact with RBPJ to influence gene expression, contributing to survival and growth of infected cells (Wang et al., J Virol. (2016) 90(6):2906-2919). LMP1 regulates expression of genes involved in B cell activation (Chang et al., J. Biomed. Sci. (2003) 10(5): 490-504). LMP2A and LMP2B inhibit normal B cell signal transduction by mimicking the activated B cell receptor (Portis and Longnecker, Oncogene (2004) 23(53): 8619-8628). EBERs form ribonucleoprotein complexes with host cell proteins, and are proposed to have roles in cell transformation.

The latent cycle can progress according to any of latency programs I to III in B cells, and usually progresses from III to II to I. Upon infection of a resting naïve B cell, EBV enters latency program III. Expression of latency III genes activates the B cell, which becomes a proliferating blast. EBV then typically progresses to latency II by restricting expression to a subset of genes, which cause differentiation of the blast to a memory B cell. Further restriction of gene expression causes EBV to enter latency I. EBNA1 expression allows EBV to replicate when the memory B cell divides. In epithelial cells, only latency II occurs.

In primary infection, EBV replicates in oropharyngeal epithelial cells and establishes Latency III, II, and I infections in B-lymphocytes. EBV latent infection of B-lymphocytes is necessary for virus persistence, subsequent replication in epithelial cells, and release of infectious virus into saliva. EBV Latency III and II infections of B-lymphocytes, Latency II infection of oral epithelial cells, and Latency II infection of NK- or T cell can result in malignancies, marked by uniform EBV genome presence and gene expression.

Latent EBV in B cells can be reactivated to switch to lytic replication. The lytic cycle results in the production of infectious virions and can take place in place B cells and epithelial cells, and is reviewed e.g. by Kenney in Chapter 25 of Arvin et al., Human Herpesviruses: Biology, Therapy and Immunoprophylaxis; Cambridge University Press (2007), which is hereby incorporated by reference in its entirety.

Lytic replication requires the EBV genome to be linear. The latent EBV genome is episomal, and so it must be linearised for lytic reactivation. In B cells, lytic replication normally only takes place after reactivation from latency.

Immediate-early lytic gene products such as BZFL1 and BRLF1 act as transactivators, enhancing their own expression, and the expression of later lytic cycle genes.

Early lytic gene products have roles in viral replication (e.g. EBV DNA polymerase catalytic component BALF5; DNA polymerase processivity factor BMRF1, DNA binding protein BALF2, helicase BBLF4, primase BSLF1, and primase-associated protein BBLF2/3) and deoxynucleotide metabolism (e.g. thymidine kinase BXLF1, dUTPase BORF2). Other early lytic gene products act transcription factors (e.g. BMRF1, BRRF1), have roles in RNA stability and processing (e.g. BMLF1), or are involved in immune evasion (e.g. BHRF1, which inhibits apoptosis).

Late lytic gene products are traditionally classed as those expressed after the onset of viral replication. They generally encode structural components of the virion such as nucleocapsid proteins, as well as glycoproteins which mediate EBV binding and fusion (e.g. gp350/220, gp85, gp42, gp25). Other late lytic gene products have roles in immune evasion; BCLF1 encodes a viral homologue of IL-10, and BALF1 encodes a protein with homology to the anti-apoptotic protein Bcl2.

An “EBV-specific immune cell” as used herein refers to an immune cell which is specific for Epstein-Barr virus (EBV). An EBV-specific immune cell expresses/comprises a receptor (preferably a T cell receptor) capable of recognising a peptide of an antigen of EBV (e.g. when presented by an MHC molecule). The EBV-specific immune cell preferably expresses/comprises a TCR specific for a peptide of an EBV antigen presented by MHC class I.

In some embodiments, the EBV-specific immune cell is a T cell, e.g. a CD3+ T cell. In some embodiments, the T cell is a CD3+, CD4+ T cell. In some embodiments, the T cell is a CD3+, CD8+ T cell. In some embodiments, the T cell is a T helper cell (T_{H} cell)). In some embodiments, the T cell is a cytotoxic T cell (e.g. a cytotoxic T lymphocyte (CTL)).

An EBV-specific T cell may display certain functional properties of a T cell in response to the EBV antigen for which the T cell is specific, or in response a cell comprising/expressing EBV (e.g. a cell infected with EBV) or the relevant EBV antigen. In some embodiments, the properties are functional properties associated with effector T cells, e.g. cytotoxic T lymphocytes (CTLs).

In some embodiments, an EBV-specific T cell may display one or more of the following properties: cytotoxicity to a cell comprising/expressing EBV/the EBV antigen for which the T cell is specific; proliferation, IFNγ expression, CD107a expression, IL-2 expression, TNFα expression, perforin expression, granzyme expression, granulysin expression, and/or FAS ligand (FASL) expression in response to stimulation with EBV/the EBV antigen for which the T cell is specific, or in response to exposure to a cell comprising/expressing EBV/the EBV antigen for which the T cell is specific.

EBV-specific T cells preferably express/comprise a TCR capable of recognising a peptide of the EBV antigen for which the T cell is specific when presented by the appropriate MHC molecule. EBV-specific T cells may be CD4+ T cells and/or CD8+ T cells.

An immune cell specific for EBV may be specific for any EBV antigen, e.g. an EBV antigen described herein. A population of immune cell specific for EBV, or a composition comprising a plurality of immune cells specific for EBV, may comprise immune cells specific for one or more EBV antigens.

In some embodiments, an EBV antigen is an EBV latent antigen, e.g. a type II latency antigen (e.g. EBNA1, EBNA-LP, LMP1, LMP2A, LMP2B, BARF1, EBNA2, EBNA3A, EBNA3B or EBNA3C), a type II latency antigen (e.g. EBNA1, EBNA-LP, LMP1, LMP2A, LMP2B or BARF1), or a type I latency antigen, (e.g. EBNA1 or BARF1). In some embodiments, an EBV antigen is an EBV lytic antigen, e.g. an immediate-early lytic antigen (e.g. BZLF1, BRLF1 or BMRF1), an early lytic antigen (e.g. BMLF1, BMRF1, BXLF1, BALF1, BALF2, BARF1, BGLF5, BHRF1, BNLF2A, BNLF2B, BHLF1, BLLF2, BKRF4, BMRF2, FU or EBNA1-FUK), or a late lytic antigen (e.g. BALF4, BILF1, BILF2, BNFR1, BVRF2, BALF3, BALF5, BDLF3 or gp350).

Chimeric Antigen Receptors

The present disclosure relates to virus-specific immune cells comprising/expressing chimeric antigen receptors (CARs).

Chimeric Antigen Receptors (CARs) are recombinant receptor molecules which provide both antigen-binding and T cell activating functions. CAR structure and engineering is reviewed, for example, in Dotti et al., Immunol Rev (2014) 257(1), which is hereby incorporated by reference in its entirety.

CARs comprise an antigen-binding domain linked via a transmembrane domain to a signalling domain. An optional hinge or spacer domain may provide separation between the antigen-binding domain and transmembrane domain, and may act as a flexible linker. When expressed by a cell, the antigen-binding domain is provided in the extracellular space, and the signalling domain is intracellular.

The antigen-binding domain mediates binding to the target antigen for which the CAR is specific. The antigen-binding domain of a CAR may be based on the antigen-binding region of an antibody which is specific for the antigen to which the CAR is targeted. For example, the antigen-binding domain of a CAR may comprise amino acid sequences for the complementarity-determining regions (CDRs) of an antibody which binds specifically to the target antigen. The antigen-binding domain of a CAR may comprise or consist of the light chain and heavy chain variable region amino acid sequences of an antibody which binds specifically to the target antigen. The antigen-binding domain may be provided as a single chain variable fragment (scFv) comprising the sequences of the light chain and heavy chain variable region amino acid sequences of an antibody. Antigen-binding domains of CARs may target antigens based on other protein:protein interactions, such as ligand:receptor binding; for example an IL-13Rα2-targeted CAR has been developed using an antigen-binding domain based on IL-13 (see e.g. Kahlon et al. 2004 Cancer Res 64(24): 9160-9166).

The transmembrane domain is provided between the antigen-binding domain and the signalling domain of the CAR. The transmembrane domain provides for anchoring the CAR to the cell membrane of a cell expressing a CAR, with the antigen-binding domain in the extracellular space, and signalling domain inside the cell. Transmembrane domains of CARs may be derived from transmembrane region sequences for cell membrane-bound proteins (e.g. CD28, CD8, etc.).

Throughout this specification, polypeptides, domains and amino acid sequences which are ‘derived from’ a reference polypeptide/domain/amino acid sequence have at least 60%, preferably one of 70%, 75%, 80%, 85%, 90%, 91%, 92%, 93%, 94%, 95%, 96%, 97%, 98%, 99% or 100% amino acid sequence identity to the amino acid sequence of the reference polypeptide/domain/amino acid sequence.

Polypeptides, domains and amino acid sequences which are ‘derived from’ a reference polypeptide/domain/amino acid sequence preferably retain the functional and/or structural properties of the reference polypeptide/domain/amino acid sequence.

By way of illustration, an amino acid sequence derived from the intracellular domain of CD28 may comprise an amino acid sequence having 60%, preferably one of 70%, 75%, 80%, 85%, 90%, 91%, 92%, 93%, 94%, 95%, 96%, 97%, 98%, 99% or 100% amino acid sequence identity to the intracellular domain of CD28, e.g. as shown in SEQ ID NO:26. Furthermore, an amino acid sequence derived from the intracellular domain of CD28 preferably retains the functional properties of the amino acid sequence of SEQ ID NO:26, i.e. the ability activate CD28-mediated signalling.

The amino acid sequence of a given polypeptide or domain thereof can be retrieved from, or determined from a nucleic acid sequence retrieved from, databases known to the person skilled in the art. Such databases include GenBank, EMBL and UniProt.

The signalling domain comprises amino acid sequences required for activation of immune cell function. The CAR signalling domains may comprise the amino acid sequence of the intracellular domain of CD3-ζ, which provides immunoreceptor tyrosine-based activation motifs (ITAMs) for phosphorylation and activation of the CAR-expressing cell. Signalling domains comprising sequences of other ITAM-containing proteins have also been employed in CARs, such as domains comprising the ITAM containing region of FcγRI (Haynes et al., 2001 J Immunol 166(1):182-187). CARs comprising a signalling domain derived from the intracellular domain of CD3-ζ are often referred to as first generation CARs.

The signalling domains of CARs typically also comprise the signalling domain of a costimulatory protein (e.g. CD28, 4-1BB etc.), for providing the costimulation signal necessary for enhancing immune cell activation and effector function. CARs having a signalling domain including additional costimulatory sequences are often referred to as second generation CARs. In some cases CARs are engineered to provide for costimulation of different intracellular signalling pathways. For example, CD28 costimulation preferentially activates the phosphatidylinositol 3-kinase (P13K) pathway, whereas 4-1BB costimulation triggers signalling is through TNF receptor associated factor (TRAF) adaptor proteins. Signalling domains of CARs therefore sometimes contain costimulatory sequences derived from signalling domains of more than one costimulatory molecule. CARs comprising a signalling domain with multiple costimulatory sequences are often referred to as third generation CARs.

An optional hinge or spacer region may provide separation between the antigen-binding domain and the transmembrane domain, and may act as a flexible linker. Such regions may be or comprise flexible domains allowing the binding moiety to orient in different directions, which may e.g. be derived from the CH1-CH2 hinge region of IgG.

Through engineering to express a CAR specific for a particular target antigen, immune cells (typically T cells, but also other immune cells such as NK cells) can be directed to kill cells expressing the target antigen. Binding of a CAR-expressing T cell (CAR-T cell) to the target antigen for which it is specific triggers intracellular signalling, and consequently activation of the T cell. The activated CAR-T cell is stimulated to divide and produce factors resulting in killing of the cell expressing the target antigen.

Antigen-Binding Domain

An “antigen-binding domain” refers to a domain which is capable of binding to a target antigen. The target antigen may e.g. be a peptide/polypeptide, glycoprotein, lipoprotein, glycan, glycolipid, lipid, or fragment thereof. Antigen-binding domains according to the present disclosure may be derived from an antibody/antibody fragment (e.g. Fv, scFv, Fab, single chain Fab (scFab), single domain antibodies (e.g. VhH), etc.) directed against the target antigen, or another target antigen-binding molecule (e.g. a target antigen-binding peptide or nucleic acid aptamer, ligand or other molecule).

In some embodiments, the antigen-binding domain comprises an antibody heavy chain variable region (VH) and an antibody light chain variable region (VL) of an antibody capable of specific binding to the target antigen. In some embodiments, the domain capable of binding to a target antigen comprises or consists of an antigen-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)), affilin, armadillo repeat protein (ArmRP), OBody or fibronectin—reviewed e.g. in Reverdatto et al., Curr Top Med Chem. 2015; 15(12): 1082-1101, which is hereby incorporated by reference in its entirety (see also e.g. Boersma et al., J Biol Chem (2011) 286:41273-85 and Emanuel et al., Mabs (2011) 3:38-48).

The antigen-binding domains of the present disclosure generally comprise a VH and a VL of an antibody capable of specific binding to the target antigen. Antibodies generally comprise six complementarity-determining regions CDRs; three in the heavy chain variable region (VH): HC-CDR1, HC-CDR2 and HC-CDR3, and three in the light chain variable region (VL): LC-CDR1, LC-CDR2, and LC-CDR3. The six CDRs together define the paratope of the antibody, which is the part of the antibody which binds to the target antigen. The VH region and VL region comprise framework regions (FRs) either side of each CDR, which provide a scaffold for the CDRs. From N-terminus to C-terminus, VHs comprise the following structure: N term-[HC-FR1]-[HC-CDR1]-[HC-FR2]-[HC-CDR2]-[HC-FR3]-[HC-CDR3]-[HC-FR4]-C term; and VLs comprise the following structure: N term-[LC-FR1]-[LC-CDR1]-[LC-FR2]-[LC-CDR2]-[LC-FR3]-[LC-CDR3]-[LC-FR4]-C term.

VH and VL sequences may be provided in any suitable format provided that the antigen-binding domain can be linked to the other domains of the CAR. Formats contemplated in connection with the antigen-binding domain of the present disclosure include those described in Carter, Nat. Rev. Immunol (2006), 6: 343-357, such as scFv, dsFV, (scFv)₂ diabody, triabody, tetrabody, Fab, minibody, and F(ab)₂ formats.

In some embodiments, the antigen-binding domain comprises the CDRs of an antibody/antibody fragment which is capable of binding to the target antigen. In some embodiments, the antigen-binding domain comprises the VH region and the VL region of an antibody/antibody fragment which is capable of binding to the target antigen. A moiety comprised of the VH and a VL of an antibody may also be referred to herein as a variable fragment (Fv). The VH and VL may be provided on the same polypeptide chain, and joined via a linker sequence; such moieties are referred to as single-chain variable fragments (scFvs). Suitable linker sequences for the preparation of scFv are known to the skilled person, and may comprise serine and glycine residues.

In some embodiments, the antigen-binding domain comprises, or consists of, Fv capable of binding to the target antigen. In some embodiments, the antigen-binding domain comprises, or consists of, a scFv capable of binding to the target antigen.

The target antigen for which the antigen-binding domain (and thus the CAR) is specific may be any target antigen. In some embodiments, the target antigen is an antigen whose expression/activity, or whose upregulated expression/activity, is positively associated with a disease or disorder (e.g. a cancer, an infectious disease or an autoimmune disease). The target antigen is preferably expressed at the cell surface of a cell expressing the target antigen. It will be appreciated that the CAR directs effector activity of the cell expressing the CAR against cells/tissues expressing the target antigen for which the CAR comprises a specific antigen-binding domain.

In some embodiments, a target antigen may be a cancer cell antigen. A cancer cell antigen is an antigen which is expressed or over-expressed by a cancer cell. A cancer cell antigen may be any peptide/polypeptide, glycoprotein, lipoprotein, glycan, glycolipid, lipid, or fragment thereof. A cancer cell antigen's expression may be associated with a cancer. A cancer cell antigen may be abnormally expressed by a cancer cell (e.g. the cancer cell antigen may be expressed with abnormal localisation), or may be expressed with an abnormal structure by a cancer cell. A cancer cell antigen may be capable of eliciting an immune response. In some embodiments, the antigen is expressed at the cell surface of the cancer cell (i.e. the cancer cell antigen is a cancer cell surface antigen). In some embodiments, the part of the antigen which is bound by the antigen-binding molecule described herein is displayed on the external surface of the cancer cell (i.e. is extracellular). The cancer cell antigen may be a cancer-associated antigen. In some embodiments the cancer cell antigen is an antigen whose expression is associated with the development, progression or severity of symptoms of a cancer. The cancer-associated antigen may be associated with the cause or pathology of the cancer, or may be expressed abnormally as a consequence of the cancer. In some embodiments, the cancer cell antigen is an antigen whose expression is upregulated (e.g. at the RNA and/or protein level) by cells of a cancer, e.g. as compared to the level of expression of by comparable non-cancerous cells (e.g. non-cancerous cells derived from the same tissue/cell type). In some embodiments, the cancer-associated antigen may be preferentially expressed by cancerous cells, and not expressed by comparable non-cancerous cells (e.g. non-cancerous cells derived from the same tissue/cell type). In some embodiments, the cancer-associated antigen may be the product of a mutated oncogene or mutated tumor suppressor gene. In some embodiments, the cancer-associated antigen may be the product of an overexpressed cellular protein, a cancer antigen produced by an oncogenic virus, an oncofetal antigen, or a cell surface glycolipid or glycoprotein.

Cancer cell antigens are reviewed by Zarour H M, DeLeo A, Finn O J, et al. Categories of Tumor Antigens. In: Kufe D W, Pollock R E, Weichselbaum R R, et al., editors. Holland-Frei Cancer Medicine. 6th edition. Hamilton (ON): BC Decker; 2003. Cancer cell antigens include oncofetal antigens: CEA, Immature laminin receptor, TAG-72; oncoviral antigens such as HPV E6 and E7; overexpressed proteins: BING-4, calcium-activated chloride channel 2, cyclin-B1, 9D7, Ep-CAM, EphA3, HER2/neu, telomerase, mesothelin, SAP-1, survivin; cancer-testis antigens: BAGE, CAGE, GAGE, MAGE, SAGE, XAGE, CT9, CT10, NY-ESO-1, PRAME, SSX-2; lineage restricted antigens: MART1, Gp100, tyrosinase, TRP-1/2, MC1R, prostate specific antigen; mutated antigens: 1-catenin, BRCA1/2, CDK4, CML66, Fibronectin, MART-2, p53, Ras, TGF-βRII; post-translationally altered antigens: MUC1, idiotypic antigens: Ig, TCR. Other cancer cell antigens include heat-shock protein 70 (HSP70), heat-shock protein 90 (HSP90), glucose-regulated protein 78 (GRP78), vimentin, nucleolin, feto-acinar pancreatic protein (FAPP), alkaline phosphatase placental-like 2 (ALPPL-2), siglec-5, stress-induced phosphoprotein 1 (STIP1), protein tyrosine kinase 7 (PTK7), and cyclophilin B.

In some embodiments the cancer cell antigen is a cancer cell antigen described in Zhao and Cao, Front Immunol. (2019) 10: 2250, which is hereby incorporated by reference in its entirety. In some embodiments, a cancer cell antigen is selected from CD30, CD19, CD20, CD22, ROR1R, CD4, CD7, CD38, BCMA, Mesothelin, EGFR, GPC3, MUC1, HER2, GD2, CEA, EpCAM, LeY and PSCA.

In some embodiments, a cancer cell antigen is an antigen expressed by cells of a hematological malignancy. In some embodiments, a cancer cell antigen is selected from CD30, CD19, CD20, CD22, ROR1R, CD4, CD7, CD38 and BCMA.

In some embodiments, a cancer cell antigen is an antigen expressed by cells of a solid tumor. In some embodiments, a cancer cell antigen is selected from Mesothelin, EGFR, GPC3, MUC1, HER2, GD2, CEA, EpCAM, LeY and PSCA.

In some embodiments the cancer cell antigen is CD19. CD19 is a marker of B cells, and is a useful target for the treatment of e.g. B cell lymphomas, acute lymphoblastic leukemia (ALL), and chronic lymphocytic leukemia (CLL)—see e.g. Wang et al., Exp Hematol Oncol. (2012) 1:36.

In some embodiments, the antigen-binding domain (and thus the CAR) is multispecific. By “multispecific” it is meant that the antigen-binding domain displays specific binding to more than one target. In some embodiments the antigen-binding domain is a bispecific antigen-binding domain. In some embodiments the antigen-binding molecule comprises at least two different antigen-binding moieties (i.e. at least two antigen-binding moieties, e.g. comprising non-identical VHs and VLs). Individual antigen-binding moieties of multispecific antigen-binding domains may be connected, e.g. via linker sequences.

In some embodiments the antigen-binding domain binds to at least two, non-identical target antigens, and so is at least bispecific. The term “bispecific” means that the antigen-binding domain is able to bind specifically to at least two distinct antigenic determinants. In some embodiments, at least one of the target antigens for the multispecific antigen-binding domain/CAR is CD30.

Each of the target antigens may independently be a target antigen as described herein. In some embodiments each target antigen is independently a cancer cell antigen as described herein.

It will be appreciated that an antigen-binding domain according to the present disclosure (e.g. a multispecific antigen-binding domain) comprises antigen-binding moieties capable of binding to the target(s) for which the antigen-binding domain is specific. For example, an antigen-binding domain which is capable of binding to CD30 and an antigen other than CD30 may comprise: (i) an antigen-binding moiety which is capable of binding to CD30, and (ii) an antigen-binding moiety which is capable of binding to a target antigen other than CD30.

In aspects and embodiments of the present disclosure, the target antigen is CD30. Accordingly, in some aspects and embodiments of the present disclosure the antigen-binding domain is a CD30-binding domain.

CD30 (also known as TNFRSF8) is the protein identified by UniProt: P28908. CD30 is a single pass, type I transmembrane glycoprotein of the tumor necrosis factor receptor superfamily. CD30 structure and function is described e.g. in van der Weyden et al., Blood Cancer Journal (2017) 7: e603 and Muta and Podack Immunol. Res. (2013) 57(1-3):151-8, both of which are hereby incorporated by reference in their entirety.

Alternative splicing of mRNA encoded by the human TNFRSF8 gene yields three isoforms: isoform 1 (‘long’ isoform; UniProt: P28908-1, v1; SEQ ID NO:1), isoform 2 (‘cytoplasmic’, ‘short’ or ‘C30V’ isoform, UniProt: P28908-2; SEQ ID NO:2) in which the amino acid sequence corresponding to positions 1 to 463 of SEQ ID NO:1 are missing, and isoform 3 (UniProt: P28908-3; SEQ ID NO:3) in which the amino acid sequence corresponding to positions 1 to 111 and position 446 of SEQ ID NO:1 are missing. The N-terminal 18 amino acids of SEQ ID NO:1 form a signal peptide (SEQ ID NO:4), which is followed by a 367 amino acid extracellular domain (positions 19 to 385 of SEQ ID NO:1, shown in SEQ ID NO:5), a 21 amino acid transmembrane domain (positions 386 to 406 of SEQ ID NO:1, shown in SEQ ID NO:6), and a 189 amino acid cytoplasmic domain (positions 407 to 595 of SEQ ID NO:1, shown in SEQ ID NO:7).

In this specification “CD30” refers to CD30 from any species and includes CD30 isoforms, fragments, variants or homologues from any species. As used herein, a “fragment”, “variant” or “homologue” of a reference protein may optionally be characterised as having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of the reference protein (e.g. a reference isoform). In some embodiments fragments, variants, isoforms and homologues of a reference protein may be characterised by ability to perform a function performed by the reference protein.

In some embodiments, the CD30 is from a mammal (e.g. a primate (rhesus, cynomolgous, or human) and/or a rodent (e.g. rat or murine) CD30). In preferred embodiments the CD30 is a human CD30. Isoforms, fragments, variants or homologues may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature CD30 isoform from a given species, e.g. human. A fragment of CD30 may have a minimum length of one of 10, 20, 30, 40, 50, 100, 200, 300, 400, 500 or 590 amino acids, and may have a maximum length of one of 10, 20, 30, 40, 50, 100, 200, 300, 400, 500 or 595 amino acids.

In some embodiments, the CD30 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:1, 2 or 3.

In some embodiments, the CD30 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:5.

In some embodiments, a fragment of CD30 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:5 or 19.

The CD30-binding domain of the CAR of the present disclosure preferably displays specific binding to CD30 or a fragment thereof. The CD30-binding domain of the CAR of the present disclosure preferably displays specific binding to the extracellular domain of CD30. The CD30-binding domain may be derived from an anti-CD30 antibody or other CD30-binding agent, e.g. a CD30-binding peptide or CD30-binding small molecule.

The CD30-binding domain may be derived from the antigen-binding moiety of an anti-CD30 antibody.

Anti-CD30 antibodies include HRS3 and HRS4 (described e.g. in Hombach et al., Scand J Immunol (1998) 48(5):497-501), HRS3 derivatives described in Schlapschy et al., Protein Engineering, Design and Selection (2004) 17(12): 847-860, BerH2 (MBL Intemational Cat #K0145-3, RRID:AB_590975), SGN-30 (also known as cAC10, described e.g. in Forero-Torres et al., Br J Haematol (2009) 146:171-9), MDX-060 (described e.g. in Ansell et al., J Clin Oncol (2007) 25:2764-9; also known as 5F11, iratumumab), and MDX-1401 (described e.g. in Cardarelli et al., Clin Cancer Res. (2009) 15(10):3376-83), and anti-CD30 antibodies described in VWD 2020/068764 A1, WO 2003/059282 A2, WO 2006/089232 A2, VWD 2007/084672 A2, WYD 2007/044616 A2, WD 2005/001038 A2, US 2007/166309 A1, US 2007/258987 A1, WO 2004/010957 A2 and US 2005/009769 A1.

In some embodiments a CD30-binding domain according to the present disclosure comprises the CDRs of an anti-CD30 antibody. In some embodiments a CD30-binding domain according to the present disclosure comprises the VH and VL regions of an anti-CD30 antibody. In some embodiments a CD30-binding domain according to the present disclosure comprises a scFv comprising the VH and VL regions of an anti-CD30 antibody.

There are several different conventions for defining antibody CDRs and FRs, such as those described in Kabat et al., Sequences of Proteins of Immunological Interest, 5th Ed. Public Health Service, National Institutes of Health, Bethesda, Md. (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibodies described herein are defined according to VBASE2.

In some embodiments the antigen-binding domain of the present disclosure comprises:
a VH incorporating the following CDRs:
HC-CDR1 having the amino acid sequence of SEQ ID NO:8
HC-CDR2 having the amino acid sequence of SEQ ID NO:9
HC-CDR3 having the amino acid sequence of SEQ ID NO:10,
or a variant thereof in which one or two or three amino acids in one or more of HC-CDR1, HC-CDR2, or HC-CDR3 are substituted with another amino acid;
   and
a VL incorporating the following CDRs:
LC-CDR1 having the amino acid sequence of SEQ ID NO:11
LC-CDR2 having the amino acid sequence of SEQ ID NO:12
LC-CDR3 having the amino acid sequence of SEQ ID NO:13,
or a variant thereof in which one or two or three amino acids in one or more of LC-CDR1, LC-CDR2, or LC-CDR3 are substituted with another amino acid.

In some embodiments the antigen-binding domain comprises:
a VH comprising, or consisting of, an amino acid sequence having at least 80% sequence identity (e.g. at least 85%, 86%, 87%, 88%, 89%, 90%, 91%, 92%, 93%, 94%, 95%, 96%, 97%, 98%, 99% or 100%) to the amino acid sequence of SEQ ID NO:14;
   and
a VL comprising, or consisting of, an amino acid sequence having at least 80% sequence identity (e.g. at least 85%, 86%, 87%, 88%, 89%, 90%, 91%, 92%, 93%, 94%, 95%, 96%, 97%, 98%, 99% or 100%) to the amino acid sequence of SEQ ID NO:15.

In some embodiments, a CD30-binding domain may comprise or consist of a single chain variable fragment (scFv) comprising a VH sequence and a VL sequence as described herein. The VH sequence and VL sequence may be covalently linked. In some embodiments, the VH and the VL sequences are linked by a flexible linker sequence, e.g. a flexible linker sequence as described herein. The flexible linker sequence may be joined to ends of the VH sequence and VL sequence, thereby linking the VH and VL sequences. In some embodiments the VH and VL are joined via a linker sequence comprising, or consisting of, the amino acid sequence of SEQ ID NO:16 or 17.

In some embodiments, the CD30-binding domain comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:18.

In some embodiments the CD30-binding domain is capable of binding to CD30, e.g. in the extracellular domain of CD30. In some embodiments, the CD30-binding domain is capable of binding to the epitope of CD30 which is bound by antibody HRS3, e.g. within the region of amino acid positions 185-335 of human CD30 numbered according to SEQ ID NO:1, shown in SEQ ID NO:19 (Schlapschy et al., Protein Engineering, Design and Selection (2004) 17(12): 847-860, hereby incorporated by reference in its entirety).

In some embodiments, the target antigen is CD19. Accordingly, in some aspects and embodiments of the present disclosure the antigen-binding domain is a CD19-binding domain.

CD19 is the protein identified by UniProt P15391-1, v6. In this specification “CD19” refers to CD19 from any species and includes CD19 isoforms (e.g. P15391-2), fragments, variants (including mutants) or homologues from any species.

The CD19-binding domain may be derived from the antigen-binding moiety of an anti-CD19 antibody. Anti-CD19 antibodies include FMC63, described e.g. in Zola et al., Immunology and Cell Biology (1991) 69:411-422.

In some embodiments a CD19-binding domain according to the present disclosure comprises the CDRs of an anti-CD19 antibody. In some embodiments a CD19-binding domain according to the present disclosure comprises the VH and VL regions of an anti-CD19 antibody. In some embodiments a CD19-binding domain according to the present disclosure comprises a scFv comprising the VH and VL regions of an anti-CD19 antibody.

In some embodiments the antigen-binding domain of the present disclosure comprises:
a VH incorporating the following CDRs:
HC-CDR1 having the amino acid sequence of SEQ ID NO:37
HC-CDR2 having the amino acid sequence of SEQ ID NO:38
HC-CDR3 having the amino acid sequence of SEQ ID NO:39,
or a variant thereof in which one or two or three amino acids in one or more of HC-CDR1, HC-CDR2, or HC-CDR3 are substituted with another amino acid;
   and
a VL incorporating the following CDRs:
LC-CDR1 having the amino acid sequence of SEQ ID NO:40
LC-CDR2 having the amino acid sequence of SEQ ID NO:41
LC-CDR3 having the amino acid sequence of SEQ ID NO:42,
or a variant thereof in which one or two or three amino acids in one or more of LC-CDR1, LC-CDR2, or LC-CDR3 are substituted with another amino acid.

In some embodiments the antigen-binding domain comprises:
a VH comprising, or consisting of, an amino acid sequence having at least 80% sequence identity (e.g. at least 85%, 86%, 87%, 88%, 89%, 90%, 91%, 92%, 93%, 94%, 95%, 96%, 97%, 98%, 99% or 100%) to the amino acid sequence of SEQ ID NO:43:
   and
a VL comprising, or consisting of, an amino acid sequence having at least 80% sequence identity (e.g. at least 85%, 86%, 87%, 88%, 89%, 90%, 91%, 92%, 93%, 94%, 95%, 96%, 97%, 98%, 99% or 100%) to the amino acid sequence of SEQ ID NO:44.

In some embodiments, a CD19-binding domain may comprise or consist of a single chain variable fragment (scFv) comprising a VH sequence and a VL sequence as described herein. The VH sequence and VL sequence may be covalently linked. In some embodiments, the VH and the VL sequences are linked by a flexible linker sequence, e.g. a flexible linker sequence as described herein. The flexible linker sequence may be joined to ends of the VH sequence and VL sequence, thereby linking the VH and VL sequences. In some embodiments the VH and VL are joined via a linker sequence comprising, or consisting of, the amino acid sequence of SEQ ID NO:16 or 45.

In some embodiments, the CD19-binding domain comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:46.

In some embodiments the CD19-binding domain is capable of binding to CD19, e.g. in the extracellular domain of CD19. In some embodiments, the CD19-binding domain is capable of binding to the epitope of CD19 which is bound by antibody FMC63.

Transmembrane Domain

The CAR of the present disclosure comprises a transmembrane domain. A transmembrane domain refers to any three-dimensional structure formed by a sequence of amino acids which is thermodynamically stable in a biological membrane, e.g. a cell membrane. In connection with the present disclosure, the transmembrane domain may be an amino acid sequence which spans the cell membrane of a cell expressing the CAR.

The transmembrane domain may comprise or consist of a sequence of amino acids which forms a hydrophobic alpha helix or beta-barrel. The amino acid sequence of the transmembrane domain of the CAR of the present disclosure may be, or may be derived from, the amino acid sequence of a transmembrane domain of a protein comprising a transmembrane domain. Transmembrane domains are recorded in databases such as GenBank, UniProt, Swiss-Prot, TrEMBL, Protein Information Resource, Protein Data Bank, Ensembi, and InterPro, and/or can be identified/predicted e.g. using amino acid sequence analysis tools such as TMHMM (Krogh et al., 2001 J Mol Biol 305: 567-580).

In some embodiments, the amino acid sequence of the transmembrane domain of the CAR of the present disclosure may be, or may be derived from, the amino acid sequence of the transmembrane domain of a protein expressed at the cell surface. In some embodiments the protein expressed at the cell surface is a receptor or ligand, e.g. an immune receptor or ligand. In some embodiments the amino acid sequence of the transmembrane domain may be, or may be derived from, the amino acid sequence of the transmembrane domain of one of ICOS, ICOSL, CD86, CTLA-4, CD28, CD80, MHC class I α, MHC class II α, MHC class II β, CD3ε, CD3δ, CD3γ, CD3-ζ, TCRα TCRβ, CD4, CD8α, CD80, CD40, CD40L, PD-1, PD-L1, PD-L2, 4-1BB, 4-1BBL, OX40, OX40L, GITR, GITRL, TIM-3, Galectin 9, LAG3, CD27, CD70, LIGHT, HVEM, TIM-4, TIM-1, ICAM1, LFA-1, LFA-3, CD2, BTLA, CD160, LILRB4, LILRB2, VTCN1, CD2, CD48, 2B4, SLAM, CD30, CD30L, DR3, TL1A, CD226, CD155, CD112 and CD276. In some embodiments, the transmembrane is, or is derived from, the amino acid sequence of the transmembrane domain of CD28, CD3-ζ, CD8α, CD8β or CD4. In some embodiments, the transmembrane is, or is derived from, the amino acid sequence of the transmembrane domain of CD28.

In some embodiments, the transmembrane domain comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:20 or 48.

In some embodiments, the transmembrane domain comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:21.

In some embodiments, the transmembrane domain comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:22.

Signalling Domain

The chimeric antigen receptor of the present disclosure comprises a signalling domain. The signalling domain provides sequences for initiating intracellular signalling in cells expressing the CAR.

ITAM-Containing Sequence

The signalling domain comprises ITAM-containing sequence. An ITAM-containing sequence comprises one or more immunoreceptor tyrosine-based activation motifs (ITAMs). ITAMs comprise the amino acid sequence YXXL/I (SEQ ID NO:23), wherein “X” denotes any amino acid. In ITAM-containing proteins, sequences according to SEQ ID NO:23 are often separated by 6 to 8 amino acids; YXXL/I(X)₆₋₈YXXL/I (SEQ ID NO:24). When phosphate groups are added to the tyrosine residue of an ITAM by tyrosine kinases, a signalling cascade is initiated within the cell.

In some embodiments, the signalling domain comprises one or more copies of an amino acid sequence according to SEQ ID NO:23 or SEQ ID NO:24. In some embodiments, the signalling domain comprises at least 1, 2, 3, 4, 5 or 6 copies of an amino acid sequence according to SEQ ID NO:23. In some embodiments, the signalling domain comprises at least 1, 2, or 3 copies of an amino acid sequence according to SEQ ID NO:24.

In some embodiments, the signalling domain comprises an amino acid sequence which is, or which is derived from, the amino acid sequence of an ITAM-containing sequence of a protein having an ITAM-containing amino acid sequence. In some embodiments the signalling domain comprises an amino acid sequence which is, or which is derived from, the amino acid sequence of the intracellular domain of one of CD3-ζ, FcγRI, CD3ε, CD3δ, CD3γ, CD79α, CD79β, FcγRIIA, FcγRIIC, FcγRIIA, FcγRIV or DAP12. In some embodiments the signalling domain comprises an amino acid sequence which is, or which is derived from, the intracellular domain of CD3-ζ.

In some embodiments, the signalling domain comprises an amino acid sequence which comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:25.

Costimulatory Sequence

The signalling domain may additionally comprise one or more costimulatory sequences. A costimulatory sequence is an amino acid sequence which provides for costimulation of the cell expressing the CAR of the present disclosure. Costimulation promotes proliferation and survival of a CAR-expressing cell upon binding to the target antigen, and may also promote cytokine production, differentiation, cytotoxic function and memory formation by the CAR-expressing cell. Molecular mechanisms of T cell costimulation are reviewed in Chen and Flies, (2013) Nat Rev Immunol 13(4):227-242.

A costimulatory sequence may be, or may be derived from, the amino acid sequence of a costimulatory protein. In some embodiments the costimulatory sequence is an amino acid sequence which is, or which is derived from, the amino acid sequence of the intracellular domain of a costimulatory protein.

Upon binding of the CAR to the target antigen, the costimulatory sequence provides costimulation to the cell expressing the CAR of the kind which would be provided by the costimulatory protein from which the costimulatory sequence is derived upon ligation by its cognate ligand. By way of example in the case of a CAR comprising a signalling domain comprising a costimulatory sequence derived from CD28, binding to the target antigen triggers signalling in the cell expressing the CAR of the kind that would be triggered by binding of CD80 and/or CD86 to CD28. Thus, a costimulatory sequence is capable of delivering the costimulation signal of the costimulatory protein from which the costimulatory sequence is derived.

In some embodiments, the costimulatory protein may be a member of the B7-CD28 superfamily (e.g. CD28, ICOS), or a member of the TNF receptor superfamily (e.g. 4-1BB, OX40, CD27, DR3, GITR, CD30, HVEM). In some embodiments, the costimulatory sequence is, or is derived from, the intracellular domain of one of CD28, 4-1BB, ICOS, CD27, OX40, HVEM, CD2, SLAM, TIM-1, CD30, GITR, DR3, CD226 and LIGHT. In some embodiments, the costimulatory sequence is, or is derived from, the intracellular domain of CD28.

In some embodiments the signalling domain comprises more than one non-overlapping costimulatory sequences. In some embodiments the signalling domain comprises 1, 2, 3, 4, 5 or 6 costimulatory sequences. Plural costimulatory sequences may be provided in tandem.

Whether a given amino acid sequence is capable of initiating signalling mediated by a given costimulatory protein can be investigated e.g. by analysing a correlate of signalling mediated by the costimulatory protein (e.g. expression/activity of a factor whose expression/activity is upregulated or downregulated as a consequence of signalling mediated by the costimulatory protein).

Costimulatory proteins upregulate expression of genes promoting cell growth, effector function and survival through several transduction pathways. For example, CD28 and ICOS signal through phosphatidylinositol 3 kinase (PI3K) and AKT to upregulate expression of genes promoting cell growth, effector function and survival through NF-κB, mTOR, NFAT and AP1/2. CD28 also activates AP1/2 via CDC42/RAC1 and ERK1/2 via RAS, and ICOS activates C-MAF. 4-1BB, OX40, and CD27 recruit TNF receptor associated factor (TRAF) and signal through MAPK pathways, as well as through PI3K.

In some embodiments the signalling domain comprises a costimulatory sequence which is, or which is derived from CD28.

In some embodiments, the signalling domain comprises a costimulatory sequence which comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:26.

Kofler et al. Mol. Ther. (2011) 19: 760-767 describes a variant CD28 intracellular domain in which the Ick kinase binding site is mutated in order to reduce induction of IL-2 production on CAR ligation, in order to minimise regulatory T cell-mediated suppression of CAR-T cell activity. The amino acid sequence of the variant CD28 intracellular domain is shown in SEQ ID NO:27.

In some embodiments, the signalling domain comprises a costimulatory sequence which comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:27.

In some embodiments, the signalling domain comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:28.

In some embodiments the signalling domain comprises a costimulatory sequence which is, or which is derived from 4-1BB.

In some embodiments, the signalling domain comprises a costimulatory sequence which comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:49.

In some embodiments, the signalling domain comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:50.

Hinge Region

The CAR may further comprise a hinge region. The hinge region may be provided between the antigen-binding domain and the transmembrane domain. The hinge region may also be referred to as a spacer region. A hinge region is an amino acid sequence which provides for flexible linkage of the antigen-binding and transmembrane domains of the CAR.

The presence, absence and length of hinge regions has been shown to influence CAR function (reviewed e.g. in Dotti et al., Immunol Rev (2014) 257(1) supra).

In some embodiments, the CAR comprises a hinge region which comprises, or consists of, an amino acid sequence which is, or which is derived from, the CH1-CH2 hinge region of human IgG1, a hinge region derived from CD8α, e.g. as described in WO 2012/031744 A1, or a hinge region derived from CD28, e.g. as described in WO 2011/041093 A1. In some embodiments, the CAR comprises a hinge region derived from the CH1-CH2 hinge region of human IgG1.

In some embodiments, the hinge region comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:29 or 30.

In some embodiments, the CAR comprises a hinge region derived from the CH1-CH2 hinge region of human IgG4.

In some embodiments, the hinge region comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:47.

In some embodiments, the CAR comprises a hinge region which comprises, or consists of, an amino acid sequence which is, or which is derived from, the CH2-CH3 region (i.e. the Fc region) of human IgG1.

In some embodiments, the hinge region comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:31.

Hombach et al., Gene Therapy (2010) 17:1206-1213 describes a variant CH2-CH3 region for reduced activation of FcγR-expressing cells such as monocytes and NK cells. The amino acid sequence of the variant CH2-CH3 region is shown in SEQ ID NO:32.

In some embodiments, the hinge region comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:32.

In some embodiments, the hinge region comprises, or consists of: an amino acid sequence which is, or which is derived from, the CH1-CH2 hinge region of human IgG1, and an amino acid sequence which is, or which is derived from, the CH2-CH3 region (i.e. the Fc region) of human IgG1.

In some embodiments, the hinge region comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:33.

Additional Sequences

Signal Peptide

The CAR may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides. Signal peptides are known for many proteins, and are recorded in databases such as GenBank, UniProt and Ensembl, and/or can be identified/predicted e.g. using amino acid sequence analysis tools such as SignalP (Petersen et al., 2011 Nature Methods 8: 785-786) or Signal-BLAST (Frank and Sippi, 2008 Bioinformatics 24: 2172-2176).

The signal peptide may be present at the N-terminus of the CAR, and may be present in the newly synthesised CAR. The signal peptide provides for efficient trafficking of the CAR to the cell surface. Signal peptides are removed by cleavage, and thus are not comprised in the mature CAR expressed by the cell surface.

In some embodiments, the signal peptide comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:34. In some embodiments, the signal peptide comprises, or consists of, an amino acid sequence having at least 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:51.

Linker Sequences and Further Functional Sequences

In some embodiments the CAR comprises one or more linker sequences between the different domains (i.e. the antigen-binding domain, hinge region, transmembrane domain, signalling domain). In some embodiments the CAR comprises one or more linker sequences between subsequences of the domains (e.g. between VH and VL of an antigen-binding domain).

Linker sequences are known to the skilled person, and are described, for example in Chen et al., Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues. In some embodiments, the linker sequence comprises at least one glycine residue and/or at least one serine residue. In some embodiments the linker sequence consists of glycine and serine residues. In some embodiments, the linker sequence has a length of 1-2, 1-3, 1-4, 1-5, 1-10, 1-20, 1-30, 1-40 or 1-50 amino acids.

In some embodiments a linker sequence comprises, or consists, of the amino acid sequence shown in SEQ ID NO:16 or 45. In some embodiments a linker sequence comprises, or consists, of 1, 2, 3, 4 or 5 tandem copies of the amino acid sequence shown in SEQ ID NO:16 or 45.

The CARs may additionally comprise further amino acids or sequences of amino acids. For example, the antigen-binding molecules and polypeptides may comprise amino acid sequence(s) to facilitate expression, folding, trafficking, processing, purification or detection. For example, the CAR may comprise a sequence encoding a His, (e.g. 6×His), Myc, GST, MBP, FLAG, HA, E, or Biotin tag, optionally at the N or C-terminus. In some embodiments the CAR comprises a detectable moiety, e.g. a fluorescent, luminescent, immuno-detectable, radio, chemical, nucleic acid or enzymatic label.

Particular Exemplary CARs

In some embodiments of the present disclosure, the CAR comprises, or consists of:
An antigen-binding domain comprising or consisting of an amino acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:18;
A hinge region comprising or consisting of an amino acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:33;
A transmembrane domain comprising or consisting of an amino acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:20; and
A signalling domain comprising or consisting of an amino acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:28.

In some embodiments of the present disclosure, the CAR comprises, or consists of an amino acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:35 or 36.

In some embodiments, the CAR is selected from an embodiment of a CD30-specific CAR described in Hombach et al. Cancer Res. (1998) 58(6):1116-9, Hombach et al. Gene Therapy (2000) 7:1067-1075, Hombach et al. J Immunother. (1999) 22(6):473-80, Hombach et al. Cancer Res. (2001) 61:1976-1982, Hombach et al. J Immunol (2001) 167:6123-6131, Savoldo et a. Blood (2007) 110(7):2620-30, Koehler et al. Cancer Res. (2007) 67(5):2265-2273, Di Stasi et al. Blood (2009) 113(25):6392-402, Hombach et al. Gene Therapy (2010) 17:1206-1213, Chmielewski et a. Gene Therapy (2011) 18:62-72, Kofier et al. Mol. Ther. (2011) 19(4):760-767, Gilham, Abken and Pule. Trends in Mol. Med. (2012) 18(7):377-384, Chmielewski et al. Gene Therapy (2013) 20:177-186, Hombach et al. Mol. Ther. (2016) 24(8):1423-1434, Ramos et a. J. Clin. Invest. (2017) 127(9):3462-3471, WO 2015/028444 A1 or WO 2016/008973 A1, all of which are hereby incorporated by reference in their entirety.

In some embodiments of the present disclosure, the CAR comprises, or consists of:
An antigen-binding domain comprising or consisting of an amino acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:46;
A hinge region comprising or consisting of an amino acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:47;
A transmembrane domain comprising or consisting of an amino acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:48; and
A signalling domain comprising or consisting of an amino acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:50.

In some embodiments of the present disclosure, the CAR comprises, or consists of an amino acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:52 or 53.

CAR-Expressing, Virus-Specific Immune Cells

The present disclosure relates to virus-specific immune cells comprising/expressing chimeric antigen receptors (CARs).

CAR-expressing virus-specific immune cells may express or comprise a CAR according to the present disclosure. CAR-expressing virus-specific immune cells may comprise or express nucleic acid encoding a CAR according to the present disclosure. It will be appreciated that a CAR-expressing cell comprises the CAR it expresses. It will also be appreciated that a cell expressing nucleic acid encoding a CAR also expresses and comprises the CAR encoded by the nucleic acid.

A virus-specific immune cell comprising a CAR/nucleic acid encoding a CAR according to the present disclosure may be characterised by reference to functional properties of the cells.

In some embodiments a virus-specific immune cell comprising a CAR/nucleic acid encoding a CAR according to the present disclosure displays one or more of the following properties:
(a) expression of one or more cytotoxic/effector factors (e.g. IFNγ, granzyme, perforin, granulysin, CD107a, TNFα, FASL), proliferation/population expansion, and/or growth factor (e.g. IL-2) expression in response to cells expressing the target antigen for which the CAR is specific, in response to cells infected with the virus for which the virus-specific immune cell is specific, and/or in response to cells presenting a peptide of an antigen of the virus for which the virus-specific immune cell is specific;
(b) cytotoxicity to cells expressing the target antigen for which the CAR is specific, cells infected with the virus for which the virus-specific immune cell is specific, and/or cells presenting a peptide of an antigen of the virus for which the virus-specific immune cell is specific;
(c) no cytotoxicity (i.e. above baseline) to cells which do not express the target antigen for which the CAR is specific, cells which are not infected with the virus for which the virus-specific immune cell is specific, and/or cells which do not present a peptide of an antigen of the virus for which the virus-specific immune cell is specific;
(d) anti-cancer activity (e.g. cytotoxicity to cancer cells, tumor growth inhibition, reduction of metastasis, etc.) against cancer comprising cells expressing the target antigen for which the CAR is specific, cancer comprising cells infected with the virus for which the virus-specific immune cell is specific, and/or cancer comprising cells presenting a peptide of an antigen of the virus for which the virus-specific immune cell is specific; and
(e) cytotoxicity to alloreactive immune cells, e.g. alloreactive immune cells expressing the target antigen for which the CAR is specific.

Cell proliferation/population expansion can be investigated by analysing cell division or the number of cells over a period of time. Cell division can be analysed, for example, by in vitro analysis of incorporation of ³H-thymidine or by CFSE dilution assay, e.g. as described in Fulcher and Wong, Immunol Cell Biol (1999) 77(6): 559-564, hereby incorporated by reference in its entirety. Proliferating cells can also be identified by analysis of incorporation of 5-ethynyl-2′-deoxyuridine (EdU) by an appropriate assay, as described e.g. in Buck et al., Biotechniques. 2008 June; 44(7):927-9, and Sali and Mitchison, PNAS USA 2008 Feb. 19; 105(7): 2415-2420, both hereby incorporated by reference in their entirety.

As used herein, “expression” may be gene or protein expression. Gene expression encompasses transcription of DNA to RNA, and can be measured by various means known to those skilled in the art, for example by measuring levels of mRNA by quantitative real-time PCR (qRT-PCR), or by reporter-based methods. Similarly, protein expression can be measured by various methods well known in the art, e.g. by antibody-based methods, for example by western blot, immunohistochemistry, immunocytochemistry, flow cytometry, ELISA, ELISPOT, or reporter-based methods.

Cytotoxicity and cell killing can be investigated, for example, using any of the methods reviewed in Zaritskaya et al., Expert Rev Vaccines (2011), 9(6):601-616, hereby incorporated by reference in its entirety. Examples of in viro assays of cytotoxicity/cell killing assays include release assays such as the ⁵¹Cr release assay, the lactate dehydrogenase (LDH) release assay, the 3-(4,5-dimethylthiazol-2-yl)-2,5-diphenyl tetrazolium bromide (MTT) release assay, and the calcein-acetoxymethyl (calcein-AM) release assay. These assays measure cell killing based on the detection of factors released from lysed cells. Cell killing by a given cell type can be analysed e.g. by co-culturing the test cells with the given cell type, and measuring the number/proportion of cells viable/dead test cells after a suitable period of time.

Cells may be evaluated for anti-cancer activity by analysis in an appropriate in vitro assays or in vivo models of the cancer.

In some embodiments, CD30-specific CAR-expressing, EBV-specific immune cells of the present disclosure display one or more of the following properties:
(a) expression of one or more cytotoxic/effector factors (e.g. IFNγ, granzyme, perforin, granulysin, CD107a, TNFα, FASL) in response to cells expressing CD30, in response to cells infected with EBV, and/or in response to cells presenting a peptide of an EBV antigen;
(b) cytotoxicity to cells expressing CD30, cells infected with EBV, and/or cells presenting a peptide of an EBV antigen;
(c) no cytotoxicity (i.e. above baseline) to cells which do not express CD30, cells which are not infected with EBV, and/or cells which do not present a peptide of an EBV antigen;
(d) anti-cancer activity (e.g. cytotoxicity to cancer cells, tumor growth inhibition, reduction of metastasis, etc.) against cancer comprising cells expressing CD30, cancer comprising cells infected with EBV, and/or cancer comprising cells presenting a peptide of an EBV antigen; and
(e) cytotoxicity to alloreactive immune cells, e.g. alloreactive immune cells expressing CD30.

In some embodiments in accordance with the various aspects of the present disclosure, virus-specific immune cells may comprise/express more than one (e.g. 2, 3, 4, etc.) CAR.

In some embodiments, virus-specific immune cells may comprise/express more than one, non-identical CAR. Virus-specific immune cells comprising/expressing more than one non-identical CAR may comprise/express CARs specific for non-identical target antigens. For example, Example 4 herein describes virus-specific immune cells comprising/expressing a CD30-specific CAR and a CD19-specific CAR. Each of the non-identical target antigens may independently be a target antigen as described herein. In some embodiments each non-identical target antigen is independently a cancer cell antigen as described herein.

In some embodiments, one of the non-identical target antigens is CD30. In some embodiments, a virus-specific immune cell comprising/expressing more than one, non-identical CAR comprises: a CD30-specific CAR, and a CAR specific for a target antigen other than CD30.

Producing CAR-Expressing, Virus-Specific Immune Cells

Methods for generating/expanding populations of virus-specific immune cells in vitro/ex vivo are well known to the skilled person. Typical culture conditions (i.e. cell culture media, additives, temperature, gaseous atmosphere), cell numbers, culture periods, etc. can be determined by reference e.g. to Ngo et al., J Immunother. (2014) 37(4):193-203, which is hereby incorporated by reference in its entirety.

Conveniently, cultures of cells according to the present disclosure may be maintained at 37° C. in a humidified atmosphere containing 5% CO₂. The cells of cell cultures can be established and/or maintained at any suitable density, as can readily be determined by the skilled person. For example, cultures may be established at an initial density of ˜0.5×10⁸ to ˜5×10⁶ cells/ml of the culture (e.g. −1×10⁶ cells/m).

Cultures can be performed in any vessel suitable for the volume of the culture, e.g. in wells of a cell culture plate, cell culture flasks, a bioreactor, etc. In some embodiments cells are cultured in a bioreactor, e.g. a bioreactor described in Somerville and Dudley, Oncoimmunology (2012) 1(8):1435-1437, which is hereby incorporated by reference in its entirety. In some embodiments cells are cultured in a GRex cell culture vessel, e.g. a GRex flask or a GRex 100 bioreactor.

The methods generally comprise culturing populations of immune cells (e.g. heterogeneous populations of immune cells, e.g. peripheral blood mononuclear cells; PBMCs) comprising cells having antigen-specific receptors in the presence of antigen-presenting cells (APCs) presenting viral antigen peptide:MHC complexes, under conditions providing appropriate costimulation and signal amplification so as to cause activation and expansion. The APCs may be infected with virus encoding, or may comprise/express, the viral antigen/peptide(s), and present the viral antigen peptide in the context of an MHC molecule. Stimulation causes T cell activation, and promotes cell division (proliferation), resulting in generation and/or expansion of a population of T cells specific for the viral antigen. The process of T cell activation is well known to the skilled person and described in detail, for example, in Immunobiology, 5th Edn. Janeway C A Jr, Travers P, Walport M, et al. New York: Gariand Science (2001), Chapter 8, which is incorporated by reference in its entirety.

The population of cells obtained following stimulation is enriched for T cells specific for the virus as compared to the population prior to stimulation (i.e. the virus-specific T cells are present at an increased frequency in the population following stimulation). In this way, a population of T cells specific for the virus is expanded/generated out of a heterogeneous population of T cells having different specificities. A population of T cells specific for a virus may be generated from a single T cell by stimulation and consequent cell division. An existing population of T cells specific for a virus may be expanded by stimulation and consequent cell division of cells of the population of virus-specific T cells.

Aspects and embodiments of the present disclosure relate particularly to EBV-specific immune cells. Accordingly, in some embodiments, the virus may be EBV, and the viral antigen(s) may be EBV antigen(s). Methods for generating/expanding populations of EBV-specific immune cells are described e.g. in WO 2013/088114 A1, Lapteva and Vera, Stem Cells Int. (2011): 434392, Straathof et al., Blood (2005) 105(5): 1898-1904, WO 2017/202478 A1, WO 2018/052947 A1 and WO 2020/214479 A1, all of which are hereby incorporated by reference in their entirety.

The methods involve steps in which T cells comprising T cell receptors (TCRs) specific for EBV antigen peptide:MHC complex are stimulated by APCs presenting the EBV antigen peptide:MHC complex for which the TCR is specific. The APCs are infected with virus encoding, or comprise/express the EBV antigen/peptide(s), and present the EBV antigen peptide in the context of an MHC molecule. Stimulation causes T cell activation, and promotes cell division (proliferation), resulting in generation and/or expansion of a population of T cells specific for the EBV antigen.

The methods typically comprise stimulating immune cells specific for a virus/viral antigen by contacting populations of immune cells with peptide(s) corresponding to EBV antigen(s) or APCs presenting peptide(s) corresponding to viral antigen(s). Such method steps may be referred to herein as “stimulations” or“stimulation steps”. Such method steps typically involve maintenance of the cells in culture in vitro/ex vivo, and may be referred to as “stimulation cultures”.

In some embodiments, the methods comprise one or more additional stimulation steps. That is, in some embodiments the methods comprise one or more further steps of re-stimulating the cells obtained by a stimulation step. Such further stimulation steps may be referred to herein as “re-stimulations” or “re-stimulation steps”. Such method steps typically involve maintenance of the cells in culture in vitro/ex vivo, and may be referred to as “re-stimulation cultures”.

It will be appreciated that “contacting” PBMCs (for stimulations) or populations of cells obtained by a stimulation step described herein (for re-stimulations) with peptide(s) corresponding to viral antigen(s) generally involves culturing the PBMCs/population of cells in vitro/ex vivo in cell culture medium comprising the peptide(s). Similarity, it will be appreciated that “contacting” PBMCs/populations of cells with APCs presenting peptide(s) corresponding to viral antigen(s) generally involves co-culturing the APCs and the PBMCs/population of cells in vitro/ex vivo in cell culture medium.

In some embodiments, the methods comprise contacting PBMCs with peptide(s) corresponding to viral antigen(s) (e.g. EBV antigen(s)). In such embodiments, APCs within the population of PBMCs (e.g. dendritic cells, macrophages and B cells) internalise (e.g. by phagocytosis, pinocytosis), process and present the antigens on MHC class I molecules (cross-presentation) and/or MHC class II molecules, for subsequent activation of CD8+ and/or CD4+ T cells within the population of PBMCs.

A peptide which “corresponds to” a reference antigen comprises or consists of an amino acid sequence of the reference antigen. For example, a peptide “corresponding to” EBNA1 of EBV comprises or consists of a sequence of amino acids which is found within the amino acid sequence of EBNA1 (i.e. is a subsequence of the amino acid sequence of EBNA1). Peptides employed herein typically have a length of 5-30 amino acids, e.g. one of 5-25 amino acids, 10-20 amino acids, or 12-18 amino acids. In some embodiments, peptides have a length of one of 5, 6, 7, 8, 9, 10, 11, 12, 13, 14, 15, 16, 17, 18, 19 or 20 amino acids. In some embodiments, peptides have a length of about 15 amino acids. “Peptides” as used herein may refer to populations comprising non-identical peptides.

In some embodiments, the methods employ peptides corresponding to more than one antigen. In such embodiments, there is at least one peptide which corresponds to each of the antigens. For example, where the methods employ peptides corresponding to EBNA1 and LMP1, the peptides comprise at least one peptide corresponding to EBNA1, and at least one peptide corresponding to LMP1.

In some embodiments the methods employ peptides corresponding to all or part of a reference antigen. Peptides corresponding to all of a given antigen cover the full length of the amino acid sequence of the antigen. That is to say that together, the peptides contain all of the amino acids of the amino acid sequence of the given antigen. Peptides corresponding to part of a given antigen cover part of the amino acid sequence of the antigen. In some embodiments where peptides cover part of the amino acid sequence of the antigen, the peptides together may cover e.g. greater than 10%, e.g. greater than one of 15%, 20%, 25%, 30%, 35%, 40%, 45%, 50%, 55%, 60%, 65%, 70%, 75%, 80%, 85%, 90% or 95% of the amino acid sequence of the antigen.

In some embodiments the methods employ overlapping peptides. “Overlapping” peptides have amino acids, and more typically sequences of amino acids, in common. By way of illustration, a first peptide consists of an amino acid sequence corresponding to positions 1 to 15 of the amino acid sequence of EBNA1, and a second peptide consists of an amino acid sequence corresponding to positions 5 to 20 of the amino acid sequence of EBNA1. The first and second peptides are overlapping peptides corresponding to EBNA1, overlapping by 11 amino acids. In some embodiments overlapping peptides overlap by one of 1-20, 5-20, 8-15 or 10-12 amino acids. In some embodiments overlapping peptides overlap by one of 1, 2, 3, 4, 5, 6, 7, 8, 9, 10, 11, 12, 13, 14 or 15 amino acids. In some embodiments overlapping peptides overlap by 11 amino acids.

In some embodiments, the methods employ peptides having a length of 5-30 amino acids, overlapping by 1-20 amino acids, corresponding to all or part of a given reference antigen.

In some embodiments, the methods employ peptides having a length of 15 amino acids, overlapping by 11 amino acids, corresponding to all of a given reference antigen. Mixtures of such peptides may be referred to herein as “pepmix peptide pools” or “pepmixes” for a given antigen. For example, “EBNA1 pepmix” used in Example 1 herein refers to a pool of 158, 15mer peptides overlapping by 11 amino acids, spanning the full length of the amino acid sequence for EBNA1 as shown in UniProt: P03211-1, v1.

In some embodiments in accordance with various aspects of the present disclosure, “peptides corresponding to” a given viral antigen may be a pepmix for the antigen.

In particular embodiments, the methods employ peptides corresponding to one or more EBV antigens. In particular embodiments, the methods employ pepmixes for one or more EBV antigens. In some embodiments, the one or more EBV antigens are selected from: an EBV latent antigen, e.g. a type III latency antigen (e.g. EBNA1, EBNA-LP, LMP1, LMP2A, LMP2B, BARF1, EBNA2, EBNA3A, EBNA3B or EBNA3C), a type II latency antigen (e.g. EBNA1, EBNA-LP, LMP1, LMP2A, LMP2B or BARF1), or a type I latency antigen, (e.g. EBNA1 or BARF1), an EBV lytic antigen, e.g. an immediate-early lytic antigen (e.g. BZLF1, BRLF1 or BMRF1), an early lytic antigen (e.g. BMLF1, BMRF1, BXLF1, BALF1, BALF2, BARF1, BGLF5, BHRF1, BNLF2A, BNLF2B, BHLF1, BLLF2, BKRF4, BMRF2, FU or EBNA1-FUK), and a late lytic antigen (e.g. BALF4, BILF1, BILF2, BNFR1, BVRF2, BALF3, BALF5, BDLF3 orgp350).

In some embodiments in accordance with various aspects of the present disclosure, the one or more EBV antigens are or comprise EBV lytic antigens selected from BZLF1, BRLF1, BMLF1, BMRF1, BXLF1, BALF1, BALF2, BGLF5, BHRF1, BNLF2A, BNLF2B, BHLF1, BLLF2, BKRF4, BMRF2, BALF4, BILF1, BILF2, BNFR1, BVRF2, BALF3, BALF5 and BDLF3. In some embodiments the one or more EBV antigens are or comprise EBV lytic antigens selected from BZLF1, BRLF1, BMLF1, BMRF1, BALF2, BNLF2A, BNLF2B, BMRF2 and BDLF3.

In some embodiments the one or more EBV antigens are or comprise EBV latent antigens selected from EBNA1, EBNA-LP, EBNA2, EBNA3A, EBNA3B, EBNA3C, BARF1, LMP1, LMP2A and LMP2B. In some embodiments the one or more EBV antigens are or comprise EBV latent antigens selected from EBNA1, LMP1, LMP2A and LMP2B.

In some embodiments, the one or more EBV antigens are selected from: EBNA1, LMP1, LMP2, BARF1, BZLF1, BRLF1, BMLF1, BMRF1, BMRF2, BALF2, BNLF2A and BNLF2B.

In some embodiments, the methods employ peptides corresponding to EBNA1, LMP1, LMP2, BARF1, BZLF1, BRLF1, BMLF1, BMRF1, BMRF2, BALF2, BNLF2A and BNLF2B. In some embodiments, the methods employ pepmixes for EBNA1, LMP1, LMP2, BARF1, BZLF1, BRLF1, BMLF1, BMRF1, BMRF2, BALF2, BNLF2A and BNLF2B.

In some embodiments, the methods comprise contacting PBMCs (e.g. PBMCs depleted of CD45RA-positive cells) with peptide(s) corresponding to EBNA1, LMP1, LMP2, BARF1, BZLF1, BRLF1, BMLF1, BMRF1, BMRF2, BALF2, BNLF2A and BNLF2B. In some embodiments, the methods comprise contacting PBMCs (e.g. PBMCs depleted of CD45RA-positive cells) with pepmixes for EBNA1, LMP1, LMP2, BARF1, BZLF1, BRLF1, BMLF1, BMRF1, BMRF2, BALF2, BNLF2A and BNLF2B.

In some embodiments, the methods comprise contacting the population of cells obtained by a stimulation step described herein with peptide(s) corresponding to viral antigen(s). In such embodiments, APCs within the population of cells (e.g. dendritic cells, macrophages and B cells) internalise (e.g. by phagocytosis, pinocytosis), process and present the antigens on MHC class I molecules (cross-presentation) and/or MHC class II molecules, for subsequent re-stimulation of CD8+ and/or CD4+ T cells within the population of cells.

In some embodiments, the methods comprise contacting PBMCs with APCs presenting peptide(s) corresponding to viral antigen(s). In some embodiments, the methods comprise contacting the population of cells obtained by a stimulation step described herein with APCs presenting peptide(s) corresponding to viral antigen(s).

In some embodiments, the methods comprise contacting PBMCs with EBV-LCLs. Production of EBV-specific immune cells by stimulating PBMCs with EBV-LCLs is described e.g. in Straathof et al., Blood (2005) 105(5): 1898-1904, which is incorporated by reference hereinabove.

EBV-LCLs may be prepared by infection of PBMCs with EBV, and collecting the immortalized EBV infected cells after long-term culture, e.g. as described in Hui-Yuen et al., J Vis Exp (2011) 57: 3321, and Hussain and Mulherkar, Int J Mol Cell Med (2012) 1(2): 75-87 (both hereby incorporated by reference in their entirety). EBV-specific T cells may be prepared by co-culture of PBMCs isolated from blood samples from healthy donors with autologous, gamma-irradiated EBV-LCLs.

Co-culture of T cells and APCs in stimulations and re-stimulations is performed in cell culture medium. The cell culture medium can be any cell culture medium in which T cells and APCs according to the present disclosure can be maintained in culture in vitro/ex vivo. Culture medium suitable for use in the culture of lymphocytes is well known to the skilled person, and includes, for example, RPMI-1640 medium, AIM-V medium, Iscoves medium, etc.

In some embodiments, cell culture medium may comprise RPMI-1640 medium (e.g. Advanced RPMI-1640 medium) and/or Click's medium (also known as Eagle's Ham's amino acids (EHAA) medium). The compositions of these media are well known to the skilled person. The formulation of RPMI-1640 medium is described in e.g. Moore et al., JAMA (1967) 199:519-524, and the formulation of Click's medium is described in Click et al., Cell Immunol (1972) 3:264-276. RPMI-1640 medium can be obtained from e.g. ThermoFisher Scientific, and Click's medium can be obtained from e.g. Sigma-Aldrich (Catalog No. C5572). Advanced RPMI-1640 medium can be obtained from e.g. ThermoFisher Scientific (Catalog No. 12633012).

In some embodiments, the methods involve culturing PBMCs that have been contacted with peptide(s) corresponding to viral antigen(s) (e.g. EBV antigen(s)), or in the presence of APCs presenting peptide(s) corresponding to viral antigen(s), in cell culture medium comprising RPMI-1640 medium and Click's medium. In some embodiments, the methods involve culturing the population of cells obtained by a stimulation step described herein that have been contacted with peptide(s) corresponding to viral antigen(s), or in the presence of APCs presenting peptide(s) corresponding to viral antigen(s), in cell culture medium comprising RPMI-1640 medium and Click's medium.

In some embodiments the cell culture medium comprises (by volume) 25-65% RPMI-1640 medium, and 25-65% Click's medium. In some embodiments the cell culture medium comprises 30-60% RPMI-1640 medium, and 30-60% Click's medium. In some embodiments the cell culture medium comprises 35-55% RPMI-1640 medium, and 35-55% Click's medium. In some embodiments the cell culture medium comprises 40-50% RPMI-1640 medium, and 40-50% Click's medium. In some embodiments the cell culture medium comprises 45% RPMI-1640 medium, and 45% Click's medium. In particular embodiments, the cell culture medium comprises 47.5% RPMI-1640 medium, and 47.5% Click's medium.

In some embodiments, the cell culture medium may comprise one or more cell culture medium additives. Cell culture medium additives are well known to the skilled person, and include antibiotics (e.g. penicillin, streptomycin), growth factor-rich additives such as serum (e.g. human serum, fetal bovine serum (FBS), bovine serum albumin (BSA)), L-glutamine, cytokines/growth factors, etc.

In some embodiments, the cell culture medium comprises (by volume) 2.5-20% (e.g. 5%) growth factor-rich additive, e.g. 5-20% FBS, e.g. 7.5-15% FBS, or 10% FBS. In some embodiments, the cell culture medium comprises 0.5-5% GlutaMax, e.g. 1% GlutaMax. In some embodiments, the cell culture medium comprises 0.5-5% Pen/Strep, e.g. 1% Pen/Strep.

In particular embodiments, the cell culture medium comprises human platelet lysate. In some embodiments, the cell culture medium comprises (by volume) 1-20% (e.g. 5%) human platelet lysate, e.g. 2.5-20% human platelet lysate, e.g. 2.5-15%, 2.5-10%, or 5% human platelet lysate. Human platelet lysate can be obtained from e.g. Sexton Biotechnologies.

In particular embodiments, the cell culture medium comprises L-glutamine. In particular embodiments, the cell culture medium comprises 0.5-10 mM L-glutamine, e.g. 1-5 mM L-glutamine, e.g. 2 mM L-glutamine.

APCs according to the present disclosure may be professional APCs. Professional APCs are specialised for presenting antigens to T cells; they are efficient at processing and presenting MHC-peptide complexes at the cell surface, and express high levels of costimulatory molecules. Professional APCs include dendritic cells (DCs), macrophages, and B cells. Non-professional APCs are other cells capable of presenting MHC-peptide complexes to T cells, in particular MHC Class I-peptide complexes to CD8+ T cells.

In some embodiments the APC is an APC capable of cross-presentation on MHC class I of antigen internalised by the APC (e.g. taken-up by endocytosis/phagocytosis). Cross-presentation on MHC class I of internalized antigens to CD8+ T cells is described e.g. in Alloatti et al., Immunological Reviews (2016), 272(1): 97-108, which is hereby incorporated by reference in its entirety. APCs capable of cross-presentation include e.g. dendritic cells (DCs), macrophages, B cells and sinusoidal endothelial cells.

As explained herein, in some embodiments APCs for stimulating immune cells specific for viral antigen(s) are comprised within the population of cells (e.g. PBMCs) comprising the immune cells specific for viral antigen(s), from which populations of cells specific for viral antigen(s) are to be expanded. In such embodiments, APCs may be e.g. dendritic cells, macrophages, B cells or any other cell type within the population of cells which is capable of presenting antigen(s) to immune cells specific for viral antigen(s).

In some embodiments the methods employ APCs that have been modified to express/comprise viral antigen(s)/peptide(s) thereof. In some embodiments, the APCs may present peptide(s) corresponding to viral antigen(s) as a result of having been contacted with the peptide(s), and having internalised them. In some embodiments, APCs may have been “pulsed” with the peptide(s), which generally involves culturing APCs in vitro in the presence of the peptide(s), for a period of time sufficient for the APCs to internalise the peptide(s).

In some embodiments the APCs may present peptide(s) corresponding to viral antigen(s) as a result of expression of nucleic acid encoding the antigen within the cell. APCs may comprise nucleic acid encoding viral antigen(s) as a consequence of their having been infected with the virus (e.g. in the case of EBV-infected B cells, e.g. LCLs). APCs may comprise nucleic acid encoding viral antigen(s) as a consequence of nucleic acid encoding the antigen(s) having been introduced into the cell, e.g. via transfection, transduction, electroporation, etc. Nucleic acid encoding viral antigen(s) may be provided in a plasmid/vector.

In some embodiments, APCs are selected from activated T cells (ATCs), dendritic cells, B cells (including e.g. LCLs), and artificial antigen presenting cells (aAPCs) such as those described in Neal et al., J Immunol Res Ther (2017) 2(1):68-79 and Turtle and Riddell Cancer J. (2010) 16(4):374-381.

In some embodiments APCs are autologous with respect to the population of cells with which they are to be co-cultured for the generation/expansion of populations of immune cells comprising immune cells specific for viral antigen(s). That is, in some embodiments the APCs are from (or are derived from cells obtained from) the same subject as the subject from which the population of cells with which they are to be co-cultured were obtained.

The use of polyclonal activated T cells (ATCs) as APCs and methods for preparing ATCs are described e.g. in Ngo et al., J Immunother. (2014) 37(4):193-203, incorporated by reference hereinabove. Briefly, ATCs can be generated by non-specifically activating T cells in vitro by stimulating PBMCs with agonist anti-CD3 and agonist anti-CD28 antibodies, in the presence of IL-2.

Dendritic cells may be generated according to methods well known in the art, e.g. as described in Ngo et al., J Immunother. (2014) 37(4):193-203. Dendritic cells may be prepared from monocytes which may be obtained by CD14 selection from PBMCs. The monocytes may be cultured in cell culture medium causing their differentiation to immature dendritic cells, which may comprise e.g. IL-4 and GM-CSF. Immature dendritic cells may be matured by culture in the presence of IL-6, IL-1β, TNFα, PGE2, GM-CSF and IL-4.

LCLs may be generated according to methods well known in the art, e.g. as described in Hui-Yuen et al., J Vis Exp (2011) 57: 3321, and Hussain and Mulherkar, Int J Mol Cell Med (2012) 1(2): 75-87, both hereby incorporated by reference in their entirety. Briefly, LCLs can be produced by incubation of PBMCs with concentrated cell culture supernatant of cells producing EBV, for example B95-8 cells, in the presence of cyclosporin A.

Artificial antigen presenting cells (aAPCs) include e.g. K562cs cells, which are engineered to express costimulatory molecules CD80, CD86, CD83 and 4-1BBL (described e.g. in Suhoski et al., Mol Ther. (2007) 15(5):981-8).

In some embodiments, a stimulation step comprises contacting PBMCs with peptide(s) corresponding to viral antigen(s). In some embodiments, a re-stimulation step comprises contacting immune cells specific for viral antigen(s) with APCs presenting peptide(s) corresponding to viral antigen(s). In some embodiments, a re-stimulation step comprises contacting immune cells specific for viral antigen(s) with ATCs presenting peptide(s) corresponding to viral antigen(s).

In some embodiments, the methods further employ agents for enhancing costimulation in stimulations and/or re-stimulations. Such agents include e.g. cells expressing costimulatory molecules (e.g. CD80, CD86, CD83 and/or 4-1BBL), such as e.g. LCLs or K562cs cells. In some embodiments the cells expressing costimulatory molecules are HLA-negative, EBV replication-incompetent LCLs, which are also referred to as “universal LCLs” or “uLCLs”. uLCLs are described e.g. in US 2018/0250379 A1.

Other examples of agents for enhancing costimulation include e.g. agonist antibodies specific for costimulatory receptors expressed by T cells (e.g. 4-1BB, CD28, OX40, ICOS, etc.), and costimulatory molecules capable of activating costimulatory receptors expressed by T cells (e.g. CD80, CD86, CD83, 4-1BBL, OX40L, ICOSL, etc.). Such agents may be provided e.g. immobilised on beads.

In some embodiments stimulations and/or re-stimulations according to the present disclosure employ uLCLs. ULCLs express EBV antigens and also express CD30, along with other costimulatory molecules. Thus uLCLs are useful for providing EBV antigenic stimulation, stimulation of CD30.CAR EBVSTs through the CAR via CD30, and also costimulation for the in vitro/ex vivo expansion of CD30.CAR EBVSTs.

In some embodiments, the uLCLs are employed as cells providing antigenic stimulation (e.g. EBV and/or CD30 stimulation). In some embodiments, the uLCLs are employed as cells providing costimulation. In some embodiments, the uLCLs are employed as cells providing antigenic stimulation and costimulation. In some embodiments, the uLCLs are irradiated (e.g. at 100 gray). In some embodiments, APCs presenting peptide(s) corresponding to viral antigen(s) are uLCLs.

In particular embodiments, the methods of the present disclosure comprise culturing immune cells specific for viral antigen(s) in the presence of uLCLs. In particular embodiments, the methods of the present disclosure comprise a restimulation step comprising culturing immune cells specific for viral antigen(s) in the presence of uLCLs. In some embodiments, uLCLs (e.g. irradiated uLCLs) may be employed in co-cultures with immune cells specific for viral antigen(s) at a ratio of immune cells specific 15 for viral antigen(s) to uLCLs between 1:1 and 1:10, e.g. one of 1:1.5, 1:2, 1:2.5, 1:3, 1:3.5, 1:4, 1:4.5, 1:5, 1:5.5, 1:6, 1:6.5, 1:7, 1:7.5 or 1:8. In some embodiments, uLCLs (e.g. irradiated uLCLs) may be employed in co-cultures with immune cells specific for viral antigen(s) at a ratio of immune cells specific for viral antigen(s) to uLCLs between 1:2 and 1:5, e.g. one of 1:2, 1:2.5, 1:3, 1:3.5, 1:4, 1:4.5 or 1:5. In some embodiments, the ratio of immune cells specific for viral antigen(s) to uLCLs is ˜1:3.

In particular embodiments, the methods of the present disclosure comprise culturing virus-specific immune cells comprising/expressing a CAR described herein (or comprising/expressing nucleic acid encoding such a CAR) in the presence of uLCLs. In particular embodiments, the methods of the present disclosure comprise a restimulation step comprising culturing virus-specific immune cells comprising/expressing a CAR described herein (or comprising/expressing nucleic acid encoding such a CAR) in the presence of uLCLs. In some embodiments, uLCLs (e.g. irradiated uLCLs) may be employed in co-cultures with virus-specific immune cells comprising/expressing a CAR described herein (or comprising/expressing nucleic acid encoding such a CAR) at a ratio of virus-specific immune cells comprising/expressing a CAR described herein (or comprising/expressing nucleic acid encoding such a CAR) to uLCLs between 1:1 and 1:10, e.g. one of 1:1.5, 1:2, 1:2.5, 1:3, 1:3.5, 1:4, 1:4.5, 1:5, 1:5.5, 1:6, 1:6.5, 1:7, 1:7.5 or 1:8. In some embodiments, uLCLs (e.g. irradiated uLCLs) may be employed in co-cultures with virus-specific immune cells comprising/expressing a CAR described herein (or comprising/expressing nucleic acid encoding such a CAR) at a ratio of virus-specific immune cells comprising/expressing a CAR described herein (or comprising/expressing nucleic acid encoding such a CAR) to uLCLs between 1:2 and 1:5, e.g. one of 1:2, 1:2.5, 1:3, 1:3.5, 1:4, 1:4.5 or 1:5. In some embodiments, the ratio of virus-specific immune cells comprising/expressing a CAR described herein (or comprising/expressing nucleic acid encoding such a CAR) to uLCLs is ˜1:3.

In some embodiments, a re-stimulation step comprises contacting immune cells specific for viral antigen(s) with ATCs presenting peptide(s) corresponding to viral antigen(s) in the presence of uLCLs. Contacting of populations of immune cells with peptide(s) corresponding to viral antigen(s), or APCs presenting peptide(s) corresponding to viral antigen(s), may be performed in the presence of one or more cytokines, to facilitate T cell activation and proliferation. In some embodiments stimulations are performed in the presence of one or more of IL-7, IL-15, IL-6, IL-12, IL-4, IL-2 and/or IL-21. It will be appreciated that the cytokines are added exogenously to the culture, and additional to cytokines that are produced by the cells in culture. In some embodiments the added cytokines are recombinantly-produced cytokines.

Accordingly, in some embodiments the methods involve culturing PBMCs that have been contacted with peptide(s) corresponding to viral antigen(s), or in the presence of APCs presenting peptide(s) corresponding to viral antigen(s), in the presence of one or more of IL-7, IL-15, IL-6, IL-12, IL-4, IL-2 and/or IL-21.

In some embodiments culture is in the presence of IL-7, IL-15, IL-6, IL-12, IL-4, IL-2 and/or IL-21. In some embodiments culture is in the presence of IL-7, IL-15, IL-8 and/or IL-12. In some embodiments culture is in the presence of IL-7 and/or IL-15.

In some embodiments the final concentration of IL-7 in the culture is 1-100 ng/ml, e.g. one of 2-50 ng/ml, 5-20 ng/ml or 7.5-15 ng/ml. In some embodiments the final concentration of IL-7 in the culture is about 10 ng/ml.

In some embodiments the final concentration of IL-15 in the culture is 1-100 ng/mL, e.g. one of 2-50 ng/mL, 5-20 ng/ml or 7.5-15 ng/ml. In some embodiments the final concentration of IL-15 in the culture is about 10 ng/ml. In some embodiments the final concentration of IL-15 in the culture is 10-1000 ng/mL, e.g. one of 20-500 ng/ml, 50-200 ng/ml or 75-150 ng/ml. In some embodiments the final concentration of IL-15 in the culture is about 100 ng/ml.

In some embodiments the final concentration of IL-6 in the culture is 10-1000 ng/ml, e.g. one of 20-500 ng/ml, 50-200 ng/ml or 75-150 ng/ml. In some embodiments the final concentration of IL-6 in the culture is about 100 ng/ml.

In some embodiments the final concentration of IL-12 in the culture is 1-100 ng/mL, e.g. one of 2-50 ng/mL, 5-20 ng/ml or 7.5-15 ng/ml. In some embodiments the final concentration of IL-12 in the culture is 10 ng/ml.

In some embodiments the final concentration of IL-7 is 1-100 ng/ml (e.g. one of 2-50 ng/ml, 5-20 ng/ml or 7.5-15 ng/ml, e.g. 10 ng/ml), and the final concentration of IL-15 is 1-100 ng/ml (e.g. one of 2-50 ng/ml, 5-20 ng/ml or 7.5-15 ng/ml, e.g. about 10 ng/ml).

In some embodiments the final concentration of IL-7 is 1-100 ng/ml (e.g. one of 2-50 ng/ml, 5-20 ng/ml or 7.5-15 ng/ml, e.g. 10 ng/ml), and the final concentration of IL-15 is 10-1000 ng/ml (e.g. one of 20-500 ng/ml, 50-200 ng/ml or 75-150 ng/mL, e.g. about 100 ng/ml).

In some embodiments the final concentration of IL-7 is 1-100 ng/ml (e.g. one of 2-50 ng/ml, 5-20 ng/ml or 7.5-15 ng/ml, e.g. 10 ng/ml), the final concentration of IL-6 is 10-1000 ng/ml (e.g. one of 20-500 ng/ml, 50-200 ng/mL or 75-150 ng/ml, e.g. about 100 ng/ml), the final concentration of IL-12 is 1-100 ng/ml (e.g. one of 2-50 ng/ml, 5-20 ng/ml or 7.5-15 ng/ml, e.g. 10 ng/ml), and the final concentration of IL-15 is 1-100 ng/ml (e.g. one of 2-50 ng/ml, 5-20 ng/ml or 7.5-15 ng/ml, e.g. 10 ng/ml).

In some embodiments the final concentration of IL-7 in a stimulation culture is 1-100 ng/ml (e.g. one of 2-50 ng/ml, 5-20 ng/ml or 7.5-15 ng/ml, e.g. 10 ng/mL), and the final concentration of IL-15 in a stimulation culture is 10-1000 ng/ml (e.g. one of 20-500 ng/ml, 50-200 ng/ml or 75-150 ng/ml, e.g. about 100 ng/ml).

In some embodiments the final concentration of IL-7 in a stimulation culture is 1-100 ng/ml (e.g. one of 2-50 ng/ml, 5-20 ng/ml or 7.5-15 ng/ml, e.g. 10 ng/mL), the final concentration of IL-8 in a stimulation culture is 10-1000 ng/ml (e.g. one of 20-500 ng/ml, 50-200 ng/ml or 75-150 ng/ml, e.g. about 100 ng/ml), the final concentration of IL-12 in a stimulation culture is 1-100 ng/ml (e.g. one of 2-50 ng/ml, 5-20 ng/ml or 7.5-15 ng/ml, e.g. 10 ng/ml), and the final concentration of IL-15 in a stimulation culture is 1-100 ng/ml (e.g. one of 2-50 ng/mL, 5-20 ng/ml or 7.5-15 ng/ml, e.g. 10 ng/ml).

In some embodiments the final concentration of IL-7 in a re-stimulation culture is 1-100 ng/ml (e.g. one of 2-50 ng/ml, 5-20 ng/ml or 7.5-15 ng/ml, e.g. 10 ng/ml), and the final concentration of IL-15 in a re-stimulation culture is 10-1000 ng/ml (e.g. one of 20-500 ng/ml, 50-200 ng/mL or 75-150 ng/mL, e.g. about 100 ng/ml).

Stimulations and re-stimulations according to the present disclosure typically involve co-culture of T cells and APCs for a period of time sufficient for APCs to stimulate the T cells, and for the T cells to undergo cell division.

In some embodiments, the methods involve culturing PBMCs that have been contacted with peptide(s) corresponding to viral antigen(s), or in the presence of APCs presenting peptide(s) corresponding to viral antigen(s), for a period of one of at least 1 hour, 6 hours, 12 hours, 24 hours, 48 hours, 72 hours, 4 days, 5 days, 6 days, or at least 7 days. In some embodiments, culture is for a period of 24 hours to 20 days, e.g. one of 48 hours to 14 days, 3 days to 12 days, 4 to 11 days, or 6 to 10 days or 7 to 9 days.

In some embodiments, the methods involve culturing the population of cells obtained by a stimulation step described herein that have been contacted with peptide(s) corresponding to viral antigen(s), or in the presence of APCs presenting peptide(s) corresponding to viral antigen(s), for a period of one of at least 1 hour, 6 hours, 12 hours, 24 hours, 48 hours, 72 hours, 4 days, 5 days, 6 days, or at least 7 days. In some embodiments, culture is for a period of 24 hours to 20 days, e.g. one of 48 hours to 14 days, 3 days to 12 days, 4 to 11 days, or 6 to 10 days or 7 to 9 days.

Stimulations and re-stimulations may be ended by separating the cells in culture from the media in which they have been cultured, or diluting the culture, e.g. by the addition of cell culture medium. In some embodiments, the methods comprise a step of collecting the cells at the end of the stimulation or re-stimulation culture. In some embodiments, a re-stimulation step may be established by adding cell culture medium (and any other additives as described herein) in an amount appropriate to achieve the desired percentages/concentrations of cell culture medium, conditioned media (and any additives) for the re-stimulation step.

At the end of the culture period of a given stimulation or re-stimulation step, the cells may be collected and separated from the cell culture supernatant. The cells may be collected by centrifugation, and the cell culture supernatant may be separated from the cell pellet. The cell pellet may then be re-suspended in cell culture medium, e.g. for a re-stimulation step. In some embodiments, the cells may undergo a washing step after collection. A washing step may comprise re-suspending the cell pellet in isotonic buffer such as phosphate-buffered saline (PBS), collecting the cells by centrifugation, and discarding the supernatant.

Methods for generating and/or expanding populations of immune cells specific for viral antigen(s) typically involve more than a single stimulation step. There is no upper limit to the number of stimulation steps which may be performed. In some embodiments the methods comprise more than 2, 3, 4 or 5 stimulation steps. In some embodiments, the methods comprise one of 2, 3, 4, 5, 6, 7, 8, 9, 10, 11, 12, 13, 14, or 15 stimulation steps. The stimulation steps in a method may be different to one another.

In some embodiments, the PBMCs employed in the methods are depleted of CD45RA-positive cells. That is, in some embodiments, the PBMCs are “CD45RA-positive cell-depleted PBMCs”, or are “CD45RA-negative PBMCs”. Depletion of CD45RA-positive cells is intended to reduce the number of NK cells and/or regulatory T cells in the populations of cells generated/expanded.

In some embodiments, the methods comprise a step of depleting PBMCs of CD45RA-positive cells, e.g. prior to a stimulation step. In some embodiments, the methods comprise a step of depleting the cells obtained by a stimulation step according to the present disclosure of CD45RA-positive cells, e.g. prior to a re-stimulation step. Depletion of CD45RA-positive cells can be achieved by any suitable method, such as by magnetic-activated cell sorting (MACS), for example using Miltenyi® Biotec columns and magnetic anti-CD45RA antibody-coated beads.

In some embodiments, the population of cells used to derive APCs employed in the methods is depleted of CD45RA-positive cells. That is, in some embodiments, the population of cells used to derive APCs is a “CD45RA-positive cell-depleted” or “CD45RA-negative” population. For example, in embodiments wherein ATCs are employed as APCs, the ATCs may be derived from a population of CD45RA-positive cell-depleted PBMCs, or from a population of CD45RA-negative PBMCs.

In some embodiments the methods further comprise modification of the immune cells specific for viral antigen(s) to increase IL-7-mediated signalling in the cells. IL-7-mediated signalling has been shown to increase the survival and anti-tumor activity of tumor-specific T cells—see e.g. in Shum et al., Cancer Discov. (2017) 7(11):1238-1247, and WO 2018/038945 A1.

In some embodiments, the methods further comprise introducing nucleic acid according to an embodiment described in WD 2018/038945 A1 (which is hereby incorporated by reference in its entirety) into the PBMCs or the immune cells specific for viral antigen(s). In some embodiments, the methods comprise introducing nucleic acid into the PBMCs or the immune cells specific for viral antigen(s), wherein the nucleic acid encodes a polypeptide for increasing STATS-mediated signalling within the cells.

In some embodiments the nucleic acid encodes a polypeptide comprising (i) a domain facilitating homo-dimerisation of the polypeptide, and (i) the intracellular domain of IL-7Rα.

In some embodiments, the domain facilitating homo-dimerisation of the polypeptide comprises or consists of an amino acid sequence providing for the formation of disulphide bonds between monomers of the polypeptide. In some embodiments the domain facilitating homo-dimerisation of the polypeptide comprises or consists of an amino acid sequence according to one of SEQ ID NOs:1 to 24 of WD 2018/038945 A1 (see e.g. paragraphs [0074] to [0076] of WD 2018/038945 A1).

The intracellular domain of IL-7Rα may comprise or consist of the amino acid sequence corresponding to positions 265 to 459 of UniProt: P16871-1, v1.

The nucleic acid may be introduced into the cells by methods well known in the art, such as transduction, transfection, electroporation, etc. In some embodiments the nucleic acid is introduced into the cells via transduction using a viral vector (e.g. a retroviral vector) comprising the nucleic acid.

In some embodiments, the method comprises transducing PBMCs or immune cells specific for EBV antigen(s) with a viral vector comprising nucleic acid encoding a polypeptide comprising (i) a domain facilitating homo-dimerisation of the polypeptide, and (ii) the intracellular domain of IL-7Rα.

Aspects and embodiments of the methods described herein comprise modifying an immune cell described herein (e.g. a virus-specific immune cell described herein) to express/comprise a CAR according to the present disclosure.

Aspects and embodiments of the methods described herein comprise modifying an immune cell described herein (e.g. a virus-specific immune cell described herein) to express/comprise nucleic acid encoding a CAR according to the present disclosure.

Such methods typically comprise introducing nucleic acid encoding a CAR into an immune cell. Immune cells (e.g. virus-specific immune cells) may be modified to comprise/express a CAR or nucleic acid encoding a CAR described herein according to methods that are well known to the skilled person. The methods generally comprise nucleic acid transfer for permanent (stable) or transient expression of the transferred nucleic acid.

Any suitable genetic engineering platform may be used to modify a cell according to the present disclosure. Suitable methods for modifying a cell include the use of genetic engineering platforms such as gammaretroviral vectors, lentiviral vectors, adenovirus vectors, DNA transfection, transposon-based gene delivery and RNA transfection, for example as described in Maus et al., Annu Rev Immunol (2014) 32:189-225, hereby incorporated by reference in its entirety. In some embodiments, modifying a cell to comprise a CAR or nucleic acid encoding a CAR comprises transducing a cell with a viral vector comprising nucleic acid encoding the CAR.

In some embodiments, the methods of the present disclosure employ a retrovirus encoding a CAR described herein.

Methods also include those described e.g. in Wang and RiviCre Mol Ther Oncolytics. (2016) 3:16015, which is hereby incorporated by reference in its entirety.

The methods generally comprise introducing a nucleic acid/plurality of nucleic acids encoding a vector/plurality of vectors comprising such nucleic acid(s), into a cell. In some embodiments, the methods additionally comprise culturing the cell under conditions suitable for expression of the nucleic acid(s) or vector(s) by the cell. In some embodiments, the methods are performed in vitro. Suitable methods for introducing nucleic acid(s)/vector(s) into cells include transduction, transfection and electroporation.

In some embodiments, introducing nucleic acid(s)/vector(s) into a cell comprises transduction, e.g. retroviral transduction. Accordingly, in some embodiments the nucleic acid(s) is/are comprised in a viral vector(s), or the vector(s) is/are a viral vector(s). Transduction of immune cells with viral vectors is described e.g. in Simmons and Alberola-IIa, Methods Mol Biol. (2016) 1323:99-108, which is hereby incorporated by reference in its entirety.

Agents may be employed in the methods of the present disclosure to enhance the efficiency of transduction. Hexadimethrine bromide (polybrene) is a cationic polymer which is commonly used to improve transduction, through neutralising charge repulsion between virions and sialic acid residues expressed on the cell surface. Other agents commonly used to enhance transduction include e.g. the poloxamer-based agents such as LentiBOOST (Sirion Biotech), Retronectin (Takara), Vectofusin (Miltenyi Biotech) and also SureENTRY (Qiagen) and ViraDuctin (Cell Biolabs).

In particular embodiments, the methods of the present disclosure employ Vectofusin-1 (Miltenyi Biotec Cat No. 170-076-165) in the transduction of cells with a vector/nucleic acid encoding a CAR described herein. In some embodiments, the methods comprise contacting retrovirus encoding a CAR described herein with Vectofusin-1, and contacting cells to be transduced with the retrovirus with the mixture comprising retrovirus and Vectofusin-1.

In some embodiments, the methods comprise centrifuging the cells into which it is desired to introduce nucleic acid encoding the CAR in the presence of cell culture medium comprising viral vector comprising the nucleic acid (referred to in the art as ‘spinfection’).

In some embodiments, the methods comprise introducing a nucleic acid or vector according to the present disclosure by electroporation, e.g. as described in Koh et al., Molecular Therapy—Nucleic Acids (2013) 2, e114, which is hereby incorporated by reference in its entirety.

In some embodiments, the methods further comprise purifying/isolating CAR-expressing and/or virus-specific immune cells, e.g. from other cells (e.g. cells which are not specific for the virus, and/or cells which do not express the CAR). Methods for purifying/isolating immune cells from heterogeneous populations of cells are well known in the art, and may employ e.g. FACS- or MACS-based methods for sorting populations of cells based on the expression of markers of the immune cells. In some embodiments the method is for purifying/isolating cells of a particular type, e.g. virus-specific T cells (e.g. virus-specific CD8+ T cells, virus-specific CTLs), or CAR-expressing virus-specific T cells (e.g. CAR-expressing virus-specific CD8+ T cells, CAR-expressing virus-specific CTLs).

The present disclosure also provides cells obtained or obtainable by the methods described herein, and populations thereof.

Compositions

The present disclosure further provides compositions comprising one or more (e.g. a population of) CAR-expressing virus-specific immune cells according to the present disclosure.

The cells described herein may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion.

Suitable formulations may comprise the cells in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments the composition is formulated for injection or infusion, e.g. into a blood vessel or tumor.

The present disclosure also provides methods for the production of pharmaceutically useful compositions, such methods of production may comprise one or more steps selected from: producing a cell described herein; isolating a cell described herein; and/or mixing a cell described herein with a pharmaceutically acceptable carrier, adjuvant, excipient or diluent.

For example, a further aspect the present disclosure relates to a method of formulating or producing a medicament or pharmaceutical composition for use in the treatment of a disease/condition (e.g. a cancer), the method comprising formulating a pharmaceutical composition or medicament by mixing a cell described herein with a pharmaceutically acceptable carrier, adjuvant, excipient or diluent.

MHC Variation and Matching

MHC class I molecules are non-covalent heterodimers of an alpha (α) chain and a beta (β)2-microglobulin (B2M). The α-chain has three domains designated α1, α2 and α3. The α1 and α2 domains together form the groove to which the peptide presented by the MHC class I molecule binds, to form the peptide:MHC complex. In humans, MHC class I α-chains are encoded by human leukocyte antigen (HLA) genes. There are three major HLA gene loci (HLA-A, HLA-B and HLA-C) and three minor loci (HLA-E, HLA-F and HLA-G).

MHC class I α-chains are polymorphic, and different α-chains are capable of binding and presenting different peptides. Genes encoding MHC class I α polypeptides are highly variable, with the result that cells from different subjects often express different MHC class I molecules.

This variability has implications for organ transplantation and adoptive transfer of cells between individuals. The immune system of a recipient of a transplant or adoptively transferred cells recognises the non-self MHC molecules as foreign, triggering an immune response directed against the transplant or adoptively transferred cells, which can lead to graft rejection. Alternatively, cells amongst the population of cells/tissue/organ to be transplanted may contain immune cells which recognise the recipient's MHC molecules as foreign, triggering an immune response directed against recipient tissues, which can lead to graft versus host disease (GVHD).

Alloreactive T cells comprise TCRs capable of recognising non-self MHC molecules (i.e. allogeneic MHC), and initiating an immune response thereto. Alloreactive T cells may display one or more of the following properties in response to a cell expressing a non-self MHC molecule: cell proliferation, growth factor (e.g. IL-2) expression, cytotoxic/effector factor (e.g. IFNγ, granzyme, perforin, granulysin, CD107a, TNFα, FASL) expression and/or cytotoxic activity.

“Alloreactivity” and an “alloreactive immune response” as used herein refers to an immune response directed against a cell/tissue/organ which is genetically non-identical to the effector immune cell. An effector immune cell may display alloreactivity or an alloreactive immune response to cells—or tissues/organs comprising cells—expressing non-self MHC/HLA molecules (i.e. MHC/HLA molecules which are non-identical to the MHC/HLA molecules encoded by the effector immune cells).

“MHC mismatched” and “HLA mismatched” subjects as referred to herein are subjects having MHC/HLA genes encoding non-identical MHC/HLA molecules. In some embodiments the MHC mismatched or HLA mismatched subjects have MHC/HLA genes encoding non-identical MHC class I α and/or MHC class II molecules. “MHC matched” and “HLA matched” subjects as referred to herein are subjects having MHC/HLA genes encoding identical MHC/HLA molecules. In some embodiments the MHC matched or HLA matched subjects have MHC/HLA genes encoding identical MHC class I α and/or MHC class II molecules.

Where a cell/tissue/organ is referred to herein as being allogeneic with respect to a reference subject/treatment, the cell/tissue/organ is from obtained/derived from cells/tissue/organ of a subject other than the reference subject. In some embodiments, allogeneic material comprises MHC/HLA genes encoding MHC/HLA molecules (e.g. MHC class I α and/or MHC class II molecules) which are non-identical to the MHC/HLA molecules (e.g. MHC class I α and/or MHC class II molecules) encoded by the MHC/HLA genes of the reference subject.

Where a cell/tissue/organ is referred to herein as being allogeneic with respect to a treatment, the cell/tissue/organ is from obtained/derived from cells/tissue/organ of a subject other than the subject to be treated. In some embodiments, allogeneic material comprises MHC/HLA genes encoding MHC/HLA molecules (e.g. MHC class I α and/or MHC class II molecules) which are non-identical to the MHC/HLA molecules (e.g. MHC class I α and/or MHC class II molecules) encoded by the MHC/HLA genes of the subject to be treated.

Where a cell/tissue/organ is referred to herein as being autologous with respect to a reference subject, the cell/tissue/organ is from obtained/derived from cells/tissue/organ of the reference subject. Where a cell/tissue/organ is referred to herein as being autogeneic with respect to a reference subject, cell/tissue/organ is genetically identical to the reference subject, or derived/obtained from a genetically identical subject. Where a cell/tissue/organ is referred to herein as being autologous in the context of a treatment of a subject (e.g. treatment by administration to a subject of autologous cells), the cell/tissue/organ is obtained/derived from cells/tissue/organ of the subject to be treated. Where a cell/tissue/organ is referred to herein as being autogeneic in the context of a treatment of a subject, the cell/tissue/organ is genetically identical to the subject to be treated, or derived/obtained from a genetically identical subject. Autologous and autogeneic cell/tissue/organs comprise MHC/HLA genes encoding MHC/HLA molecules (e.g. MHC class I α and/or MHC class II molecules) which are identical to the MHC/HLA molecules (e.g. MHC class I α and/or MHC class II molecules) encoded by the MHC/HLA genes of the reference subject.

Where a cell/tissue/organ is referred to herein as being allogeneic with respect to a reference subject, cell/tissue/organ is genetically non-identical to the reference subject, or derived/obtained from a genetically non-identical subject. Where a cell/tissue/organ is referred to herein as being allogeneic in the context of a treatment of a subject, the cell/tissue/organ is genetically non-identical to the subject to be treated, or derived/obtained from a genetically non-identical subject. Allogeneic cell/tissue/organs may comprise MHC/HLA genes encoding MHC/HLA molecules (e.g. MHC class I α and/or MHC class II molecules) which are non-identical to the MHC/HLA molecules (e.g. MHC class I α and/or MHC class II molecules) encoded by the MHC/HLA genes of the reference subject.

In some embodiments, immune cells specific for a virus expressing/comprising a CAR described herein (or expressing/comprising nucleic acid encoding such a CAR) to be administered to a subject in accordance with the methods of the present disclosure are selected based on the HLA/MHC profile of the subject to be treated.

In some embodiments, the cells to be administered to the subject are selected based on their being HLA/MHC matched with respect to the subject. In some embodiments, the cells to be administered to the subject are selected based on their being a near or complete HLA/MHC match with respect to the subject.

As used herein, HLA/MHC alleles may be determined to ‘match’ when they encode polypeptides having the same amino acid sequence. That is, the ‘match’ is determined at the protein level, irrespective of the possible presence of synonymous differences in the nucleotide sequences encoding the polypeptides and/or differences in the non-coding regions.

Cells which are ‘HLA matched’ with respect to a reference subject may be: (i) an 8/8 match across HLA-A, -B, -C, and -DRB1; or (ii) a 10/10 match across HLA-A, -B, -C, -DRB1 and -DQB1; or (iii) a 12/12 match across HLA-A, -B, -C, -DRB1, -DQB1 and -DPB1. Cells which are ‘a near or complete HLA match’ with respect to a reference subject may be: (i) a 24/8 (i.e. 4/8, 5/8, 6/8, 7/8 or 8/8) match across HLA-A, -B, —C, and -DRB1; or (ii) a 25/10 (i.e. 5/10, 6/10, 7/10, 8/10, 9/10 or 10/10) match across HLA-A, -B, -C, -DRB1 and -DQB1; or (iii) a 26/12 (i.e. 6/12, 7/12, 8/12, 9/12, 10/12, 11/12 or 12/12) match across HLA-A, -B, -C, -DRB1, -DQB1 and -DPB1.

Administration of cells to a subject which are a near or complete HLA match (irrespective of their being of allogeneic origin) can be advantageous, especially in the context of administration of immune cells specific for a virus expressing/comprising a CAR described herein (or expressing/comprising nucleic acid encoding such a CAR) for the treatment of a disease/condition caused by, or associated with, infection with the virus for which the immune cells are specific. In such instances, presentation of viral antigens by cells of the host to the administered cells (through their native TCRs) would be expected to increase their activation, proliferation and survival in vivo, and consequently improve their therapeutic efficacy.

Methods Using the CAR-Expressing, Virus-Specific Immune Cells

The CAR-expressing, virus-specific immune cells described herein (e.g. the CD30-specific CAR-expressing EBV-specific T cells (CD30.CAR EBVSTs) described herein) find use in therapeutic and/or prophylactic methods.

A method for treating/preventing a disease/condition in a subject is provided, comprising administering virus-specific immune cells expressing a CAR according to the present disclosure to a subject.

Also provided are virus-specific immune cells expressing a CAR according to the present disclosure for use in a method of medical treatment/prophylaxis. Also provided are virus-specific immune cells expressing a CAR according to the present disclosure for use in a method for treating/preventing a disease/condition. Also provided is the use of virus-specific immune cells expressing a CAR according to the present disclosure in the manufacture of a medicament for use in a method for treating/preventing a disease/condition.

It will be appreciated that the methods generally comprise administering a population of virus-specific immune cells expressing a CAR according to the present disclosure to a subject. In some embodiments, virus-specific immune cells expressing a CAR according to the present disclosure may be administered in the form of a pharmaceutical composition comprising such cells.

In particular, use of virus-specific immune cells expressing a CAR according to the present disclosure in methods to treat/prevent diseases/conditions by adoptive cell transfer (ACT) is contemplated.

The virus-specific immune cells expressing a CAR according to the present disclosure are particularly useful in methods for treating diseases/conditions by allotransplantation.

As used herein, “allotransplantation” refers to the transplantation to a recipient subject of cells, tissues or organs which are genetically non-identical to the recipient subject. The cells, tissues or organs may be from, or may be derived from, cells, tissues or organs of a donor subject that is genetically non-identical to the recipient subject. Allotransplantation is distinct from autotransplantation, which refers to the transplantation of cells, tissues or organs which are from/derived from a donor subject genetically identical to the recipient subject.

It will be appreciated that adoptive transfer of allogeneic immune cells is a form of allotransplantation. In some embodiments, the CAR-expressing virus-specific immune cells are used as therapeutic/prophylactic agents in methods for treating/preventing diseases/conditions by allotransplantation.

Administration of the CAR-expressing virus-specific immune cells and compositions of present disclosure is preferably in a “therapeutically effective” or “prophylactically effective” amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Multiple doses may be provided. Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, or more hours or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

In some embodiments, the treatment may further comprise other therapeutic or prophylactic intervention, e.g. chemotherapy, immunotherapy, radiotherapy, surgery, vaccination and/or hormone therapy. Such other therapeutic or prophylactic intervention may occur before, during and/or after the therapies encompassed by the disclosure, and the deliveries of the other therapeutic or prophylactic interventions may occur via different administration routes as the therapies of the disclosure.

Administration may be alone or in combination with other treatments, either simultaneously or sequentially dependent upon the condition to be treated. The CAR-expressing virus-specific immune cells and compositions described herein may be administered simultaneously or sequentially with another therapeutic intervention.

Simultaneous administration refers to administration of two or more therapeutic interventions together, for example as a pharmaceutical composition containing both active agents (i.e. in a combined preparation), or immediately after one another and optionally via the same route of administration, e.g. to the same artery, vein or other blood vessel.

Sequential administration refers to administration of one therapeutic intervention followed after a given time interval by separate administration of one or more further therapeutic interventions. It is not required that the therapies are administered by the same route, although this is the case in some embodiments. The time interval may be any time interval.

Adoptive cell transfer generally refers to a process by which cells (e.g. immune cells) are obtained from a subject, typically by drawing a blood sample from which the cells are isolated. The cells are then typically modified and/or expanded, and then administered either to the same subject (in the case of adoptive transfer of autologous/autogeneic cells) or to a different subject (in the case of adoptive transfer of allogeneic cells). The treatment is typically aimed at providing a population of cells with certain desired characteristics to a subject, or increasing the frequency of such cells with such characteristics in that subject. Adoptive transfer may be performed with the aim of introducing a cell or population of cells into a subject, and/or increasing the frequency of a cell or population of cells in a subject.

Adoptive transfer of immune cells is described, for example, in Kalos and June (2013), Immunity 39(1): 49-60, and Davis et al. (2015), Cancer J. 21(6): 486-491, both of which are hereby incorporated by reference in their entirety. The skilled person is able to determine appropriate reagents and procedures for adoptive transfer of cells according to the present disclosure, for example by reference to Dai et al., 2016 J Nat Cancer Inst 108(7): djv439, which is incorporated by reference in its entirety.

The present disclosure provides methods comprising administering a virus-specific immune cell comprising/expressing a CAR according to the present disclosure, or a virus-specific immune cell comprising/expressing nucleic acid encoding a CAR according to the present disclosure, to a subject.

In some embodiments, the methods comprise generating an immune cell specific for a virus, or generating/expanding a population of immune cells specific for a virus. In some embodiments, the methods comprise modifying an immune cell specific for a virus to comprise/express a CAR according to the present disclosure. In some embodiments, the methods comprise modifying an immune cell specific for a virus to comprise/express nucleic acid encoding a CAR according to the present disclosure.

In some embodiments, the methods comprise administering to a subject an immune cell specific for a virus modified to express/comprise a CAR according to the present disclosure (or modified to express/comprise a nucleic acid encoding such a CAR).

In some embodiments, the methods comprise:
(a) modifying an immune cell specific for a virus to express or comprise a CAR according to the present disclosure, or to express or comprise nucleic acid encoding a CAR according to the present disclosure, and
(b) administering the immune cell specific for a virus modified to express or comprise a CAR according to the present disclosure, or modified to express or comprise a nucleic acid encoding a CAR according to the present disclosure, to a subject.

In some embodiments, the methods comprise:
(a) isolating or obtaining immune cells specific for a virus;
(b) modifying an immune cell specific for a virus to express or comprise a CAR according to the present disclosure, or to express or comprise nucleic acid encoding a CAR according to the present disclosure, and
(c) administering the immune cell specific for a virus modified to express or comprise a CAR according to the present disclosure, or modified to express or comprise a nucleic acid encoding a CAR according to the present disclosure, to a subject.

In some embodiments, the methods comprise:
(a) isolating immune cells (e.g. PBMCs) from a subject;
(b) generating/expanding a population of immune cells specific for a virus;
(c) modifying an immune cell specific for a virus to express or comprise a CAR according to the present disclosure, or to express or comprise nucleic acid encoding a CAR according to the present disclosure, and
(d) administering the immune cell specific for a virus modified to express or comprise a CAR according to the present disclosure, or modified to express or comprise a nucleic acid encoding a CAR according to the present disclosure, to a subject.

In some embodiments, the methods comprise administering to a subject an EBV-specific immune cell modified to express or comprise a CD30-specific CAR according to the present disclosure, or modified to express or comprise a nucleic acid encoding a CD30-specific CAR according to the present disclosure.

In some embodiments, the methods comprise:
(a) modifying an EBV-specific immune cell to express or comprise a CD30-specific CAR according to the present disclosure, or to express or comprise nucleic acid encoding a CD30-specific CAR according to the present disclosure, and
(b) administering the EBV-specific immune cell modified to express or comprise a CD30-specific CAR according to the present disclosure, or modified to express or comprise a nucleic acid encoding a CD30-specific CAR according to the present disclosure, to a subject.

In some embodiments, the methods comprise:
(a) isolating or obtaining EBV-specific immune cells;
(b) modifying an EBV-specific immune cell to express or comprise a CD30-specific CAR according to the present disclosure, or to express or comprise nucleic acid encoding a CD30-specific CAR according to the present disclosure, and
(c) administering the EBV-specific immune cell modified to express or comprise a CD30-specific CAR according to the present disclosure, or modified to express or comprise a nucleic acid encoding a CD30-specific CAR according to the present disclosure, to a subject.

In some embodiments, the methods comprise:
(a) isolating immune cells (e.g. PBMCs) from a subject;
(b) generating/expanding a population of EBV-specific immune cells;
(c) modifying an EBV-specific immune cell to express or comprise a CD30-specific CAR according to the present disclosure, or to express or comprise nucleic acid encoding a CD30-specific CAR according to the present disclosure, and
(d) administering the EBV-specific immune cell modified to express or comprise a CD30-specific CAR according to the present disclosure, or modified to express or comprise a nucleic acid encoding a CD30-specific CAR according to the present disclosure, to a subject.

In some embodiments, the subject from which the immune cells (e.g. PBMCs) are isolated is the same subject to which cells are administered (i.e., adoptive transfer may be of autologous/autogeneic cells). In some embodiments, the subject from which the immune cells (e.g. PBMCs) are isolated is a different subject to the subject to which cells are administered (i.e., adoptive transfer may be of allogeneic cells).

In some embodiments the methods may comprise one or more of:
obtaining a blood sample from a subject;
isolating immune cells (e.g. PBMCs) from a blood sample which has been obtained from a subject;
generating/expanding a population of immune cells specific for a virus (e.g. by culturing PBMCs in the presence of cells (e.g. APCs) comprising/expressing antigen(s)/peptide(s) of the virus, or by culturing PBMCs in the presence of cells (e.g. APCs) infected with the virus);
culturing the immune cells specific for a virus in in vitro or ex vivo cell culture;
modifying an immune cell specific for a virus to express or comprise a CAR according to the present disclosure, or to express or comprise a nucleic acid encoding a CAR according to the present disclosure (e.g. by transduction with a viral vector encoding such CAR, or a viral vector comprising such nucleic acid);
culturing immune cells specific for a virus expressing/comprising a CAR according to the present disclosure, or expressing/comprising a nucleic acid encoding a CAR according to the present disclosure in in vitro or ex vivo cell culture;
collecting/isolating immune cells specific for a virus expressing/comprising a CAR according to the present disclosure, or expressing/comprising a nucleic acid encoding a CAR according to the present disclosure;
formulating immune cells specific for a virus expressing/comprising a CAR according to the present disclosure, or a nucleic acid encoding a CAR according to the present disclosure to a pharmaceutical composition, e.g. by mixing the cells with a pharmaceutically acceptable adjuvant, diluent, or carrier;
administering immune cells specific for a virus expressing/comprising a CAR according to the present disclosure, or expressing/comprising a nucleic acid encoding a CAR according to the present disclosure, or a pharmaceutical composition comprising such cells, to a subject.

In some embodiments, the methods may additionally comprise treating the cells or subject to induce/enhance expression of CAR and/or to induce/enhance proliferation or survival of virus-specific immune cells comprising/expressing the CAR.

The therapeutic and/or prophylactic methods may be effective to reduce the development/progression of a disease/condition, alleviate the symptoms of a disease/condition, or reduce the pathology of a disease/condition. The methods may be effective to prevent progression of the disease/condition, e.g. to prevent worsening of, or to slow the rate of development of, the disease/condition. In some embodiments the methods may lead to an improvement in the disease/condition, e.g. a reduction in the severity of symptoms of the disease/condition, or a reduction in some other correlate of the severity/activity of the disease/condition. In some embodiments the methods may prevent development of the disease/condition to a later stage (e.g. a chronic stage or metastasis).

It will be appreciated that the therapeutic and prophylactic utility of the CAR-expressing virus-specific immune cells according to the present disclosure extends to the treatment/prevention of any disease/condition that would derive therapeutic or prophylactic benefit from a reduction in the number/activity of cells expressing/overexpressing the target antigen of the CAR, and/or the number/activity of cells infected with the virus.

In some embodiments, the disease/condition to be treated/prevented in accordance with the present disclosure is a disease/condition in which the virus for which the immune cells are specific is pathologically implicated. That is, in some embodiments the disease/condition is a disease/condition which is caused or exacerbated by infection with the virus, a disease/condition for which infection with the virus is a risk factor and/or a disease/condition for which infection with the virus is positively associated with onset, development, progression, and/or severity of the disease/condition.

In some embodiments, the disease/condition to be treated/prevented in accordance with the present disclosure in which the target antigen for the CAR is pathologically implicated. That is, in some embodiments the disease/condition is a disease/condition which is caused or exacerbated by the expression/overexpression of the target antigen, a disease/condition for which expression/overexpression of the target antigen is a risk factor and/or a disease/condition for which expression/overexpression of the target antigen is positively associated with onset, development, progression, severity of the disease/condition.

The disease/condition may be a disease/condition in which CD30 or cells expressing/overexpressing CD30 are pathologically implicated, e.g. a disease/condition in which cells expressing/overexpressing CD30 are positively associated with the onset, development or progression of the disease/condition, and/or severity of one or more symptoms of the disease/condition, or for which CD30 expression/overexpression is a risk factor for the onset, development or progression of the disease/condition.

The disease/condition to be treated/prevented in accordance with the present disclosure may be a disease/condition characterised by EBV infection. For example, the disease/condition may be a disease/condition in which EBV or cells infected with EBV are pathologically implicated, e.g. a disease/condition in which EBV infection is positively associated with the onset, development or progression of the disease/condition, and/or severity of one or more symptoms of the disease/condition, or for which EBV infection is a risk factor for the onset, development or progression of the disease/condition.

The treatment may be aimed at one or more of reducing the viral load, reducing the number/proportion of virus-positive cells (e.g. EBV-positive cells), reducing the number/proportion of cells expressing/overexpressing the target antigen of the CAR (e.g. CD30-expressing cells), reducing the activity of virus-positive cells (e.g. EBV-positive cells), reducing the activity of cells expressing/overexpressing the target antigen of the CAR (e.g. CD30-expressing cells), delaying/preventing the onset/progression of symptoms of the disease/condition, reducing the severity of symptoms of the disease/condition, reducing the survival/growth of virus-positive cells (e.g. EBV-positive cells), reducing the survival/growth of cells expressing/overexpressing the target antigen of the CAR (e.g. CD30-expressing cells), or increasing survival of the subject.

In some embodiments, a subject may be selected for treatment described herein based on the detection of the virus (e.g. EBV), cells infected with the virus (e.g. EBV), or cells expressing/overexpressing the target antigen of the CAR (e.g. CD30) e.g. in the periphery, or in an organ/tissue which is affected by the disease/condition (e.g. an organ/tissue in which the symptoms of the disease/condition manifest), or by the detection of virus-positive cancer cells (e.g. EBV-positive cancer cells) or the detection of cancer cells expressing/overexpressing the target antigen of the CAR (e.g. CD30). The disease/condition may affect any tissue or organ or organ system. In some embodiments the disease/condition may affect several tissues/organs/organ systems.

In some embodiments a subject may be selected for therapy/prophylaxis in accordance with the present disclosure based on determination that the subject is infected with EBV or comprises cells infected with EBV. In some embodiments a subject may be selected for therapy/prophylaxis in accordance with the present disclosure based on determination that the subject comprises cells expressing/overexpressing CD30, e.g. CD30-expressing/overexpressing cancer cells.

In some embodiments, a subject is administered lymphodepleting chemotherapy prior to administration of immune cells specific for a virus expressing/comprising a CAR described herein (or expressing/comprising nucleic acid encoding such a CAR).

That is, in some embodiments, methods of treating/preventing a disease/condition in accordance with the present disclosure comprise: (i) administering a lymphodepleting chemotherapy to a subject, and (ii) subsequently administering an immune cell specific for a virus expressing/comprising a CAR according to the present disclosure, or expressing/comprising a nucleic acid encoding a CAR according to the present disclosure.

As used herein, “lymphodepleting chemotherapy” refers to treatment with a chemotherapeutic agent which results in depletion of lymphocytes (e.g. T cells, B cells, NK cells, NKT cells or innate lymphoid cell (ILCs), or precursors thereof) within the subject to which the treatment is administered. A “lymphodepleting chemotherapeutic agent” refers to a chemotherapeutic agent which results in depletion of lymphocytes.

Lymphodepleting chemotherapy and its use in methods of treatment by adoptive cell transfer are described e.g. in Klebanoff et al., Trends Immunol. (2005) 26(2):111-7 and Muranski et al., Nat Clin Pract Oncol. (2006) (12):668-81, both of which are hereby incorporated by reference in their entirety. The aim of lymphodepleting chemotherapy is to deplete the recipient subject's endogenous lymphocyte population.

In the context of treatment of disease by adoptive transfer of immune cells, lymphodepleting chemotherapy is typically administered prior to adoptive cell transfer, to condition the recipient subject to receive the adoptively transferred cells. Lymphodepleting chemotherapy is thought to promote the persistence and activity of adoptively transferred cells by creating a permissive environment, e.g. through elimination of cells expressing immunosuppressive cytokines, and creating the ‘lymphoid space’ required for expansion and activity of adoptively transferred lymphoid cells.

Chemotherapeutic agents commonly used in lymphodepleting chemotherapy include e.g. fludarabine, cyclophosphamide, bedamustine and pentostatin.

Aspects and embodiments of the present disclosure are particularly concerned with lymphodepleting chemotherapy comprising administration of fludarabine and/or cyclophosphamide. In particular embodiments, lymphodepleting chemotherapy according to the present disclosure comprises administration of fludarabine and cyclophosphamide.

Fludarabine is a purine analog that inhibits DNA synthesis by interfering with ribonucleotide reductase and DNA polymerase. It is often employed as a chemotherapeutic agent for the treatment of leukemia (particularly chronic lymphocytic leukemia, acute myeloid leukemia, acute lymphocytic leukemia) and lymphoma (particularly non-Hodgkin's Lymphoma). Fludarabine may be administered intravenously or orally.

Cyclophosphamide is an alkylating agent which causes irreversible intra-strand and inter-strand cross-links between DNA bases. It is often employed as a chemotherapeutic agent for the treatment of cancers including lymphomas, leukemia and multiple myeloma. Cyclophosphamide may be administered intravenously or orally.

A course of lymphodepleting chemotherapy in accordance with the present disclosure may comprise multiple administrations of one or more chemotherapeutic agents. A course of lymphodepleting chemotherapy may comprise administering fludarabine and cyclophosphamide at a dose described herein, and for a number of days described herein. By way of illustration, a course of lymphodepleting chemotherapy may comprise administering fludarabine at a dose of 30 mg/m² per day for 3 consecutive days, and administering cyclophosphamide at a dose of 500 mg/m² per day for 3 consecutive days.

The day of administration of the final dose of a chemotherapeutic agent in accordance with a course of lymphodepleting chemotherapy may be considered to be the day of completion of the course of lymphodepleting chemotherapy.

In some embodiments, fludarabine is administered at a dose of 5 to 100 mg/m² per day, e.g. one of 15 to 90 mg/m² per day, 15 to 80 mg/m² per day, 15 to 70 mg/m² per day, 15 to 60 mg/m² per day, 15 to 50 mg/m² per day, 10 to 40 mg/m² per day, 5 to 60 mg/m² per day, 10 to 60 mg/m² per day, 15 to 60 mg/m² per day, 20 to 60 mg/m² per day or 25 to 60 mg/m² per day. In some embodiments, fludarabine is administered at a dose of 20 to 40 mg/m² per day, e.g. 25 to 35 mg/m² per day, e.g. about 30 mg/m² per day.

In some embodiments fludarabine is administered at a dose according to the preceding paragraph for more than one day and fewer than 14 consecutive days. In some embodiments, fludarabine is administered at a dose according to the preceding paragraph for one of 2 to 14, e.g. 2 to 13, 2 to 12, 2 to 11, 2 to 10, 2 to 9, 2 to 8, 2 to 7, 2 to 6, 2 to 5 or 2 to 4 consecutive days. In some embodiments, fludarabine is administered at a dose according to the preceding paragraph for 2 to 6 consecutive days, e.g. 2 to 4 consecutive days, e.g. 3 consecutive days.

In some embodiments fludarabine is administered at a dose of 15 to 60 mg/m² per day, for 2 to 6 consecutive days, e.g. at a dose of 30 mg/m² per day, for 3 consecutive days.

In some embodiments, cyclophosphamide is administered at a dose of 50 to 1000 mg/m² per day, e.g. one of 100 to 900 mg/m² per day, 150 to 850 mg/m² per day, 200 to 800 mg/m² per day, 250 to 750 mg/m² per day, 300 to 700 mg/m² per day, 350 to 650 mg/m² per day, 400 to 600 mg/m² per day or 450 to 550 mg/m² per day. In some embodiments, cyclophosphamide is administered at a dose of 400 to 600 mg/m² per day, e.g. 450 to 550 mg/m² per day, e.g. about 500 mg/m² per day.

In some embodiments cyclophosphamide is administered at a dose according to the preceding paragraph for more than one day and fewer than 14 consecutive days. In some embodiments, cyclophosphamide is administered at a dose according to the preceding paragraph for one of 2 to 14, e.g. 2 to 13, 2 to 12, 2 to 11, 2 to 10, 2 to 9, 2 to 8, 2 to 7, 2 to 6, 2 to 5 or 2 to 4 consecutive days. In some embodiments, cyclophosphamide is administered at a dose according to the preceding paragraph for 2 to 6 consecutive days, e.g. 2 to 4 consecutive days, e.g. 3 consecutive days.

In some embodiments cyclophosphamide is administered at a dose of 400 to 600 mg/m² per day, for 2 to 6 consecutive days, e.g. at a dose of 500 mg/m² per day, for 3 consecutive days.

In some embodiments, fludarabine and cyclophosphamide may be administered simultaneously or sequentially. Simultaneous administration refers to administration together, for example as a pharmaceutical composition containing both agents (i.e. in a combined preparation), or immediately after one another, and optionally via the same route of administration, e.g. to the same artery, vein or other blood vessel. Sequential administration refers to administration of one of the agents followed after a given time interval by separate administration of the other agent. It is not required that the agents are administered by the same route, although this is the case in some embodiments.

In some embodiments of courses of lymphodepleting chemotherapy in accordance with the present disclosure, fludarabine and cyclophosphamide are administered on the same day or days. By way of illustration, in the example of a course of lymphodepleting chemotherapy comprising administering fludarabine at a dose of 30 mg/m² per day for 3 consecutive days, and administering cyclophosphamide at a dose of 500 mg/m² per day for 3 consecutive days, the fludarabine and cyclophosphamide may be administered on the same 3 consecutive days. In such an example, the course of lymphodepleting chemotherapy may be said to be completed on the final day of the 3 consecutive days on which fludarabine and cyclophosphamide are administered to the subject.

In some embodiments, immune cells specific for a virus expressing/comprising a CAR described herein (or expressing/comprising nucleic acid encoding such a CAR) are administered to a subject within a specified period of time following completion of a course of lymphodepleting chemotherapy.

In some embodiments, immune cells specific for a virus expressing/comprising a CAR described herein (or expressing/comprising nucleic acid encoding such a CAR), are administered to a subject within 1 to 28 days, e.g. one of 1 to 21 days, 1 to 14 days, 1 to 7 days, 2 to 7 days, 2 to 5 days, or 3 to 5 days of completion of a course of lymphodepleting chemotherapy described herein. In some embodiments, immune cells specific for a virus expressing/comprising a CAR described herein (or expressing/comprising nucleic acid encoding such a CAR), are administered to a subject within 2 to 14 days (e.g. within 3 to 5 days) of completion of a course of lymphodepleting chemotherapy described herein.

In some embodiments, immune cells specific for a virus expressing/comprising a CAR described herein (or expressing/comprising nucleic acid encoding such a CAR) are administered at a dose of 1×10⁷ cells/m² to 1×10⁹ cells/m², e.g. one of 2×10⁷ cells/m² to 1×10⁹ cells cells/m², 2.5×10⁷ cells/m² to 8×10⁸ cells cells/m², 3×10⁷ cells/m² to 6×10⁸ cells cells/m², or 4×10⁷ cells/in to 4×10⁸ cells cells/m².

In some embodiments, immune cells specific for a virus expressing/comprising a CAR described herein (or expressing/comprising nucleic acid encoding such a CAR) are administered at a dose of 4×10⁷ cells/m², 1×10⁸ cells/m² or 4×10⁸ cells/m².

Administration of immune cells specific for a virus expressing/comprising a CAR described herein (or expressing/comprising nucleic acid encoding such a CAR) may be administered by intravenous infusion. Administration may be in a volume between 1 and 50 ml, and may be performed over a period of 1 to 10 min.

In some embodiments, the disease to be treated/prevented in accordance with the present disclosure is a cancer.

Cancer may refer to any unwanted cell proliferation (or any disease manifesting itself by unwanted cell proliferation), neoplasm or tumor. The cancer may be benign or malignant and may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, and/or white blood cells.

Tumors may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, hematologic cancer and sarcoma.

In some embodiments the cancer is selected from the group consisting of: a solid cancer, a hematological cancer, gastric cancer (e.g. gastric carcinoma, gastric adenocarcinoma, gastrointestinal adenocarcinoma), liver cancer (hepatocellular carcinoma, cholangiocarcinoma), head and neck cancer (e.g. head and neck squamous cell carcinoma), oral cavity cancer (e.g. oropharyngeal cancer (e.g. oropharyngeal carcinoma), oral cancer, laryngeal cancer, nasopharyngeal carcinoma, oesophageal cancer), colorectal cancer (e.g. colorectal carcinoma), colon cancer, colon carcinoma, cervical carcinoma, prostate cancer, lung cancer (e.g. NSCLC, small cell lung cancer, lung adenocarcinoma, squamous lung cell carcinoma), bladder cancer, urothelial carcinoma, skin cancer (e.g. melanoma, advanced melanoma), renal cell cancer (e.g. renal cell carcinoma), ovarian cancer (e.g. ovarian carcinoma), mesothelioma, breast cancer, brain cancer (e.g. glioblastoma), prostate cancer, pancreatic cancer, a myeloid hematologic malignancy, a lymphoblastic hematologic malignancy, myelodysplastic syndrome (MDS), acute myeloid leukemia (AML), chronic myeloid leukemia (CML), acute lymphoblastic leukemia (ALL), lymphoma, non-Hodgkin's lymphoma (NHL), thymoma or multiple myeloma (MM).

In some embodiments the cancer is a cancer in which the virus for which the immune cells are specific is pathologically implicated. That is, in some embodiments the cancer is a cancer which is caused or exacerbated by infection with the virus, a cancer for which infection with the virus is a risk factor and/or a cancer for which infection with the virus is positively associated with onset, development, progression, severity or metastasis of the cancer.

EBV infection is implicated in several cancers, as reviewed e.g. in Jha et al., Front Microbiol. (2016) 7:1602, which is hereby incorporated by reference in its entirety.

In some embodiments, the cancer to be treated/prevented is an EBV-associated cancer. In some embodiments, the cancer is a cancer which is caused or exacerbated by infection with EBV, a cancer for which infection with EBV is a risk factor and/or a cancer for which infection with EBV is positively associated with onset, development, progression, severity or metastasis of the cancer. The cancer may be characterised by EBV infection, e.g. the cancer may comprise cells infected with EBV. Such cancers may be referred to as EBV-positive cancers.

EBV-associated cancers which may be treated/prevented in accordance with the present disclosure include B cell-associated cancers such as Burkitt's lymphoma, post-transplant lymphoproliferative disease (PTLD), central nervous system lymphoma (CNS lymphoma), Hodgkin's lymphoma, non-Hodgkin's lymphoma, and EBV-associated lymphomas associated with immunodeficiency (including e.g. EBV-positive lymphoma associated with X-linked lymphoproliferative disorder, EBV-positive lymphoma associated with HIV infection/AIDS, and oral hairy leukoplakia), and epithelial cell-related cancers such as nasopharyngeal carcinoma (NPC) and gastric carcinoma (GC).

In some embodiments, the cancer is selected from lymphoma (e.g. EBV-positive lymphoma), head and neck squamous cell carcinoma (HNSCC; e.g. EBV-positive HNSCC), nasopharyngeal carcinoma (NPC; e.g. EBV-positive NPC), and gastric carcinoma (GC; e.g. EBV-positive GC).

In some embodiments the cancer is a cancer in which the target antigen for the CAR is pathologically implicated. That is, in some embodiments the cancer is a cancer which is caused or exacerbated by the expression of the target antigen, a cancer for which expression of the target antigen is a risk factor and/or a cancer for which expression of the target antigen is positively associated with onset, development, progression, severity or metastasis of the cancer. The cancer may be characterised by expression of the target antigen, e.g. the cancer may comprise cells expressing the target antigen. Such cancers may be referred to as being positive for the target antigen.

A cancer which is ‘positive’ for the target antigen may be a cancer comprising cells expressing the target antigen (e.g. at the cell surface). A cancer which is ‘positive’ for the target antigen may overexpress the target antigen. Overexpression of the target antigen may be determined by detection of a level of gene or protein expression of the target antigen which is greater than the level of expression by equivalent non-cancerous cells/non-tumor tissue.

In some embodiments the target antigen is a cancer cell antigen as described herein. In some embodiments the target antigen is CD30.

In some embodiments the cancer is a cancer in which CD30 is pathologically implicated. That is, in some embodiments the cancer is a cancer which is caused or exacerbated CD30 expression, a cancer for which expression of CD30 is a risk factor and/or a cancer for which expression of CD30 is positively associated with onset, development, progression, severity or metastasis of the cancer. The cancer may be characterised by CD30 expression, e.g. the cancer may comprise cells expressing CD30. Such cancers may be referred to as CD30-positive cancers.

A CD30-positive cancer may be a cancer comprising cells expressing CD30 (e.g. cells expressing CD30 protein at the cell surface). A CD30-positive cancer may overexpress CD30. Overexpression of CD30 can be determined by detection of a level of gene or protein expression of CD30 which is greater than the level of expression by equivalent non-cancerous cells/non-tumor tissue.

CD30-positive cancers are described e.g. in van der Weyden et al., Blood Cancer Journal (2017) 7:e603 and Muta and Podack, Immunol Res (2013), 57(1-3):151-8, both of which are hereby incorporated by reference in their entirety. CD30 is expressed on small subsets of activated T and B lymphocytes, and by various lymphoid neoplasms including classical Hodgkin's lymphoma and anaplastic large cell lymphoma. Variable expression of CD30 has also been shown for peripheral T cell lymphoma, not otherwise specified (PTCL-NOS), adult T cell leukemia/lymphoma, cutaneous T cell lymphoma (CTCL), extra-nodal NK-T cell lymphoma, various B cell non-Hodgkin's lymphomas (including diffuse large B cell lymphoma, particularly EBV-positive diffuse large B cell lymphoma), and advanced systemic mastocytosis. CD30 expression has also been observed in some non-hematopoietic malignancies, including germ cell tumors and testicular embryonal carcinomas.

The transmembrane glycoprotein CD30, is a member of the tumor necrosis factor receptor superfamily (Falini et al., Blood (1995) 85(1):1-14). Members of the TNF/TNF-receptor (TNF-R) superfamily coordinate the immune response at multiple levels and CD30 plays a role in regulating the function or proliferation of normal lymphoid cells. CD30 was originally described as an antigen recognized by a monoclonal antibody, Ki-1, which was raised by immunizing mice with a HL-derived cell line, L428 (Muta and Podack, Immunol Res (2013) 57:151-158). CD30 antigen expression has been used to identify ALCL and Reed-Stemberg cells in Hodgkin's disease (Falini et al., Blood (1995) 85(1):1-14). With the wide expression in the lymphoma malignant cells, CD30 is therefore a potential target for developing both antibody-based immunotherapy and cellular therapies. Importantly, CD30 is not typically expressed on normal tissues under physiologic conditions, thus is notably absent on resting mature or precursor B or T cells (Younes and Ansell, Semin Hematol (2016) 53:186-189). Brentuximab vedotin, an antibody-drug conjugate that targets CD30 was initially approved for the treatment of CD30-positive HL (Adcetris US Package Insert 2018). Data from brentuximab vedotin trials support CD30 as a therapeutic target for the treatment of CD30-positive lymphoma, although toxicities associated with its use are of concern.

Hodgkin lymphoma (HL) is an uncommon malignancy involving lymph nodes and the lymphatic system. The incidence of HL is bimodal with most patients diagnosed between 15 and 30 years of age, followed by another peak in adults aged 55 years or older. In 2019 it is estimated there will be 8,110 new cases (3,540 in females and 4570 in males) in the United States and 1,000 deaths (410 female and 590 males) from this disease (American Cancer Society 2019). Based on 2012-2016 cases in National Cancer Institute's SEER database, the incidence rate for HL for the pediatric HL patients in US is as follows: Age 1-4: 0.1; Age 5-9: 0.3; Age 10-14: 1.3; Age 15-19: 3.3 per 100,000 (SEER Cancer Statistics Review, 1975-2016]). The World Health Organization (WHO) classification divides HL into 2 main types: classical Hodgkin lymphoma (cHL) and nodular lymphocyte-predominant Hodgkin lymphoma (NLPHL). In Western countries, cHL accounts for 95% and NLPHL accounts for 5% of all HL (National Comprehensive Cancer Network Guidelines 2019).

First-line chemotherapy for cHL patients with advanced disease is associated with cure rates between 70% and 75% (Karantanos et al., Blood Lymphat Cancer (2017) 7:37-52). Salvage chemotherapy followed by Autologous Stem Cell Transplant (ASCT) is commonly used in patients who relapse after primary therapy. Unfortunately, up to 50% of the cHL patients experience disease recurrence after ASCT. The median overall survival of patients who relapse after ASCT is approximately two years (Alinari Blood (2016) 127:287-295). Despite aggressive combination chemotherapy, between 10% and 40% of patients do not achieve a response to salvage chemotherapy and there are no randomized clinical trial data supporting ASCT in non-responders. For patients who do not respond to salvage chemotherapy, relapse after ASCT or who are not candidates for this approach, the prognosis continues to be grave and new treatment approaches are urgently needed (Keudell British Journal of Haematology (2019) 184:105-112).

While a majority of the pediatric population (children, adolescents, and young adults) will be cured with currently available therapy, a small fraction of patients may have refractory or relapsed disease and require novel therapies that have an acceptable safety profile with improved efficacy benefit (Flerlage et al., Blood (2018) 132: 376-384; Kelly, Blood (2015) 126: 2452-2458; McClain and Kamdar, in UpToDate 2019; Moskowitz, ASCO Educational Book (2019) 477-486). HL patients treated with high dose chemotherapy during childhood commonly experience treatment-related long-term sequelae, such as cardiac, pulmonary, gonadal, and endocrine toxicity as well as second malignant neoplasms (Castellino et al., Blood (2011) 117(6): 1806-1816).

In some embodiments, a CD30-positive cancer may be selected from: a solid cancer, a hematological cancer, a hematopoietic malignancy, Hodgkin's lymphoma (HL), anaplastic large cell lymphoma (ALCL), ALK-positive anaplastic T cell lymphoma, ALK-negative anaplastic T cell lymphoma, peripheral T cell lymphoma (e.g. PTCL-NOS), T cell leukemia, T cell lymphoma, cutaneous T cell lymphoma (CTCL), NK-T cell lymphoma (e.g. extra-nodal NK-T cell lymphoma), non-Hodgkin's lymphoma (NHL), B cell non-Hodgkin's lymphoma, diffuse large B cell lymphoma (e.g. diffuse large B cell lymphoma-NOS), primary mediastinal B cell lymphoma, EBV-positive B cell lymphoma, EBV-positive diffuse large B cell lymphoma, advanced systemic mastocytosis, a germ cell tumor and testicular embryonal carcinoma.

In some embodiments, the cancer is selected from: a CD30-positive cancer, an EBV-associated cancer, a hematological cancer, a myeloid hematologic malignancy, a hematopoietic malignancy a lymphoblastic hematologic malignancy, myelodysplastic syndrome, leukemia, T cell leukemia, acute myeloid leukemia, chronic myeloid leukemia, acute lymphoblastic leukemia, lymphoma, Hodgkin's lymphoma, non-Hodgkin's lymphoma, B cell non-Hodgkin's lymphoma, diffuse large B cell lymphoma, primary mediastinal B cell lymphoma, EBV-associated lymphoma, EBV-positive B cell lymphoma, EBV-positive diffuse large B cell lymphoma, EBV-positive lymphoma associated with X-linked lymphoproliferative disorder, EBV-positive lymphoma associated with HIV infection/AIDS, oral hairy leukoplakia, Burkitt's lymphoma, post-transplant lymphoproliferative disease, central nervous system lymphoma, anaplastic large cell lymphoma, T cell lymphoma, ALK-positive anaplastic T cell lymphoma, ALK-negative anaplastic T cell lymphoma, peripheral T cell lymphoma, cutaneous T cell lymphoma, NK-T cell lymphoma, extra-nodal NK-T cell lymphoma, thymoma, multiple myeloma, a solid cancer, epithelial cell cancer, gastric cancer, gastric carcinoma, gastric adenocarcinoma, gastrointestinal adenocarcinoma, liver cancer, hepatocellular carcinoma, cholangiocarcinoma, head and neck cancer, head and neck squamous cell carcinoma, oral cavity cancer, oropharyngeal cancer, oropharyngeal carcinoma, oral cancer, laryngeal cancer, nasopharyngeal carcinoma, oesophageal cancer, colorectal cancer, colorectal carcinoma, colon cancer, colon carcinoma, cervical carcinoma, prostate cancer, lung cancer, non-small cell lung cancer, small cell lung cancer, lung adenocarcinoma, squamous lung cell carcinoma, bladder cancer, urothelial carcinoma, skin cancer, melanoma, advanced melanoma, renal cell cancer, renal cell carcinoma, ovarian cancer, ovarian carcinoma, mesothelioma, breast cancer, brain cancer, glioblastoma, prostate cancer, pancreatic cancer, mastocytosis, advanced systemic mastocytosis, germ cell tumor or testicular embryonal carcinoma.

In some embodiments, the cancer may be a relapsed cancer. As used herein, a “relapsed” cancer refers to a cancer which responded to a treatment (e.g. a first line therapy for the cancer), but which has subsequently re-emerged/progressed, e.g. after a period of remission. For example, a relapsed cancer may be a cancer whose growth/progression was inhibited by a treatment (e.g. a first line therapy for the cancer), and which has subsequently grown/progressed.

In some embodiments, the cancer may be a refractory cancer. As used herein, a “refractory” cancer refers to a cancer which has not responded to a treatment (e.g. a first line therapy for the cancer). For example, a refractory cancer may be a cancer whose growth/progression was not inhibited by a treatment (e.g. a first line therapy for the cancer). In some embodiments a refractory cancer may be a cancer for which a subject receiving treatment for the cancer did not display a partial or complete response to the treatment.

In embodiments where the cancer is anaplastic large cell lymphoma, the cancer may be relapsed or refractory with respect to treatment with chemotherapy, brentuximab vedotin, or crizotinib. In embodiments where the cancer is peripheral T cell lymphoma, the cancer may be relapsed or refractory with respect to treatment with chemotherapy or brentuximab vedotin. In embodiments where the cancer is extranodal NK-T cell lymphoma, the cancer may be relapsed or refractory with respect to treatment with chemotherapy (with or without asparaginase) or brentuximab vedotin. In embodiments where the cancer is diffuse large B cell lymphoma, the cancer may be relapsed or refractory with respect to treatment with chemotherapy (with or without rituximab) or CD19 CAR-T therapy. In embodiments where the cancer is primary mediastinal B cell lymphoma, the cancer may be relapsed or refractory with respect to treatment with chemotherapy, immune checkpoint inhibitor (e.g. PD-1 inhibitor) or CD19 CAR-T therapy.

Treatment of a cancer in accordance with the methods of the present disclosure achieves one or more of the following treatment effects: reduces the number of cancer cells in the subject, reduces the size of a cancerous tumor/lesion in the subject, inhibits (e.g. prevents or slows) growth of cancer cells in the subject, inhibits (e.g. prevents or slows) growth of a cancerous tumor/lesion in the subject, inhibits (e.g. prevents or slows) the development/progression of a cancer (e.g. to a later stage, or metastasis), reduces the severity of symptoms of a cancer in the subject, increases survival of the subject (e.g. progression free survival or overall survival), reduces a correlate of the number or activity of cancer cells in the subject, and/or reduces cancer burden in the subject.

Subjects may be evaluated in accordance with the Revised Criteria for Response Assessment: The Lugano Classification (described e.g. in Cheson et al., J Clin Oncol (2014) 32: 3059-3068, incorporated by reference hereinabove) in order to determine their response to treatment. In some embodiments, treatment of a subject in accordance with the methods of the present disclosure achieves one of the following: complete response, partial response, or stable disease.

In some embodiments, treatment of cancer further comprises chemotherapy and/or radiotherapy.

Chemotherapy and radiotherapy respectively refer to treatment of a cancer with a drug or with ionising radiation (e.g. radiotherapy using X-rays or γ-rays). The drug may be a chemical entity, e.g. small molecule pharmaceutical, antibiotic, DNA intercalator, protein inhibitor (e.g. kinase inhibitor), or a biological agent, e.g. antibody, antibody fragment, aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

Chemotherapy may involve administration of more than one drug. A drug may be administered alone or in combination with other treatments, either simultaneously or sequentially dependent upon the condition to be treated.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment. The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs may be selected from: Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alentuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calquence (Acalabrutinib), Campath (Alemtuzumab), Camptosar (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Eriotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Idusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (lbrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paditaxel Albumin-stabilized Nanoparticle Formulation), Navelbine (Vinorelbine Tartrate), Necitumumab, Nelarabine, Neosar (Cyclophosphamide), Neratinib Maleate, Nertynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincuistine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (kdelalisib), Zykadia (Ceritinib) and Zytiga (Abiraterone Acetate).

EBV-infection is also implicated in the development/progression of a variety of autoimmune diseases, such as multiple sclerosis and systemic lupus erythematosus (SLE; see e.g. Ascherio and Munger Curr Top Microbiol Immunol. (2015); 390(Pt 1):365-85), and EBV antigen EBNA2 has recently been shown to associate with genetic regions implicated as risk factors for the development of SLE, multiple sclerosis, rheumatoid arthritis, inflammatory bowel disease, type 1 diabetes, juvenile idiopathic arthritis and celiac disease (Harley et al., Nat Genet. (2018) 50(5): 699-707).

Accordingly, in some embodiments the disease/condition to be treated/prevented in accordance with the present disclosure is selected from: an autoimmune disease, SLE, multiple sclerosis, rheumatoid arthritis, inflammatory bowel disease, type 1 diabetes, juvenile idiopathic arthritis and celiac disease.

Aspects and embodiments of the present disclosure relate to CAR-expressing virus-specific immune cells comprising one or more CARs specific for more than one, non-identical target antigens. In some embodiments, the virus-specific immune cells comprising a CAR specific for CD30 comprise a CAR specific for an antigen other than CD30. For example, Example 4 herein describes virus-specific immune cells comprising a CD30-specific CAR and a CD19-specific CAR.

In some embodiments the cancer to be treated/prevented in accordance with the present invention is a cancer comprising cells expressing one or more of the non-identical target antigens. In some embodiments the cancer is a cancer expressing both of each of the non-identical target antigens.

Applications Relating to Treatment/Prevention of Alloreactive Immune Responses

The CAR-expressing virus-specific immune cells and compositions of the present disclosure can be used in methods involving allotransplantation, e.g. to treat/prevent a disease/condition in a subject.

The CAR-expressing virus-specific immune cells and compositions of the present disclosure are useful in methods to reduce/prevent alloreactive immune responses (particularly T cell-mediated alloreactive immune responses) and the deleterious consequences thereof.

Alloreactive T cells express CD30. Chan et al., J Immunol (2002) 169(4):1784-91 identify CD30-expressing T cells as a subset of activated T cells (also expressing CD25 and CD45RO) having an important role in CD30 alloimmune responses. CD30 expression and the proliferation of CD30-expressing T cells increases in response to alloantigen. Chen et al., Blood (2012) 120(3):691-6 identifies CD30 expression on CD8+ T cell subsets as a potential biomarker for GVHD, and propose CD30 as a therapeutic target for GVHD.

Virus-specific T cells moreover have a more restricted TCR repertoire than polyclonal activated T cells (ATCs), and are therefore less likely to cause GVHD following administration to allogeneic subjects. This is reflected by the low incidence of GVHD in studies of allogeneic EBV-specific T cells (EBVSTs).

The CAR-expressing virus-specific immune cells and compositions of the present disclosure are particularly useful in methods involving allotransplantation, and also in the processing/production of allotransplants.

In particular, the CAR-expressing virus-specific immune cells and compositions are contemplated for use in the production and administration of “off-the-shelf” materials for use in therapeutic and prophylactic methods comprising administration of allogeneic material.

As explained hereinabove, CAR-expressing virus-specific immune cells of the present disclosure are useful for the treatment/prevention of diseases/conditions by adoptive cell transfer. CAR-expressing virus-specific immune cells of the present disclosure are less susceptible to T cell-mediated alloreactive immune responses of the recipient following adoptive transfer, and thus exhibit enhanced proliferation/survival in the recipient after transfer, and superior therapeutic/prophylactic effects.

The CAR-expressing virus-specific immune cells and compositions of the present disclosure are also useful in methods comprising allotransplantation of allogeneic cells other than the CAR-expressing virus-specific immune cells of the present disclosure. In particular, the CAR-expressing virus-specific immune cells and compositions of the present disclosure are useful for depleting allotransplants (populations of cells, tissues and organs) and subjects of alloreactive immune cells (e.g. alloreactive T cells).

In such methods the CAR-expressing virus-specific immune cells and compositions are useful for conditioning of donor and/or recipient subjects, and/or treatment of the allotransplant to reduce/prevent an alloreactive immune response following allotransplantation.

Cells, tissues and organs to be allotransplanted include e.g. immune cells (e.g. adoptive cell transfer), the heart, lung, kidney, liver, pancreas, intestine, face, cornea, skin, hematopoietic stem cells (bone marrow), blood, hands, leg, penis, bone, uterus, thymus, islets of Langerhans, heart valve and ovary. Populations of cells, tissues or organs to be allotransplanted may be referred to as “allotransplants”.

The disease/condition to be treated/prevented by the allotransplantation can be any disease/condition which would derive therapeutic or prophylactic benefit from the allotransplantation. In some embodiments, the disease/condition to be treated/prevented by allotransplantation may e.g. be a T cell dysfunctional disorder, a cancer, an infectious disease or an autoimmune disease.

A T cell dysfunctional disorder may be a disease/condition in which normal T cell function is impaired causing downregulation of the subject's immune response to pathogenic antigens, e.g. generated by infection by exogenous agents such as microorganisms, bacteria and viruses, or generated by the host in some disease states such as in some forms of cancer (e.g. in the form of tumor-associated antigens).

The T cell dysfunctional disorder may comprise T cell exhaustion or T cell anergy. T cell exhaustion comprises a state in which CD8+ T cells fail to proliferate or exert T cell effector functions such as cytotoxicity and cytokine (e.g. IFNγ) secretion in response to antigen stimulation. Exhausted T cells may also be characterised by sustained expression of one or more markers of T cell exhaustion, e.g. PD-1, CTLA-4, LAG-3, TIM-3. The T cell dysfunctional disorder may manifest as an infection, or inability to mount an effective immune response against an infection. The infection may be chronic, persistent, latent or slow, and may be the result of bacterial, viral, fungal or parasitic infection. As such, treatment may be provided to patients having a bacterial, viral or fungal infection. Examples of bacterial infections include infection with Helicobacter pylori. Examples of viral infections include infection with HIV, hepatitis B or hepatitis C. The T cell dysfunctional disorder may be associated with a cancer, such as tumor immune escape. Many human tumors express tumor-associated antigens recognised by T cells and capable of inducing an immune response.

An infectious disease may be e.g. bacterial, viral, fungal, or parasitic infection. In some embodiments, it may be particularly desirable to treat chronic/persistent infections, e.g. where such infections are associated with T cell dysfunction or T cell exhaustion. It is well established that T cell exhaustion is a state of T cell dysfunction that arises during many chronic infections (including viral, bacterial and parasitic), as well as in cancer (Wherry Nature Immunology Vol. 12, No. 6, p 492-499, June 2011). Examples of bacterial infections that may be treated include infection by Bacillus spp., Bordetella pertussis, Clostridium spp., Corynebacterium spp., Vibrio chloerae, Staphylococcus spp., Streptococcus spp. Escherichia, Klebsiella, Proteus, Yersinia, Erwina, Salmonella, Listeria sp, Helicobacter pylori, mycobacteria (e.g. Mycobacterium tuberculosis) and Pseudomonas aeruginosa. For example, the bacterial infection may be sepsis or tuberculosis. Examples of viral infections that may be treated include infection by influenza virus, measles virus, hepatitis B virus (HBV), hepatitis C virus (HCV), human immunodeficiency virus (HIV), lymphocytic choriomeningitis virus (LCMV), Herpes simplex virus and human papilloma virus (HPV). Examples of fungal infections that may be treated include infection by Alternaria sp, Aspergillus sp, Candida sp and Histoplasma sp. The fungal infection may be fungal sepsis or histoplasmosis. Examples of parasitic infections that may be treated include infection by Plasmodium species (e.g. Plasmodium falciparum, Plasmodium yoeli, Plasmodium ovale, Plasmodium vivax, or Plasmodium chabaudi chabaudi). The parasitic infection may be a disease such as malaria, leishmaniasis and toxoplasmosis.

In some embodiments, the disease/condition is an autoimmune disease. In such embodiments the treatment may be aimed at reducing the number of autoimmune effector cells. In some embodiments the autoimmune disease is selected from: diabetes mellitus type 1, celiac disease, Graves' disease, inflammatory bowel disease, multiple sclerosis, psoriasis, rheumatoid arthritis, and systemic lupus erythematosus.

The CAR-expressing virus-specific immune cells and compositions of the present disclosure are also useful for the treatment/prevention of an alloreactive immune response, and diseases/conditions characterised by an alloreactive immune response.

Diseases and conditions characterised by an alloreactive immune response include diseases/conditions caused or exacerbated by alloreactive immune responses associated with allotransplantation. Such diseases/conditions include graft versus host disease (GVHD) and graft rejection, and are described in detail in Perkey and Maillard Annu Rev Pathol. (2018) 13:219-245, which is hereby incorporated by reference in its entirety.

Graft-versus-host disease (GVHD) can occur following allotransplantation of large numbers of donor immune cells, and involves reactivity of donor-derived immune cells against allogeneic recipient cells/tissues/organs. Graft rejection refers to the destruction of transplanted cells/tissue/organs by a recipient's immune system following transplantation. Where graft rejection is of an allotransplant, it may be referred to as allograft rejection.

The CAR-expressing virus-specific immune cells and compositions of the present disclosure may be used to deplete alloreactive T cells in an allotransplant, which could otherwise lead to graft versus host disease (GVHD) in a recipient upon allotransplantation.

The CAR-expressing virus-specific immune cells and compositions of the present disclosure may be used to deplete alloreactive T cells in a donor for an allotransplant (e.g. prior to harvesting/collecting the allotransplant), which could otherwise lead to GVHD in a recipient upon allotransplantation.

The CAR-expressing virus-specific immune cells and compositions of the present disclosure may be used to deplete alloreactive T cells in the recipient for an allotransplant, which could otherwise cause/promote graft rejection.

The present disclosure provides methods of treating/preventing graft-versus-host disease (GVHD) following allotransplantation, comprising administering a CAR-expressing virus-specific immune cell or composition according to the present disclosure to a donor subject for an allotransplant. The present disclosure also provides methods of treating/preventing graft-versus-host disease (GVHD) following allotransplantation, comprising contacting an allotransplant with a CAR-expressing virus-specific immune cell or composition according to the present disclosure. The aim for such methods is to reduce/remove the ability of alloreactive immune cells in the allograft to mount an alloreactive immune response to cells, tissue and/or organs of the recipient for the allotransplant.

The present disclosure provides methods of treating/preventing graft rejection following allotransplantation, comprising administering a CAR-expressing virus-specific immune cell or composition according to the present disclosure to a recipient subject for an allotransplant. The aim for such methods is to reduce/remove the ability of the receipt subject to mount an alloreactive immune response to the allotransplant. The CAR-expressing virus-specific immune cells are useful to eliminate immune cells in the recipient that would otherwise effect an alloreactive immune response against donor cells, tissue and/or organs.

The present disclosure provides methods comprising depleting an allotransplant of alloreactive immune cells (e.g. alloreactive T cells), comprising contacting an allotransplant (e.g. a population of cells, tissue or an organ to be transplanted) with a CAR-expressing virus-specific immune cell or composition of the present disclosure. The methods may comprise administering a CAR-expressing virus-specific immune cell or composition of the present disclosure to a donor subject for the allotransplant. The aim for such methods is to reduce/remove the ability of alloreactive immune cells in the allograft to mount an alloreactive immune response to cells, tissue and/or organs of the recipient for the allotransplant.

In some embodiments the methods comprise one or more of:
obtaining/collecting a population of cells, tissue or organ from a subject;
contacting a population of cells, tissue or organ with a CAR-expressing virus-specific immune cell or composition according to the present disclosure;
culturing a population of cells, tissue or organ in vro or ex vivo in the presence of a CAR-expressing virus-specific immune cell according to the present disclosure;
harvesting/collecting a population of cells, tissue or organ depleted of alloreactive immune cells; and
transplanting/administering a population of cells, tissue or organ depleted of alloreactive immune cells to a subject.

The present disclosure also provides methods comprising depleting a subject of alloreactive immune cells (e.g. alloreactive T cells), comprising administering a CAR-expressing virus-specific immune cell or composition of the present disclosure to the subject. The subject may be a donor subject for an allotransplant, or may be an intended recipient subject for an allotransplant.

In some embodiments the methods comprise one or more of:
administering a CAR-expressing virus-specific immune cell or composition according to the present disclosure to a subject, in order to deplete alloreactive immune cells in the subject;
obtaining/collecting a population of cells, tissue or organ from a subject to which a CAR-expressing virus-specific immune cell or composition according to the present disclosure has been administered; and
transplanting/administering a population of cells, tissue or organ depleted of alloreactive immune cells to a subject.

In some embodiments the methods comprise one or more of:
administering a CAR-expressing virus-specific immune cell or composition according to the present disclosure to a subject, in order to deplete alloreactive immune cells in the subject; and
transplanting/administering a population of cells, tissue or organ to a subject to which a CAR-expressing virus-specific immune cell or composition according to the present disclosure have previously been administered.

Depletion of alloreactive immune cells may result in e.g. a 2-fold, 10-fold, 100-fold, 1000-fold, 10000-fold or greater reduction in the quantity of alloreactive immune cells in the allotransplant or subject.

The methods may be performed in viro or ex vivo, or in vivo in a subject. Method steps performed in vitro or ex vivo may comprise in vitro or ex vivo cell culture.

The methods may further comprise method steps for the production of CAR-expressing virus-specific immune cells and compositions according to the present disclosure.

In some embodiments, administration of a CAR-expressing virus-specific immune cell or composition according to the present disclosure to a recipient subject for an allotransplantation and allotransplantation are performed simultaneously (i.e. at the same time, or within e.g. 1 hr, 2 hrs, 3 hrs, 4 hrs, 5 hrs, 6 hrs, 8 hrs, 12 hrs, 24 hrs, 36 hrs or 48 hrs).

In some embodiments, administration of a CAR-expressing virus-specific immune cell or composition according to the present disclosure to a recipient subject for an allotransplantation and allotransplantation are performed sequentially. The time interval between administration of a CAR-expressing virus-specific immune cell or composition and allotransplantation may be any time interval, including hours, days, weeks, months, or years. The CAR-expressing virus-specific immune cell or composition may be administered to the recipient subject before or after allotransplantation. The CAR-expressing virus-specific immune cell or composition are preferably administered to the recipient subject prior to allotransplantation.

In some embodiments, administration of a CAR-expressing virus-specific immune cell or composition according to the present disclosure to a donor subject for an allotransplantation and collection of the allotransplant (i.e. collection of the cells, tissue and/or an organ) from the subject are performed simultaneously (i.e. at the same time, or within e.g. 1 hr, 2 hrs, 3 hrs, 4 hrs, 5 hrs, 6 hrs, 8 hrs, 12 hrs, 24 hrs, 36 hrs or 48 hrs). In some embodiments administration of a CAR-expressing virus-specific immune cell or composition according to the present disclosure to a donor subject for an allotransplantation and collection of the allotransplant (i.e. collection of the cells, tissue and/or an organ) from the subject are performed sequentially. The time interval between administration of a CAR-expressing virus-specific immune cell or composition and collection of the allotransplant may be any time interval, including hours, days, weeks, months, or years. The CAR-expressing virus-specific immune cell or composition may be administered to the donor subject before or after collection of the allotransplant. The CAR-expressing virus-specific immune cell or composition are preferably administered to the donor subject prior to collection of the allotransplant.

In some embodiments, the methods comprise additional intervention to treat/prevent an alloreactive immune response, graft rejection and/or GVHD.

In some embodiments, the methods to treat/prevent alloreactivity, graft rejection and/or GVHD comprise administration of immunosuppressive and/or lymphodepletive therapy such as treatment with 40 corticosteroids (e.g. prednisolone, hydrocortisone), calcineurin inhibitors (e.g. cyclosporin, tacrolimus) anti-proliferative agents (e.g. azathioprinem, mycophenolic acid) and/or mTOR inhibitors (e.g. sirolimus, everolimus).

In some embodiments, the methods to treat/prevent alloreactivity and/or graft rejection comprise antibody therapy, such as treatment with monoclonal anti-IL-2Rα receptor antibodies (e.g. basiliximab, daclizumab), anti-T cell antibodies (e.g. anti-thymocyte globulin, anti-lymphocyte globulin) and/or anti-CD20 antibodies (e.g. rituximab).

In some embodiments, the methods to treat/prevent alloreactivity and/or graft rejection comprise blood transfusion and/or bone marrow transplantation.

Where a method is disclosed herein, the present disclosure also provides the CAR-expressing virus-specific immune cells and compositions of the present disclosure for use in such methods. Also provided is the use of the CAR-expressing virus-specific immune cells or compositions of the present disclosure in the manufacture of products (e.g. medicaments) for use in such methods.

In some embodiments, the methods of various aspects of the present disclosure cause less depletion and/or increased survival of non-alloreactive immune cells as compared to methods employing immunosuppressive agent(s). For example, the present methods are useful for preserving/maintaining the non-alloreactive immune cell compartment in a recipient subject for an allotransplant, or in an allotransplant.

In some embodiments of the methods of the present disclosure comprising allotransplantation, the present methods are associated with an increased number/proportion of non-alloreactive immune cells in the recipient subject for the allotransplant as compared to methods involving treatment with an immunosuppressive agent. In some embodiments of the methods of the present disclosure comprising adoptive transfer of allogeneic immune cells, the present methods are associated with an increased number/proportion of non-alloreactive immune cells in the recipient subject for the allogeneic immune cells as compared to methods involving treatment with an immunosuppressive agent.

In some embodiments of the methods of the present disclosure comprising allotransplantation, the present methods are associated with an increased number/proportion of non-alloreactive immune cells in the allotransplant as compared to methods involving treatment with an immunosuppressive agent.

The present disclosure also provides the CAR-expressing virus-specific immune cell or composition of the present disclosure for use in a method of:
killing a cell expressing the target antigen for which the CAR is specific (e.g. a cell expressing CD30);
killing a cell infected with, or presenting a peptide of an antigen of, the virus for which the virus-specific immune cell is specific (e.g. a cell infected with EBV, or presenting a peptide of an EBV antigen); and/or
killing an alloreactive immune cell (e.g. a T cell expressing CD30).

The present disclosure also provides the use of such CAR-expressing virus-specific immune cells and compositions in such methods, and methods using the CAR-expressing virus-specific immune cell and compositions to such ends.

Subjects

The subject in accordance with aspects the present disclosure may be any animal or human. The subject is preferably mammalian, more preferably human. The subject may be a non-human mammal, but is more preferably human. The subject may be male or female. The subject may be a patient. A subject may have been diagnosed with a disease/condition described herein requiring treatment, may be suspected of having such a disease/condition, or may be at risk of developing/contracting such a disease/condition.

In embodiments according to the present disclosure, the subject is preferably a human subject. In some embodiments, the subject to be treated according to a therapeutic or prophylactic method of the present disclosure is a subject having, or at risk of developing, a disease/condition described herein. In embodiments according to the present invention, a subject may be selected for treatment according to the methods based on characterisation for certain markers of such a disease/condition.

A subject may be an allogeneic subject with respect to an intervention in accordance with the present disclosure. A subject to be treated/prevented in accordance with the present disclosure may be genetically non-identical to the subject from which the CAR-expressing virus-specific immune cells are derived. A subject to be treated/prevented in accordance with the present disclosure may be HLA mismatched with respect to the subject from which the CAR-expressing virus-specific immune cells are derived. A subject to be treated/prevented in accordance with the present disclosure may be HLA matched with respect to the subject from which the CAR-expressing virus-specific immune cells are derived.

The subject to which cells are administered in accordance with the present disclosure may be allogeneic/non-autologous with respect to the source from which the cells are/were derived. The subject to which cells are administered may be a different subject to the subject from which cells are/were obtained for the production of the cells to be administered. The subject to which the cells are administered may be genetically non-identical to the subject from which cells are/were obtained for the production of the cells to be administered.

The subject to which cells are administered may comprise MHC/HLA genes encoding MHC/HLA molecules which are non-identical to the MHC/HLA molecules encoded by the MHC/HLA genes of the subject from which cells are/were obtained for the production of the cells to be administered. The subject to which cells are administered may comprise MHC/HLA genes encoding MHC/HLA molecules which are identical to the MHC/HLA molecules encoded by the MHC/HLA genes of the subject from which cells are/were obtained for the production of the cells to be administered.

In some embodiments, the subject to which cells are administered is HLA matched with respect to the subject from which cells are/were obtained for the production of the cells to be administered. In some embodiments, the subject to which cells are administered is a near or complete HLA match with respect to the subject from which cells are/were obtained for the production of the cells to be administered. In some embodiments, the subject is a 24/8 (i.e. 4/8, 5/8, 6/8, 7/8 or 8/8) match across HLA-A, -B, -C, and -DRB1. In some embodiments, the subject is a 25/10 (i.e. 5/10, 6/10, 7/10, 8/10, 9/10 or 10/10) match across HLA-A, -B, -C, -DRB1 and -DQB1. In some embodiments, the subject is a 26/12 (i.e. 6/12, 7/12, 8/12, 9/12, 10/12, 11/12 or 12/12) match across HLA-A, -B, -C, -DRB1, -DQB1 and -DPB1. In some embodiments, the subject is an 8/8 match across HLA-A, -B, -C, and -DRB1. In some embodiments, the subject is a 10/10 match across HLA-A, -B, -C, -DRB1 and -DQB1. In some embodiments, the subject is a 12/12 match across HLA-A, -B, -C, -DRB1, -DQB1 and -DPB1.

Sequence Identity

Pairwise and multiple sequence alignment for the purposes of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

Sequences

| SEQ | | |
|---|---|---|
| ID | | |
| NO: | DESCRIPTION | SEQUENCE |
| 1 | Human CD30 | MRVLLAALGLLFLGALRAFPQDRPFEDTCHGNPSHYYDKAVRRCCYRCPMGLFPTQQCPQRPTD |
| | isoform 1 (UniProt: | CRKQCEPDYYLDEADRCTACVTCSRDDLVEKTPCAWNSSRVCECRPGMFCSTSAVNSCARCFF |
| | P28908-1, v1) | HSVCPAGMIVKFPGTAQKNTVCEPASPGVSPACASPENCKEPSSGTIPQAKPTPVSPATSSASTM |
| | | PVRGGTRLAQEAASKLTRAPDSPSSVGRPSSDPGLSPTQPCPEGSGDCRKQCEPDYYLDEAGR |
| | | CTACVSCSRDDLVEKTPCAWNSSRTCECRPGMICATSATNSCARCVPYPICAAETVTKPQDMAE |
| | | KDTTFEAPPLGTQPDCNPTPENGEAPASTSPTQSLLVDSQASKTLPIPTSAPVALSSTGKPVLDAG |
| | | PVLFWVILVLVVVVGSSAFLLCHRRACRKRIRQKLHLCYPVQTSQPKLELVDSRPRRSSTQLRSGA |
| | | SVTEPVAEERGLMSQPLMETCHSVGAAYLESLPLQDASPAGGPSSPRDLPEPRVSTEHTNNKIEK |
| | | IYIMKADTVIVGTVKAELPEGRGLAGPAEPELEEELEADHTPHYPEQETEPPLGSCSDVMLSVEEE |
| | | GKEDPLPTAASGK |
| 2 | Human CD30 | MSQPLMETCHSVGAAYLESLPLQDASPAGGPSSPRDLPEPRVSTEHTNNKIEKIYIMKADTVIVGT |
| | isoform 2 (UniProt: | VKAELPEGRGLAGPAEPELEEELEADHTPHYPEQETEPPLGSCSDVMLSVEEEGKEDPLPTAAS |
| | P28908-2) | GK |
| 3 | Human CD30 | MFCSTSAVNSCARCFFHSVCPAGMIVKFPGTAQKNTVCEPASPGVSPACASPENCKEPSSGTIP |
| | isoform 3 (UniProt: | QAKPTPVSPATSSASTMPVRGGTRLAQEAASKLTRAPDSPSSVGRPSSDPGLSPTQPCPEGSGD |
| | P28908-3) | CRKQCEPDYYLDEAGRCTACVSCSRDDLVEKTPCAWNSSRTCECRPGMICATSATNSCARCVPY |
| | | PICAAETVTKPQDMAEKDTTFEAPPLGTQPDCNPTPENGEAPASTSPTQSLLVDSQASKTLPIPT |
| | | SAPVALSSTGKPVLDAGPVLFWVILVLVVVVGSSAFLLCHRRACRKRIRQKLHLCYPVQTSQPKLE |
| | | LVDSRPRRSSTLRSGASVTEPVAEERGLMSQPLMETCHSVGAAYLESLPLQDASPAGGPSSPRD |
| | | LPEPRVSTEHTNNKIEKIYIMKADTVIVGTVKAELPEGRGLAGPAEPELEEELEADHTPHYPEQ |
| | | ETEPPLGSCSDVMLSVEEEGKEDPLPTAASGK |
| 4 | Human CD30 signal | MRVLLAALGLLFLGALRA |
| | peptide | |
| 5 | Human CD30 | FPQDRPFEDTCHGNPSHYYDKAVRRCCYRCPMGLFPTQQCPQRPTDCRKQCEPDYYLDEADR |
| | extracellular | CTACVTCSRDDLVEKTPCAWNSSRVCECRPGMFCSTSAVNSCARCFFHSVCPAGMIVKFPGTA |
| | domain | QKNTVCEPASPGVSPACASPENCKEPSSGTIPQAKPTPVSPATSSASTMPVRGGTRLAQEAASK |
| | | LTRAPDSPSSVGRPSSDPGLSPTQPCPEGSGDCRKQCEPDYYLDEAGRCTACVSCSRDDLVEK |
| | | TPCAWNSSRTCECRPGMICATSATNSCARCVPYPICAAETVTKPQDMAEKDTTFEAPPLGTQPD |
| | | CNPTPENGEAPASTSPTQSLLVDSQASKTLPIPTSAPVALSSTGKPVLDAG |
| 6 | Human CD30 | PVLFWVILVLVVVVGSSAFLL |
| | transmembrane | |
| | domain | |
| 7 | Human CD30 | CHRRACRKRIRQKLHLCYPVQTSQPKLELVDSRPRRSSTQLRSGASVTEPVAEERGLMSQPLME |
| | cytoplasmic domain | TCHSVGAAYLESLPLQDASPAGGPSSPRDLPEPRVSTEHTNNKIEKIYIMKADTVIVGTVKAEL |
| | | PEGRGLAGPAEPELEEELEADHTPHYPEQETEPPLGSCSDVMLSVEEEGKEDPLPTAASGK |
| 8 | HRS3 HC-CDR1 | GYIFIIYT |
| 9 | HRS3 HC-CDR2 | INPSSGCS |
| 10 | HRS3 HC-CDR3 | ARRADYGNYEYTWFAY |
| 11 | HRS3 LC-CDR1 | QNVGTN |
| 12 | HRS3 LC-CDR2 | SAS |
| 13 | HRS3 LC-CDR3 | QQYHTYPLT |
| 14 | HRS3 VH | QVQLQQSGAELARPGASVKMSCKASGYTFTTYTIHWVRRRPGHDLEWIGYINPSSGCSDYNQNF |
| | | KGKTTLTADKSSNTAYMQLNSLTSEDSAVYYCARRADYGNYEYTWFAYWGQGTTVTVSS |
| 15 | HRS3 VL | VIELTQSPKFMSTSVGDRVNVTYKASQNVGTNVAWFQQKPGQSPKVLIYSASYRYSGVPDRFTG |
| | | SGSGTDFTLTISNVQSEDLAEYFCQQYHTYPLTFGGGTKLEIK |
| 16 | G₄S | GGGGS |
| 17 | HRS3 scFv linker | SGGGSGGGGSGGGGS |
| 18 | HRS3 scFv | QVQLQQSGAELARPGASVKMSCKASGYTFTTYTIHWVRRRPGHDLEWIGYINPSSGCSDYNQNF |
| | | KGKTTLTADKSSNTAYMQLNSLTSEDSAVYYCARRADYGNYEYTWFAYWGQGTTVTVSSSGGG |
| | | SGGGGSGGGGSVIELTQSPKFMSTSVGDRVNVTYKASQNVGTNVAWFQQKPGQSPKVLIYSAS |
| | | YRYSGVPDRFTGSGSGTDFTLTISNVQSEDLAEYFCQQYHTYPLTFGGGTKLEIK |
| 19 | HRS3 epitope | ATSSASTMPVRGGTRLAQEAASKLTRAPDSPSSVGRPSSDPGLSPTQPCPEGSGDCRKQCEPD |
| | | YYLDEAGRCTACVSCSRDDLVEKTPCAWNSSRTCECRPGMICATSATNSCARCVPYPICAAETV |
| | | TKPQDMAEKDTTFEAPPLGTQPDC |
| 20 | Human CD28 | FWVLVVVGGVLACYSLLVTVAFII |
| | transmembrane | |
| | domain | |
| 21 | Human CD3ζ | LCYLLDGILFIYGVILTALFL |
| | transmembrane | |
| | domain | |
| 22 | Human CD8α | IYWAPLAGTCGVLLLSLVITLYCNHRN |
| 23 | transmembrane | YXXL/I |
| 24 | domain | Wherein X = any amino acid |
| | ITAM consensus | YXXL/I(X)₆₋₈YXXL/I |
| | Larger ITAM | Wherein X = any amino acid |
| | consensus | |
| 25 | Human CD3ζ | RVKFSRSADAPAYQQGQNQLYNELNLGRREEYDVLDKRRGRDPEMGGKPRRKNPQEGLYNEL |
| | intracellular | QKDKMAEAYSEIGMKGERRRGKGHDGLYQGLSTATKDTYDALHMQALPPR |
| | domain | |
| 26 | Human CD28 | FWVRSKRSRLLHSDYMNMTPRRPGPTRKHYQPYAPPRDFAAYRS |
| | intracellular | |
| | domain | |
| 27 | Human CD28 | FWVRSKRSRLLHSDYMNMTPRRPGPTRKHYQAYAAARDFAAYRS |
| 28 | intracellular | FWVRSKRSRLLHSDYMNMTPRRPGPTRKHYQPYAPPRDFAAYRSRVKFSRSADAPAYQQGQN) |
| | domain | QLYNELNLGRREEYDVLDKRRGRDPEMGGKPRRKNPQEGLYNELQKDKMAEAYSEIGMKGERR |
| | With mutated Ick | RGKGHDGLYQGLSTATKDTYDALHMQALPPR |
| | binding site | |
| | CAR signalling | |
| | domain 1 | |
| 29 | Human IgG1 CH1- | EPKSCDKTHTCP |
| | CH2 hinge region | |
| 30 | Human IgG1 CH1- | EPKSPDKTHTCP |
| | CH2 hinge region | |
| | C103P variant | |
| 31 | Human IgG1 CH2- | PCPAPELLGGPSVFLFPPKPKDTLMISRTPEVTCVVVDVSHEDPEVKFNWYVDGVEVHNAKTKPR |
| | CH3 hinge region | EEQYNSTYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRWQQGN |
| | | VFSCSVMHEALHNHYTOKSLSLSPGK |
| 32 | Human IgG1 CH2- | PCPAPPVAGPSVFLFPPKPKDTLMIARTPEVTCVVVDVSHEDPEVKFNWYVDGVEVHNAKTKPRE |
| | CH3 hinge region | EQYNSTYRVVSVLTVLHQDWLNGKEYKCKVSNKALPAPIEKTISKAKGQPREPQVYTLPPSRDEL |
| | variant | TKNQVSLTCLVKGFYPSDIAVEWESNGQPENNYKTTPPVLDSDGSFFLYSKLTVDKSRWQQGNV |
| | | FSCSVMHEALHNHYTQKSLSLSPGKKDPK |
| 33 | CAR hinge region | EPKSPDKTHTCPPCPAPELLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKL |
| | | TVDKSRWQQGNVFSCSVMHEALHNHYTQKSLSLSPGK |
| 34 | Signal peptide 1 | MDFQVQIFSFLLISASVIMS |
| 35 | CD30.CAR (lacking | QVQLQQSGAELARPGASVKMSCKASGYTFTTYTIHWVRRRPGHDLEWIGYINPSSGCSDYNQNF |
| | signal peptide) | KGKTTLTADKSSNTAYMQLNSLTSEDSAVYYCARRADYGNYEYTWFAYWGQGTTVTVSSSGGG |
| | | SGGGGSGGGGSVIELTQSPKFMSTSVGDRVNVTYKASQNVGTNVAWFQQKPGQSPKVLIYSAS |
| | | YRYSGVPDRFTGSGSGTDFTLTISNVQSEDLAEYFCQQYHTYPLTFGGGTKLEIKRSDPAEPKSP |
| | | DKTHTCPPCPAPELLGGPSVFLFPPKPKDTLMISRTPEVTCVWDVSHEDPEVKFNWYVDGVEVH |
| | | NAKTKPREEQYNSTYRVVSVLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPVLDSDGSFFLYSKLTVDKS |
| | | RWQQGNVFSCSVMHEALHNHYTQKSLSLSPGKKDPKFWVLVWGGVLACYSLLVTVAFIIFWVR |
| | | SKRSRLLHSDYMNMTPRRPGPTRKHYQPYAPPRDFAAYRSRVKFSRSADAPAYQQGQNQLYNE |
| | | LNLGRREEYDVLDKRRGRDPEMGGKPRRKNPQEGLYNELQKDKMAEAYSEIGMKGERRRGKG |
| | | HDGLYQGLSTATKDTYDALHMQALPPR |
| 36 | CD30.CAR (With | MDFQVQIFSFLLISASVIMSRMAQVQLQQSGAELARPGASVKMSCKASGYTFTTYTIHWVRRRPG |
| | signal peptide) | HDLEWIGYINPSSGCSDYNQNFKGKTTLTADKSSNTAYMQLNSLTSEDSAVYYCARRADYGNYE |
| | | YTWFAYWGQGTTVTVSSSGGGSGGGGSGGGGSVIELTQSPKFMSTSVGDRVNVTYKASQNVG |
| | | TNVAWFQQKPGQSPKVLIYSASYRYSGVPDRFTGSGSGTDFTLTISNVQSEDLAEYFCQQYHTYP |
| | | LTFGGGTKLEIKRSDPAEPKSPDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDWLNGKEYKCKVSNKA |
| | | LPAPIEKTISKAKGQPREPQVYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQKSLSLSPGKKDPKFWVLVV |
| | | VGGVLACYSLLVTVAFIIFWVRSKRSRLLHSDYMNMTPRRPGPTRKHYQPYAPPRDFAAYRSRVK |
| | | FSRSADAPAYQQGQNQLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPR |
| 37 | FMC63 HC-CDR1 | GVSLPDYG |
| 38 | FMC63 HC-CDR2 | IWGSETT |
| 39 | FMC63 HC-CDR3 | AKHYYYGGSYAMDY |
| 40 | FMC63 LC-CDR1 | QDISKY |
| 41 | FMC63 LC-CDR2 | HTS |
| 42 | FMC63 LC-CDR3 | QQGNTLPYT |
| 43 | FMC63 VH | EVKLQESGPGLVAPSQSLSVTCTVSGVSLPDYGVSWIRQPPRKGLEWLGVIWGSETTYYNSALK |
| | | SRLTIIKDNSKSQVFLKMNSLQTDDTAIYYCAKHYYYGGSYAMDYWGQGTSVTVSS |
| 44 | FMC63VL | DIQMTQTTSSLSASLGDRVTISCRASQDISKYLNWYQQKPDGTVKLLIYHTSRLHSGVPSRFSGSG |
| | | SGTDYSLTISNLEQEDIATYFCQQGNTLPYTFGGGTKLEIT |
| 45 | (G₄S)₃ linker | GGGGSGGGGSGGGGS |
| 46 | FMC63 SCFv | MALPVTALLLPLALLLHAARPDIQMTQTTSSLSASLGDRVTISCRASQDISKYLNWYQQKPDGTVK |
| | | LLIYHTSRLHSGVPSRFSGSGSGTDYSLTISNLEQEDIATYFCQQGNTLPYTFGGGTKLEITGGGG |
| | | SGGGGSGGGGSEVKLQESGPGLVAPSQSLSVTCTVSGVSLPDYGVSWIRQPPRKGLEWLGVIW |
| | | GSETTYYNSALKSRLTIIKDNSKSQVFLKMNSLQTDDTAIYYCAKHYYYGGSYAMDYWGQGTSVT |
| | | VSS |
| 47 | IgG Hinge 3 | ESKYGPPCPPCPDPK |
| 48 | Human CD28 | FWVVLVVVGGVLACYSLLVTVAFIIFWVRS |
| | transmembrane | |
| | domain v2 | |
| 49 | Human 4-1BB | KRGRKKLLYIFKQPFMRPVQTTQEEDGCSCRFPEEEEGGCEL |
| 50 | intracellular | KRGRKKLLYIFKQPFMRPVQTTQEEDGCSCRFPEEEEGGCELRVKFSRSADAPAYQQGQNQLY |
| | domain | NELNLGRREEYDVLDKRRGRDPEMGGKPRRKNPQEGLYNELQKDKMAEAYSEIGMKGERRRGK |
| | v2 | GHDGLYQGLSTATKDTYDALHMQALPPR |
| | CAR signalling | |
| | domain 2 | |
| 51 | Signal peptide 2 | MALPVTALLLPLALLLHAARP |
| 52 | CD19.CAR (lacking | DIQMTQTTSSLSASLGDRVTISCRASQDISKYLNWYQQKPDGTVKLLIYHTSRLHSGVPSRFSGSG |
| | signal peptide) | SGTDYSLTISNLEQEDIATYFCQQGNTLPYTFGGGTKLEITGGGGSGGGGSGGGGSEVKLQESG |
| | | PGLVAPSQSLSVTCTVSGVSLPDYGVSWIRQPPRKGLEWLGVIWGSETTYYNSALKSRLTIIKDNS |
| | | KSQVFLKMNSLQTDDTAIYYCAKHYYYGGSYAMDYWGQGTSVTVSSESKYGPPCPPCPDPKFW |
| | | VLVVVGGVLACYSLLVTVAFIIFVWRSKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRRKNPQEGLY |
| | | NELQKDKMAEAYSEIGMKGERRRGKGHDGLYQGLSTATKDTYDALHMQALPPR |
| 53 | CD19.CAR (With | MALPVTALLLPLALLLHAARPDIQMTQTTSSLSASLGDRVTISCRASQDISKYLNWYQQKPDGTVK |
| | signal peptide) | LLIYHTSRLHSGVPSRFSGSGSGTDYSLTISNLEQEDIATYFCQQGNTLPYTFGGGTKLEITGGGG |
| | | SGGGGSGGGGSEVKLQESGPGLVAPSQSLSVTCTVSGVSLPDYGVSWIRQPPRKGLEWLGVIW |
| | | GSETTYYNSALKSRLTIIKDNSKSQVFLKMNSLQTDDTAIYYCAKHYYYGGSYAMDYWGQGTSVT |
| | | VSSESKYGPPCPPCPDPKFWVLVVVGGVLACYSLLVTVAFIIFWVRSKRGRKKLLYIFKQPFMRPV |
| | | QTTQEEDGCSCRFPEEEEGGCELRVKFSRSADAPAYQQGQNQLYNELNLGRREEYDVLDKRRG |
| | | RDPEMGGKPRRKNPQEGLYNELQKDKMAEAYSEIGMKGERRRGKGHDGLYQGLSTATKDTYDA |
| | | LHMQALPPR |

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Where a nucleic acid sequence is disclosed herein, the reverse complement thereof is also expressly contemplated.

Methods described herein may be performed in vitro or in vivo. In some embodiments, methods described herein are performed in viro. The term “in vitro” is intended to encompass experiments with cells in culture whereas the term “in vivo” is intended to encompass experiments with intact multi-cellular organisms.

### BRIEF DESCRIPTION OF THE FIGURES

Embodiments and experiments illustrating the principles of the invention will now be discussed with reference to the accompanying figures.

FIG. 1. Scatterplots showing expression of HLA-A2 and CD3 by cells obtained following 7 days culture of: non-transduced EBVSTs derived from a HLA-A2-positive subject (upper left pane) or CD30-CAR construct-transduced EBVSTs derived from a HLA-A2-positive subject (upper right panel); or following 7 days co-culture of alloreactive T cells derived from a HLA-A2-negative subject and non-transduced EBVSTs derived from a HLA-A2-positive subject (bottom left pane), or CD30-CAR construct-transduced EBVSTs derived from a HLA-A2-positive subject (bottom right pane).

FIGS. 2A and 2B. Bar charts showing the cell counts for (2A) EBVSTs (i.e. CD3+, HLA-A2-positive) and (2B) alloreactive T cells (i.e. CD3+, HLA-A2-negative) cells after 7 days.

FIG. 3. Scatterplots showing expression of HLA-A2 and CD71 in cells obtained following 7 days in coculture comprising HLA-A2-positive PBMCs and non-transduced (NT; upper left panel), CD30-CAR construct-transduced (CD30.CAR; upper right panel), CD19-CAR construct-transduced (CD19.CAR: lower left panel), or CD30-CAR and CD19-CAR construct-transduced (CD30+CD19.CAR; lower right panel) EBVSTs derived from a HLA-A2-negative subject.

FIG. 4. Graph showing proliferation of CD30.CAR EBVSTs prepared from blood samples taken from 4 representative donors. The graph shows cumulative fold expansion of the cells grown in culture.

FIGS. 5A and 5B. Graphs showing cytotoxicity of CD30.CAR EBVSTs to (5A) CD30-negative BJAB Burkitt Lymphoma cells and (5B) CD30-positive HDLM2 Hodgkin Lymphoma cells, as determined by ⁵¹Cr release assay, following co-culture of CD30.CAR EBVSTs (effector) and ⁵¹Cr-labelled target cells (target) at the indicated ratios.

FIGS. 6A and 6B. Graphs showing reactivity of CD30.CAR EBVSTs prepared from blood samples taken from 4 representative donors to EBV antigens, as determined by ELISpot analysis. Cells were stimulated with peptides of EBV latent antigens (Latent), peptides of EBV lytic antigens (Lytic) or were not stimulated with antigens (Negative), and the number of spot-forming units per 5×10⁴ cells was determined. (6A) shows the reactivity of EBVSTs not transduced with retrovirus encoding CD30.CAR. (6B) shows the reactivity of CD30.CAR EBVSTs (transduced with retrovirus encoding CD30.CAR).

FIG. 7. Representative images showing the results of PET scans of patient #1 performed prior to infusion of CD30.CAR EBVSTs, and 6 weeks post-infusion.

FIG. 8. Representative images showing the results of PET and CT scans of patient #2 performed prior to infusion of CD30.CAR EBVSTs, and 6 weeks post-infusion.

FIG. 9. Table showing vector copy number in peripheral blood cells as determined by qRT-PCR, within blood samples obtained prior to infusion of CD30.CAR EBVSTs (pre), and at the indicated period post-infusion.

FIG. 10. Bar chart showing the results of analysis of specificity of cells for different antigens within the peripheral blood of patient #1, prior to lymphodepletion (pre-LD), and at the indicated period post-infusion of CD30.CAR EBVSTs, as determined by ELISpot analysis. PBMCs isolated from blood samples at the indicated time points were stimulated with peptides of EBV latent antigens (Latent), peptides of EBV lytic antigens (Lytic), peptides of antigens for other viruses (Other Viruses), peptides of tumor-associated antigens (TAA) antigens, or were not stimulated antigens (No pepmix), and the number of spot-forming units per 3×10⁵ cells was determined.

### EXAMPLES

In the following Examples, the inventors describe the generation of CD30.CAR-expressing EBVSTs their effector activity against cancer cells and their resistance to allorejection.

### Example 1: Generation of Retroviruses Encoding CAR Constructs

| | | Amino acid |
|---|---|---|
| | | sequence of |
| Construct name | CAR domains | encoded CAR |
| [1] CD30-CAR | HRS3 scFv/hIgG1 | SEQ ID NO: 35 |
| | hinge/hIgG1 Fc/CD28 | |
| | TMD/CD28 ICD/CD3ζ ICD | |
| [2] CD19-CAR | FMC63 scFv/IgG | SEQ ID NO: 52 |
| | hinge/CD28 TMD v2/4- | |
| | 1BB ICD/CD3ζ ICD | |

Retrovirus encoding the CD30.CAR construct was prepared by cloning cDNA encoding the CAR into the pSFG-TGFbDNRII retroviral backbone (ATUM, Newark, Calif.).

The plasmid carrying the CD30.CAR sequence, pSFG_CD30CAR, was transfected into HEK 293 Vec-RD114 cells using polyethylenimine (PEI). Cell culture supernatant from the transfected cells was then used to transduce HEK 293Vec-Galv cells (BioVec Pharma, Quebec, Canada) at a density of 5×10⁵ cells/well of a 6-well plate.

The 293Vec-Galv_CD30-CAR cells were trypsinized, and the cells were resuspended in a 15 ml tube at a concentration of 2×100 cells/ml. Two series of dilutions were made, and 1.65 ml of the final cell suspension was diluted and mixed with 220 ml of DMEM+10% FCS. Two hundred μl of this suspension was transferred to wells of a 96-well plate, resulting in 30 cells per plate. The best performing clone was then selected and used to generate retrovirus-containing supernatant. The retrovirus-containing supernatant was subsequently collected, filtered and stored at −80° C. until use.

Retrovirus encoding the CD19.CAR construct was produced by cloning DNA encoding the CD19.CAR was cloned into the pSFG retroviral backbone. The plasmid carrying the CD19.CAR sequence, 85bCD19C, was used to transfect HEK 293 Vec-RD114 cells using polyethylenimine (PEI). The retrovirus-containing the supernatant was subsequently collected, filtered and stored at −80° C. until use.

### Example 2: Generation of CAR-Expressing EBV-Specific T Cells

Peripheral blood mononuclear cells (PBMCs) were isolated from blood samples obtained from healthy donors or lymphoma patients according to the standard Ficoll-Paque density gradient centrifugation method.

Generation of ATCs

Anti-CD3 (clone OKT3) and anti-CD28 agonist antibodies were coated onto wells of tissue culture plates by addition of 0.5 ml of 1:1000 dilution of 1 mg/ml antibodies, and incubation for 2-4 hr at 37° C., or at 4° C. overnight. 1×10⁸ PBMCs (in 2 ml of medium per well) were stimulated by culture on the anti-CD3/CD28 agonist antibody-coated plates in cell culture medium (containing 44.5% Advanced RPMI medium, 44.5% Click's medium, 10% FBS and 1% GlutaMax). The cells were maintained at 37° C. in a 5% CO₂ atmosphere. The next day, 1 ml of the cell culture medium was replaced with fresh cell culture medium containing 20 ng/ml IL-7 and 20 ng/ml IL-15. To maintain ATCs in culture, every 2-4 days, cell culture medium and cytokines were replenished as needed or ATCs were harvested and re-plated in fresh cell culture medium with cytokines. ATCs were harvested and used in experiments for re-stimulation with EBVSTs between days 7-10.

Universal LCLs

LCLs lacking surface expression of HLA class I and HLA class II (i.e. HLA-negative LCLs) were obtained by targeted knockout of genes encoding HLA class I and HLA class II molecules in cells of a lymphoblastoid cell line prepared by EBV-transformation of B cells. The HLA-negative cells were further modified to knockout genes necessary for EBV replication. The resulting cells obtained by the methods are referred to herein as universal LCLs (uLCLs).

Expansion and Transduction of EBV-Specific T Cells (EBVSTs)

PBMCs from a healthy donor were depleted of CD45RA-expressing cells by magnetic cell separation using CD45RA MACS microbeads (Miltenyi Biotec). EBV-specific T cells were expanded by stimulating 2×10⁶ CD45RA-depleted PBMCs (in 2 ml of medium per well) with EBNA1 pepmix (JPT Cat. No. PM-EBV-EBNA1), LMP1 pepmix (JPT Cat. No. PM-EBV-LMP1) and LMP2 pepmix (JPT Cat. No. PM-EBV-LMP2) obtained from JPT Technologies (overlapping 15mer amino acid peptide libraries overlapping by 11 amino acids, spanning the full amino acid sequence of the relevant antigen), in cell culture medium containing 44.5-47% Advanced RPMI, 44.5-47% Click's medium, 10% FBS or 5% growth factor-rich additive and 1% GlutaMax, supplemented with IL-7 (10 ng/ml) and IL-15 (10 ng/m). EBVSTs were maintained at 37° C. in a 5% CO₂ atmosphere.

After 4-6 days, EBVSTs were transduced with CAR-encoding retroviruses described in Example 1 as follows.

Retrovirus-containing supernatants (0.5-1 ml per well) were added to non-tissue culture treated 24-well plates pre-coated with RetroNectin (Takara). After centrifugation of the plate at 2000×g for 60-90 min, retroviral supernatants were removed, and the cells were re-plated at 0.25-0.5×10⁸ cells per well.

After 8-10 days of culture, cells were re-stimulated by co-culture with irradiated, peptide-pulsed autologous activated T cells (ATCs) in the presence of uLCLs. Briefly, 2×10⁸ ATCs were incubated with pepmixes (10 ng pepmix mixture per 1×10⁶ ATCs) at 37° C. for 30 min in CTL medium, and subsequently irradiated at 30Gy and harvested. The peptide-pulsed ATCs were then mixed with the cells in culture and uLCLs (irradiated at 100Gy), in CTL medium containing IL-7 (10 ng/ml) and IL-15 (100 ng/ml), at a ratio of responder cells:peptide-pulsed ATCs:irradiated uLCLs of 1:1:5. Specifically, 1×10⁵ responder cells, 1×10⁵ peptide-pulsed ATCs and 0.5×10⁸ irradiated uLCLs were cultured in 2 mL CTL medium in wells of a 24 well tissue culture plate.

To maintain EBVSTs in culture, every 2-4 days, cell culture medium and cytokines were replenished as needed or EBVSTs were harvested and re-plated in fresh cell culture medium with cytokines. EBVSTs were harvested and used in mixed lymphocyte reactions (MLR) assays between days 15-20.

### Example 3: Evaluation of the Ability of CD30-Specific CAR to Eliminate Alloreactive T Cells and Protect Allogeneic VSTs from Rejection

The inventors investigated the effect of CD30.CAR expression on the ability of VSTs to resist allorejection in vitro.

Generation of Primed Alloreactive T Cells

1-2×10⁸ PBMCs (per well) from the same healthy donor used to generate the EBVSTs were irradiated at 30 Gray and co-cultured with 1×10⁸ PBMCs (per well) from a mismatched donor (with different expression of HLA-A2), in cell culture medium containing 44.5% Advanced RPMI, 44.5% Click's medium, 10% serum, and 1% GlutaMax, supplemented with IL-7 (10 ng/ml) and IL-15 (10 ng/ml). Primed Alloreactive T cells expanded from the PBMCs of the mismatched donor were re-stimulated by plating 0.5×10⁸ cells (in 2 ml of cell culture medium) on anti-CD3/CD28 agonist antibody-coated plates on day 6-10. To maintain alloreactive T cells in culture, every 2-4 days, cell culture medium and cytokines were replenished as needed, or alloreactive T cells were harvested and re-plated in fresh cell culture medium with cytokines. Alloreactive T cells were harvested and used in mixed lymphocyte reaction (MLR) assays with EBVSTs between days 13-17.

To assess allorejection in vitro 0.2×10⁴ PBMCs alloreactive T cells from a HLA-A2-negative subject were co-cultured in a mixed lymphocyte reaction (MLR) assay with:
(i) 0.2×10⁴ EBVSTs generated from the PBMCs of the HLA-A2-positive subject that was used to prime the alloreactive T cells, or
(ii) 0.2×10⁴ EBVSTs generated from the PBMCs of the HLA-A2-positive subject that was used to prime the alloreactive T cells, additionally transduced with construct encoding the CD30-specific CAR.

Human IL-7 (10 ng/ml) and IL-15 (10 ng/ml) were added to the MLR assay.

Flow cytometric analysis was performed after 7 days, and absolute cell numbers were determined using counting beads. T cells derived from the different subjects could be identified in the population obtained following co-culture based on expression of HLA-A2. The Gallios Flow Cytometer (Beckman Coulter) was used to acquire events, and Kaluza Analysis Software (Beckman Coulter) was used for data analysis and graphical representation.

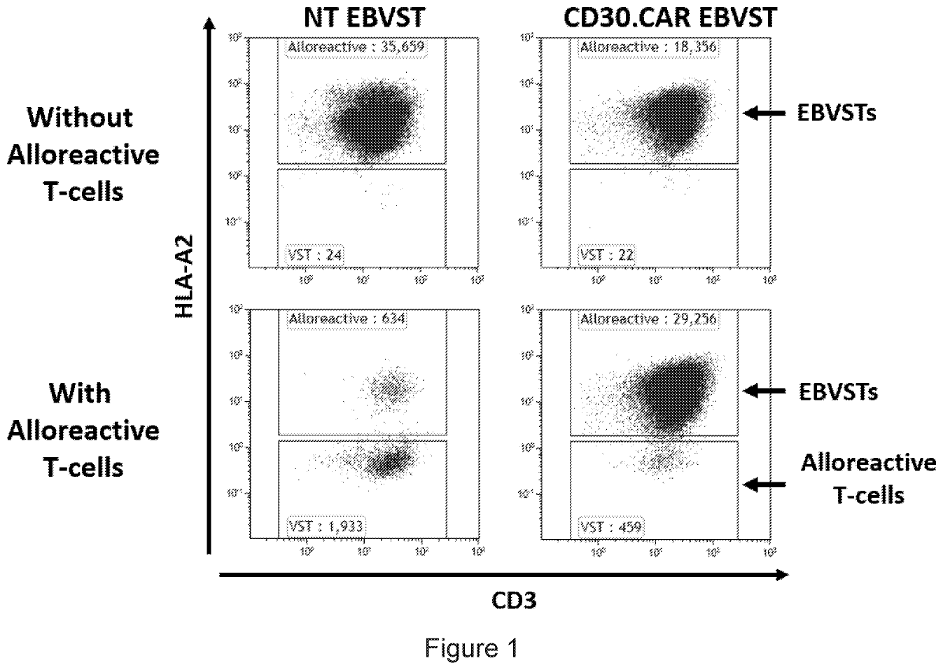

As shown in FIG. 1, the number of non-transduced (NT) EBVSTs derived from the HLA-A2-positive subject was greatly reduced after 7 days co-culture with alloreactive T cells derived from the HLA-A2-negative subject (lower left panel), as compared to when they were cultured in the absence of alloreactive T cells (upper left panel). By contrast, the number of CD30.CAR EBVSTs was increased after 7 days co-culture with alloreactive T cells (lower right panel), as compared to when they were cultured in the absence of alloreactive T cells (upper right panel).

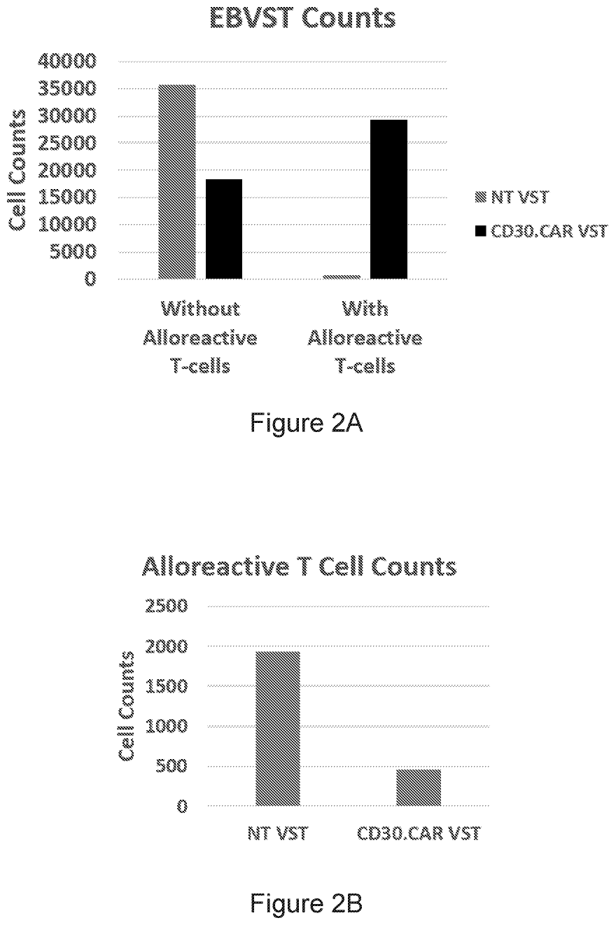

FIG. 2 shows quantification of the flow cytometry data. The non-transduced EBVSTs (NT) were mostly eliminated in the presence of alloreactive T cells, whereas CD30.CAR-expressing EBVSTs were resistant to elimination by alloreactive T cells (FIG. 2A). In addition, quantification of the alloreactive T cell population (CD3+, HLA-A2-negative) revealed that CD30.CAR EBVSTs reduced the number of alloreactive T cells relative to the non-transduced EBVST condition (FIG. 2B).

Thus EBVSTs expressing CD30.CAR were shown to have the ability to reduce the number of alloreactive T cells, and to be protected against allorejection.

### Example 4: Characterisation of EBV-Specific T Cells Expressing a CD19-Specific CAR and a CD30-Specific CAR

The inventors produced and characterised virus-specific T cells engineered to express both a CD19.CAR and a CD30.CAR, and examined whether they could eliminate alloreactive T cells in a mixed lymphocyte reaction.

Briefly, a population of 1×10⁵ PBMCs from a HLA-A2-positive subject depleted of CD19 and CD56 expressing cells was co-cultured in a mixed lymphocyte reaction (MLR) assay with:
(i) 0.1×10⁵ EBVSTs generated from PBMCs of a HLA-A2-negative subject, or
(ii) 0.1×10⁵ EBVSTs generated from PBMCs of a HLA-A2-negative subject, additionally transduced with construct encoding (a) the CD30.CAR, (b) the CD19.CAR, or (c) both the CD30.CAR and the CD19.CAR (CD30+CD19.CAR).

Human IL-2 was added to the MLR assay at 20 IU/ml.

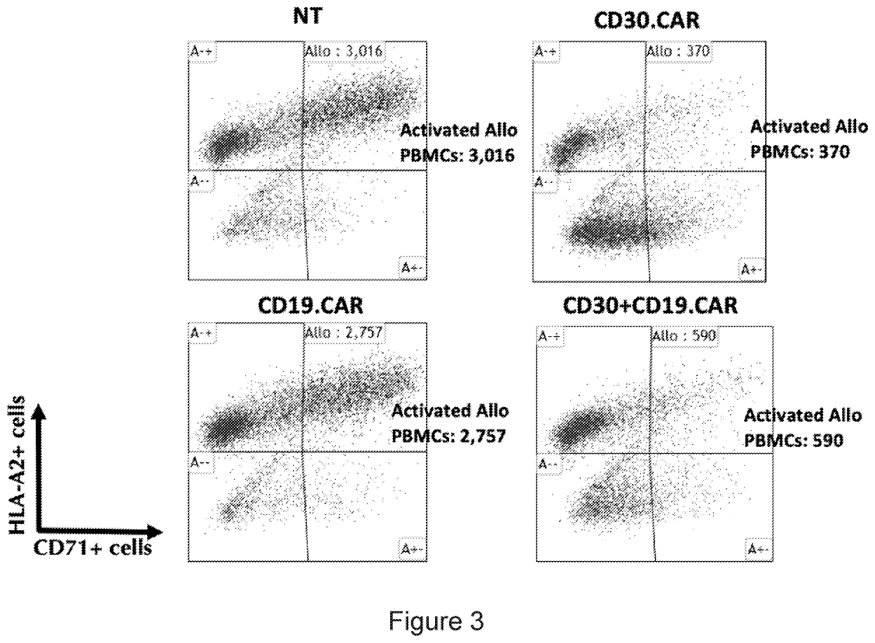

As shown in FIG. 3, both the CD30.CAR EBVSTs (upper right panel) and the CD30+CD19.CAR EBVSTs (lower right panel) greatly reduced the proportion of (and thus avoided rejection by) HLA-A2+ alloreactive T cells (distinguished by the activation marker CD71) by day 7, as compared to non-transduced (NT) EBVSTs (upper left panel) and CD19.CAR EBVSTs (lower left panel).

The inventors thus provide a novel approach to generating an “off-the-shelf” CAR T cell specific for a given target antigen using EBVSTs transduced with both a CAR specific for the target antigen (CD19 in the present example) and a CD30-specific CAR. The ability of such dual CAR-EBVSTs to eliminate alloreactive T cells in vitro, suggests they may be able to avoid rejection and persist long-term in allogeneic recipients in vivo.

### Example 5: Treatment of Cancer Using CD30.CAR EBVSTs

### 5.1 Production and Characterisation of CD30.CAR EBVSTs Produced from Health Donor Subjects

CD30.CAR EBVSTs were manufactured in a GMP facility. Approximately 250 to 400 mL of blood was collected from seven healthy, blood-bank approved donors after obtaining informed consent and in accordance with the guidelines established by the Declaration of Helsinki.

Peripheral blood mononuclear cells (PBMCs) were isolated from blood by density gradient centrifugation. PBMCs were depleted of CD45RA-expressing cells by magnetic cell separation using a clinical grade anti-CD45RA antibody conjugated to magnetic beads, and using and Miltenyi depletion columns (Miltenyi Biotec, Bergisch Gladbach, Germany).

1.5-2.5×10⁷ PBMCs depleted of CD45RA-positive cells were seeded in 30 ml culture medium containing 47.5% Advanced RPMI, 47.5% Click's (EHAA) medium (Irvine Scientific), 2 mM L-glutamine (Thermo Fisher Scientific) and 5% Human Platelet Lysate (HPL; Sexton Biotechnologies), supplemented with IL-7 (10 ng/ml) and IL-15 (10 ng/ml) in G-Rex10 vessels, and activated by stimulation with overlapping peptide libraries (pepmixes) comprising 15mer amino acids overlapping by 11 amino acids, and spanning the entire protein sequences of the relevant antigens. Pepmixes corresponding to EBNA1, LMP1, LMP2, BARF1, BZLF1, BRLF1, BMLF1, BMRF1, BMRF2, BALF2, BNLF2a and BNLF2b were obtained from JPT Technologies (Berlin, Germany). Stimulations employed 5 ng of pepmix for each antigen per 1×10⁶ cells to be stimulated (i.e. for stimulations performed using 2×10⁷ PBMCs depleted of CD45RA-positive cells, 100 ng of each pepmix was used). Stimulation cultures were maintained at 37° C. in a 5% CO₂ atmosphere.

After 4-6 days, EBVSTs produced by the stimulation cultures described in the preceding paragraph were transduced with the CAR-encoding retroviruses described in Example 1, as follows. 2 ml of retrovirus-containing supernatant was mixed with 150 μg Vectofusin-1 in a volume of 2 ml, giving a final volume of 4 ml, and incubated at room temperature for 5-30 min. The retrovirus:Vectofusin-1 mixture was then added to 7-10×10⁸ cells in 8.5 ml culture medium (described in the preceding paragraph), in T75 vessels. Cultures were maintained at 37° C. in a 5% CO₂ atmosphere.

Between days 8 and 10 of culture, 1-2×10⁷ CD30.CAR EBVSTs CD30.CAR EBVSTs produced by transduction as described in the preceding paragraph were transferred to G-Rex100 vessels, and re-stimulated by co-culture with irradiated (at 100 gray) uLCLs (described in Example 2), at a ratio of CD30.CAR EBVSTs to irradiated uLCLs ranging from 1:2 to 1:5 (typically around 1:3). ULCLs express EBV antigens and CD30, as well as other costimulatory molecules, and therefore provide CD30.CAR EBVSTs with antigen stimulation and costimulation, inducing robust proliferation of CD30.CAR EBVSTs without loss of EBV specificity.

Re-stimulation cultures were established in 200 ml culture medium (described in paragraph 3 of section 5.1), and additional culture medium was added as required. 7 to 12 days later, CD30.CAR EBVSTs were harvested and cryopreserved for subsequent infusion.

CD30.CAR EBVSTs prepared from 4 representative healthy donor subjects were evaluated for their ability to proliferate in vitro, cytotoxicity against CD30-expressing and CD30-negative cancer cell lines in vitro, and in order to determine specificity for different EBV antigens.

Analysis of CD30.CAR EBVST Proliferation

Proliferation of CD30.CAR EBVSTs was determined by counting the number of cells using a hemocytometer at various time points during culture (Days 0, 6, 10, 17 18 and 19) during culture, and cumulative fold expansion was calculated.

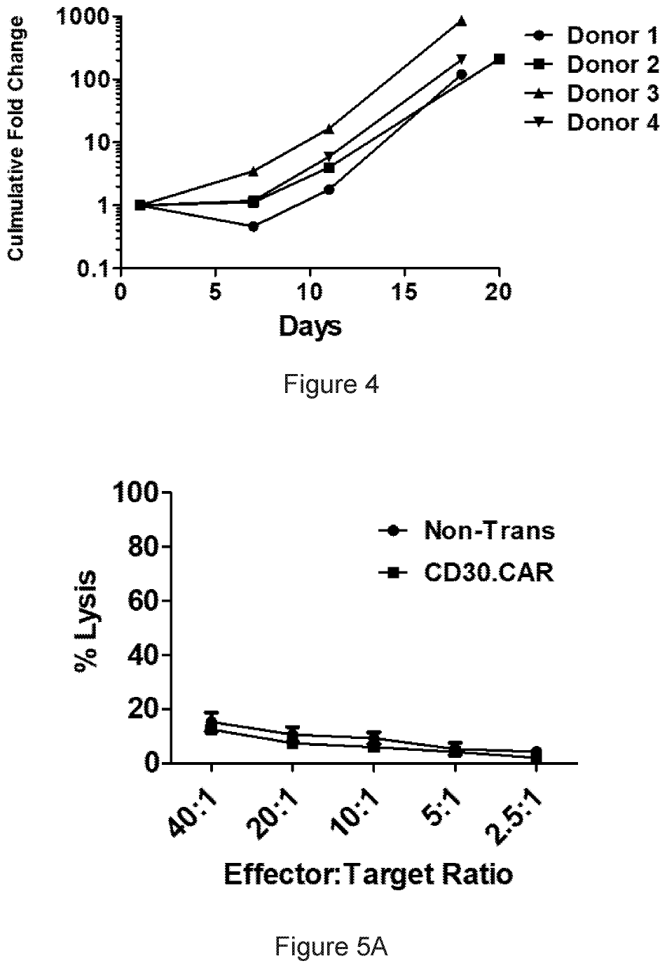

FIG. 4 shows that CD30.CAR EBVSTs produced from the 4 different healthy donor subjects expanded well in in vitro culture, sufficient to attain therapeutic doses of CD30.CAR EBVSTs within ˜17-20 days. The expanded cells expressed the CD30.CAR on 77% to 99% of cells (data not shown).

Analysis of CD30.CAR EBVST Cytotoxicity

The cytotoxic specificity of the CD30.CAR EBVSTs was measured using a chromium-51 (⁵¹Cr) release assay. Briefly, target cells, either CD30-negative BJAB Burkitt lymphoma cells or CD30-positive HDLM2 Hodgkin lymphoma cells were incubated with ⁵¹Cr for one hour. Non-transduced EBVSTs or CD30.CAR-transduced EBVSTs were used as effectors and were incubated with targets at effector-to-target ratios of 40:1, 20:1, 10:1, 5:1 and 2.5:1 in wells of 96-well plates. After 4-6 hours of incubation, coculture supernatants were harvested, and ⁵¹Cr release was detected with a gamma counter. The percentage of specific lysis was determined from the mean of triplicates using the following formula: [(experimental release−spontaneous release)/(maximum-release−spontaneous release)]×100.

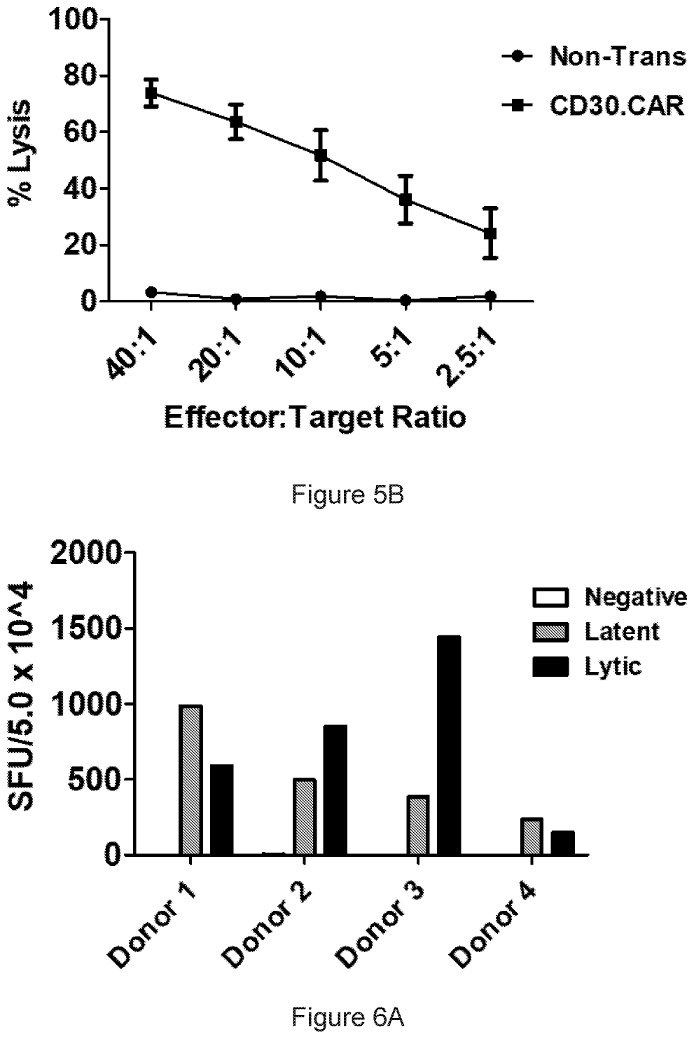

FIG. 5 shows that the CD30.CAR EBVSTs were substantially non-cytotoxic to cells of the CD30-negative Burkitt lymphoma BJAB cell line, but displayed high cytotoxicity to cells of the CD30-positive Hodgkin lymphoma HDLM2 cell line.

Analysis of Reactivity of CD30.CAR EBVSTs to EBV Antigens

IFN-γ ELISpot analysis was performed to evaluate the responses of CD30.CAR EBVSTs prepared from four different healthy donor subjects to stimulation with EBV antigens.

IFN-γ production was measured in response to stimulation with pepmixes (obtained from JPT Technologies, Berlin, Germany) for EBV latent cycle antigens (EBNA1, LMP1, LMP2 and BARF1) and EBV lytic cycle antigens (BZLF1, BRLF1, BMLF1, BMRF1, BMRF2, BALF2, BNLF2a and BNLF2b). Briefly, CD30.CAR EBVSTs were plated at 5×10⁴ cells/well in duplicate in wells of 96-well MultiScreen plates (MilliporeSigma). Stimulations were performed using a total of 0.1 μg peptide per well. After 16-20 hours of incubation at 37° C. in 5% CO₂, the plates were developed for IFN-γ+ spots and sent to ZellNet Consulting (Fort Lee, N.J.) for quantification. The frequency of antigen specific responses are expressed as spot forming units (SFU) per 5×10⁴ cells.

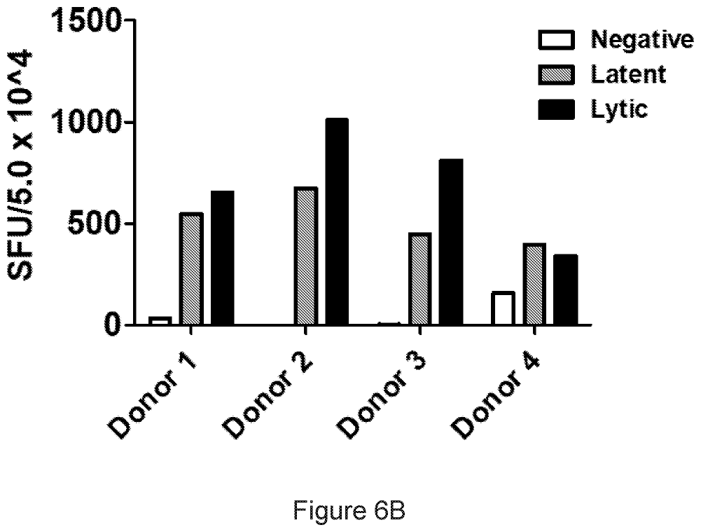

FIG. 6 shows that CD30.CAR EBVSTs produced from the 4 different healthy donor subjects retained their specificity for EBV antigens.

All four CD30.CAR EBVSTs lines passed the functional release criteria of having producing greater than 100 IFNγ spot-forming units (SFU) per 10⁵ cells in response to stimulation with both latent and lytic EBV antigens, and greater than 20% specific cytolysis against the CD30-positive Hodgkin lymphoma cell line, HDLM2, at an effector to target ratio of 20:1.

### 5.2 Administration of CD30.CAR EBVSTs as Allogeneic Adoptive Cell Therapy for CD30+ Lymphoma

Patients aged 12-75 years having CD30+ refractory or relapsed Hodgkin lymphoma, Non-Hodgkin lymphoma, ALK-positive anaplastic T cell lymphoma, ALK-negative anaplastic T cell lymphoma or other peripheral T-cell lymphoma were eligible for treatment in this study.

Patients received three daily doses of cyclophosphamide (Cy: 500 mg/m²/day) together with fludarabine (Flu: 30 mg/m²/day) to induce lymphopenia, completed at least 48 hours before CD30.CAR EBVST cell infusion, but no later than 2 weeks prior to infusion.

On Day 0 of study, patients received their planned single dose of allogeneic CD30.CAR EBVSTs by intravenous infusion over approximately 1 to 10 minutes, in a volume of 1 to 50 ml. Patients were administered with CD30.CAR EBVSTs having the best HLA class I and class II match.

A total of five patients were administered allogeneic CD30.CAR EBVST cells in the present study. Three patients received dose level 1 (DL1), of 4×10⁷ CD30.CAR EBVST cells. Two patients received dose level 2 (DL2), of 1×10⁸ V CD30.CAR EBVST cells.

Monitoring was undertaken according to institutional standards for administration of blood products, with the exception that the injection was given by a physician. Patients were monitored for at least 3 hours post infusion. Patients were assessed for adverse events, including changes in clinical status and laboratory data. In particular, patients were evaluated for correlates of cytokine release syndrome (CRS) and neurotoxicity, which have been observed in some CAR-T cell immunotherapies.

Blood samples were collected from patients at the following time points: pre study, 3-4 hours post infusion, 1, 2, 3, 4, and 6 weeks and 3 months post day 0 cell infusion. Samples were analysed in order to assess persistence and efficacy of CD30.CAR EBVSTs.

None of the patients experienced dose-limiting toxicities, and no cytokine release syndrome (CRS) or graft-vs-host disease (GVHD) of any grade was observed.

Clinical Responses in Patients Administered Allogeneic CD30.CAR EBVSTs

Diagnostic imaging was performed to document measurable disease and response to therapy (through PET scans, CT scans, MRI and nuclear imaging) pre-infusion and at 6-8 weeks following day 0 infusion.

Patient #1 was injected intravenously with 11.9 mCi of FDG in the left antecubital fossa (blood glucose level at the time of injection was 99 mg/dL). PET and CT images were obtained from the midcalvarium to proximal femora, and the images were subsequently fused, with multiplanar reconstruction in the axial, coronal and sagittal planes along with three-dimensional reconstructions.

Patient #2 was injected intravenously with 7.29 mCi of FDG (blood glucose level at the time of injection was 99 mg/dL). Approximately 60 min later, images from the skull base to the proximal thighs were acquired using a PET-CT scanner utilizing CT attenuation correction techniques. CT slices were obtained using the low-dose technique, and multiplanar reformatted images were obtained.

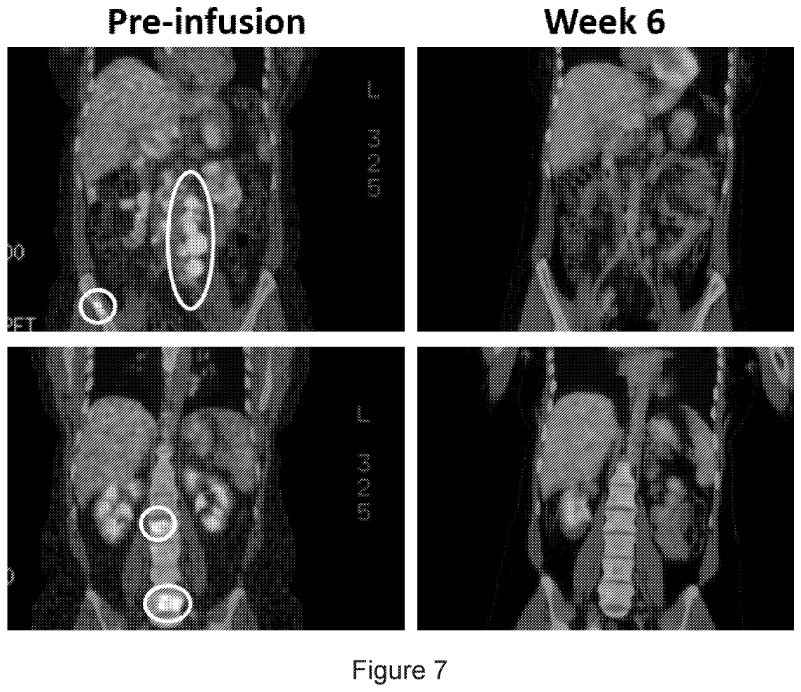

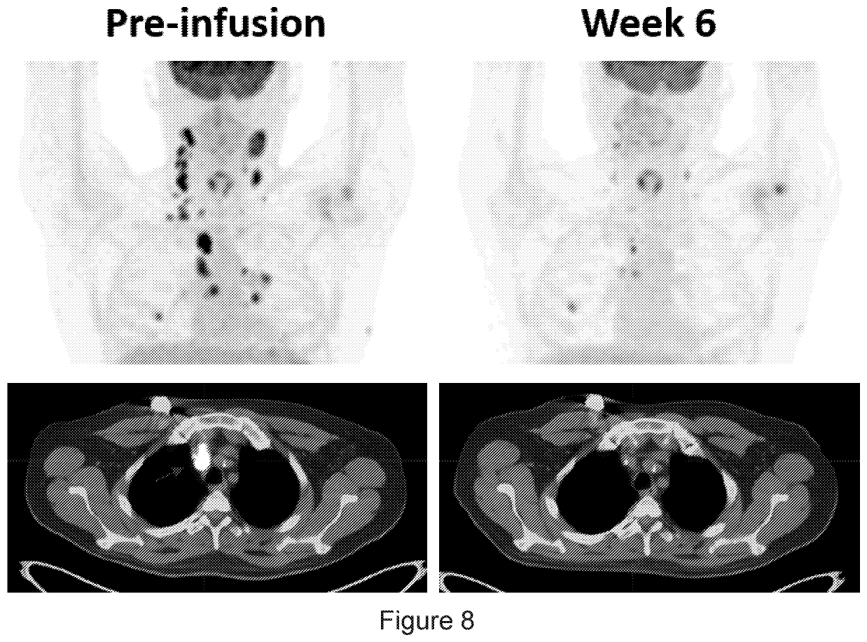

FIGS. 7 and 8 show clinical responses in two patients treated with CD30.CAR EBVSTs. Images for patient #1 show resolution of several areas of disease and images for patient #2 show a marked reduction of disease, indicative of therapeutic efficacy for treatment with allogeneic CD30.CAR EBVSTs in these patients.

Analysis of CD30.CAR Vector Copy Number Post-Administration

Integrated genome of the retrovirus encoding the CD30.CAR was quantified by real-time qPCR. PBMCs were isolated from peripheral blood samples taken from patients at several time points (Pre-lymphodepletion, 3 hrs, Week 1, Week 2, Week 3, Week 4, Week 6, and Month 3). After extracting DNA from PBMCs with the QIAamp DNA Blood Mini Kit (Qiagen) in accordance with the manufacturer's instructions, we amplified the DNA with primers and probes (Applied Biosystems) complementary to specific sequences within the retroviral vector. A standard curve was established using serial dilutions of the plasmid encoding the transgene. Amplifications were performed using the ABI7900HF Real-Time PCR System (Applied Biosystems) according to the manufacturer's instructions.

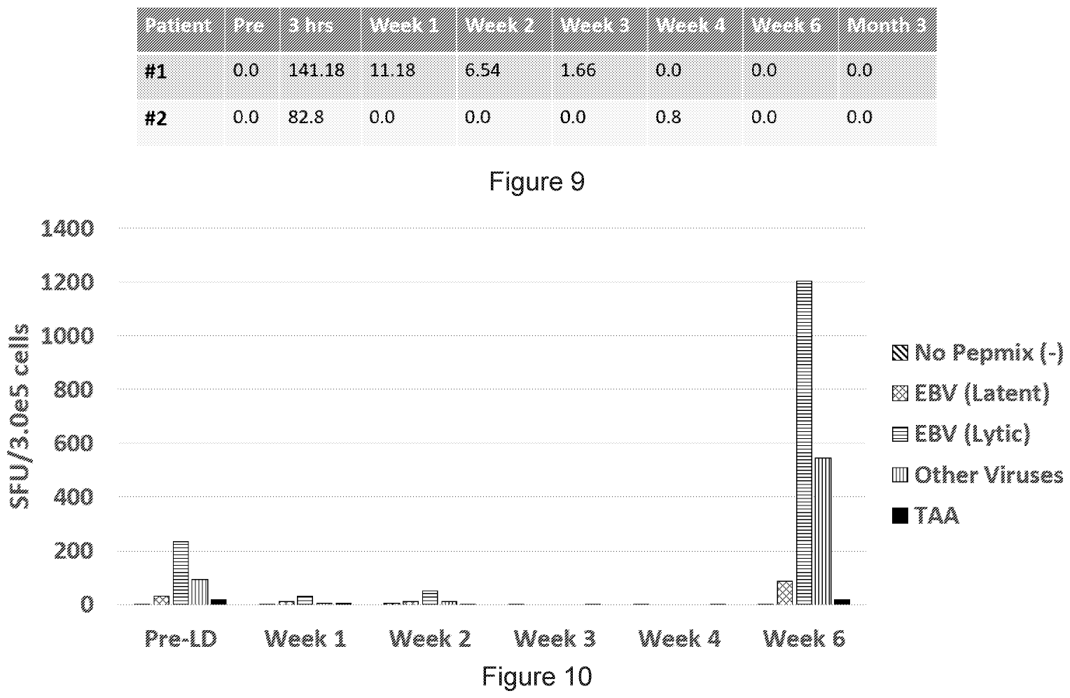

FIG. 9 shows vector copy number of the CD30.CAR transgene for patient #1 and patient #2, and suggest that CD30.CAR EBVSTs do not expand in vivo, and quickly become undetectable in the peripheral blood in these patients.

Analysis of Epitope Spreading in Patients Administered Allogeneic CD30.CAR EBVSTs

In order to evaluate epitope spreading, immune cells were collected from patient #1 at several time points, and stimulated with tumor-associated antigens to determine their reactivity before and after infusion of allogeneic CD30.CAR EBVSTs.

PBMCs were isolated from peripheral blood samples taken from patients at several time points (Pre-lymphodepletion, 3 hrs, Week 1, Week 2, Week 3, Week 4, Week 6, and Month 3) and used in an ELISpot assay performed essentially as described in Example 5.1 above, with the exception that PBMCs were plated at 3×10⁵ per well, and that in addition to evaluation of EBV latent and lytic antigens, two additional groups of antigens were used to stimulate PBMCs; (1) a pool of pepmixes of antigens from “Other Viruses” (adenovirus proteins Hexon and Penton, and CMV protein PP65), and (2) a pool of pepmixes corresponding to the tumor-associated antigens (TAA) MAGE-A4, NY-ESO, PRAME, SSX2, and Survivin.

FIG. 10 shows that Patient #1 did not have responses to the tumor-associated antigens at any time point, suggesting that there was no epitope spreading in patient #1. This result suggests that treatment with allogeneic CD30.CAR EBVSTs did not sensitize the patient's immunes system toward these other tumor antigens.

### 5.3 Conclusions

The inventors have shown that CD30.CAR EBVSTs produced from healthy donor subjects can be expanded to sufficient numbers and preserve the function of both their TCR and the CD30.CAR, with retention of EBV specificity and the ability to eliminate CD30-positive tumor cells, in accordance with their use as an off-the-shelf treatment for patients with CD30+ cancer.

CD30.CAR EBVSTs were found to be safe, and to display therapeutic efficacy against CD30-positive lymphoma in vivo in allogeneic recipients. Clinical responses were observed despite the limited persistence of CAR-expressing cells in the peripheral blood, and in the absence of evidence of epitope spreading to other tumor-associated antigens.

1. A virus-specific immune cell comprising a chimeric antigen receptor (CAR) or nucleic acid encoding a CAR for use in a method of treating or preventing a disease or condition characterised by an alloreactive immune response, wherein the CAR comprises: (i) an antigen-binding domain which binds specifically to CD30, (ii) a transmembrane domain, and (iii) a signalling domain, wherein the signalling domain comprises: (a) an amino acid sequence derived from the intracellular domain of CD28, and (b) an amino acid sequence comprising an immunoreceptor tyrosine-based activation motif (ITAM).

2. Use of a virus-specific immune cell comprising a chimeric antigen receptor (CAR) or nucleic acid encoding a CAR in the manufacture of a medicament for use in treating or preventing a disease or condition characterised by an alloreactive immune response, wherein the CAR comprises: (i) an antigen-binding domain which binds specifically to CD30, (i) a transmembrane domain, and (iii) a signalling domain, wherein the signalling domain comprises: (a) an amino acid sequence derived from the intracellular domain of CD28, and (b) an amino acid sequence comprising an immunoreceptor tyrosine-based activation motif (ITAM).

3. A method of treating or preventing a disease or condition characterised by an alloreactive immune response, comprising administering to a subject a therapeutically or prophylactically effective quantity of virus-specific immune cells comprising a chimeric antigen receptor (CAR) or nucleic acid encoding a CAR, wherein the CAR comprises: (i) an antigen-binding domain which binds specifically to CD30, (ii) a transmembrane domain, and (iii) a signalling domain, wherein the signalling domain comprises: (a) an amino acid sequence derived from the intracellular domain of CD28, and (b) an amino acid sequence comprising an immunoreceptor tyrosine-based activation motif (ITAM).

4. The virus-specific immune cell for use according to claim 1, the use according to claim 2, or the method according to claim 3, wherein the disease or condition characterised by an alloreactive immune response is a disease or condition associated with allotransplantation.

5. The virus-specific immune cell for use, the use, or the method according to any one of claims 1 to 4, wherein the disease or condition is graft versus host disease (GVHD).

6. The virus-specific immune cell for use, the use, or the method according to any one of claims 1 to 4, wherein the disease or condition is graft rejection.

7. The virus-specific immune cell for use, the use, or the method according to any one of claims 1 to 6, wherein the method comprises administering a therapeutically or prophylactically effective quantity of the virus-specific immune cells to a donor subject for an allotransplant prior to harvesting the allotransplant.

8. The virus-specific immune cell for use, the use, or the method according to any one of claims 1 to 7, wherein the method comprises administering a therapeutically or prophylactically effective quantity of the virus-specific immune cells to a recipient subject for an allotransplant.

9. The virus-specific immune cell for use, the use, or the method according to any one of claims 1 to 8, wherein the method comprises contacting an allotransplant with a therapeutically or prophylactically effective quantity of the virus-specific immune cells.

10. A virus-specific immune cell comprising a chimeric antigen receptor (CAR) or nucleic acid encoding a CAR for use in a method of treating or preventing a disease or condition by allotransplantation, wherein the CAR comprises: (i) an antigen-binding domain which binds specifically to CD30, (ii) a transmembrane domain, and (iii) a signalling domain, wherein the signalling domain comprises: (a) an amino acid sequence derived from the intracellular domain of CD28, and (b) an amino acid sequence comprising an immunoreceptortyrosine-based activation motif (ITAM).

11. Use of a virus-specific immune cell comprising a chimeric antigen receptor (CAR) or nucleic acid encoding a CAR in the manufacture of a medicament for use in treating or preventing a disease or condition by allotransplantation, wherein the CAR comprises: (i) an antigen-binding domain which binds specifically to CD30, (ii) a transmembrane domain, and (iii) a signalling domain, wherein the signalling domain comprises: (a) an amino acid sequence derived from the intracellular domain of CD28, and (b) an amino acid sequence comprising an immunoreceptor tyrosine-based activation motif (ITAM).

12. A method of treating or preventing a disease or condition by allotransplantation, comprising administering to a subject a therapeutically or prophylactically effective quantity of virus-specific immune cells comprising a chimeric antigen receptor (CAR) or nucleic acid encoding a CAR, wherein the CAR comprises: (i) an antigen-binding domain which binds specifically to CD30, (ii) a transmembrane domain, and (ii) a signalling domain, wherein the signalling domain comprises: (a) an amino acid sequence derived from the intracellular domain of CD28, and (b) an amino acid sequence comprising an immunoreceptortyrosine-based activation motif (ITAM).

13. The virus-specific immune cell for use according to claim 10, the use according to claim 11, or the method according to claim 12, wherein the method comprises administering a therapeutically or prophylactically effective quantity of the virus-specific immune cells to a donor subject for an allotransplant prior to harvesting the allotransplant.

14. The virus-specific immune cell for use, the use, or the method according to any one of claims 10 to 13, wherein the method comprises administering a therapeutically or prophylactically effective quantity of the virus-specific immune cells to a recipient subject for an allotransplant.

15. The virus-specific immune cell for use, the use, or the method according to any one of claims 10 to 14, wherein the method comprises contacting an allotransplant with a therapeutically or prophylactically effective quantity of the virus-specific immune cells.

16. The virus-specific immune cell for use, the use, or the method according to any one of claims 10 to 15, wherein the allotransplantation comprises adoptive transfer of allogeneic immune cells.

17. The virus-specific immune cell for use, the use, or the method according to any one of claims 10 to 16, wherein the disease or condition is a T cell dysfunctional disorder, a cancer or an infectious disease.

18. A method of killing alloreactive immune cells, comprising contacting alloreactive immune cells with a virus-specific immune cell comprising a chimeric antigen receptor (CAR) or nucleic acid encoding a CAR for use in a method of treating or preventing a disease or condition characterised by an alloreactive immune response, wherein the CAR comprises: (i) an antigen-binding domain which binds specifically to CD30, (ii) a transmembrane domain, and (iii) a signalling domain, wherein the signalling domain comprises: (a) an amino acid sequence derived from the intracellular domain of CD28, and (b) an amino acid sequence comprising an immunoreceptor tyrosine-based activation motif (ITAM).

19. The virus-specific immune cell for use, the use, or the method according to any one of claims 1 to 18, wherein the signalling domain comprises an amino acid sequence having at least 80% amino acid sequence identity to SEQ ID NO:26.

20. The virus-specific immune cell for use, the use, or the method according to any one of claims 1 to 19, wherein the transmembrane domain is derived from the transmembrane domain of CD28.

21. The virus-specific immune cell for use, the use, or the method according to any one of claims 1 to 20, wherein the transmembrane domain comprises an amino acid sequence having at least 80% amino acid sequence identity to SEQ ID NO:20.

22. The virus-specific immune cell for use, the use, or the method according to any one of claims 1 to 21, wherein the antigen-binding domain comprises an amino acid sequence having at least 80% amino acid sequence identity to SEQ ID NO:14, and an amino acid sequence having at least 80% amino acid sequence identity to SEQ ID NO:15.

23. The virus-specific immune cell for use, the use, or the method according to any one of claims 1 to 22, wherein the antigen-binding domain comprises an amino acid sequence having at least 80% amino acid sequence identity to SEQ ID NO:18.

24. The virus-specific immune cell for use, the use, or the method according to any one of claims 1 to 23, wherein the signalling domain comprises: (a) an amino acid sequence derived from the intracellular domain of CD3ζ.

25. The virus-specific immune cell for use, the use, or the method according to any one of claims 1 to 24, wherein the signalling domain comprises an amino acid sequence having at least 80% amino acid sequence identity to SEQ ID NO:25.

26. The virus-specific immune cell for use, the use, or the method according to any one of claims 1 to 25, wherein the CAR additionally comprises a hinge region provided between the antigen-binding domain and the transmembrane domain.

27. The virus-specific immune cell for use, the use, or the method according to claim 26, wherein the hinge region comprises an amino acid sequence having at least 80% amino acid sequence identity to SEQ ID NO:33.

28. The virus-specific immune cell for use, the use, or the method according to any one of claims 1 to 27, wherein the CAR comprises an amino acid sequence having at least 80% amino acid sequence identity to SEQ ID NO:35 or 36.

29. The virus-specific immune cell for use, the use, or the method according to any one of claims 1 to 28, wherein the virus-specific immune cell comprises a CAR comprising an antigen-binding domain which binds specifically to a target antigen other than CD30, or nucleic acid encoding a CAR comprising an antigen-binding domain which binds specifically to a target antigen other than CD30.

30. The virus-specific immune cell for use, the use, or the method according to any one of claims 1 to 29, wherein:
the CAR comprises: (i) an antigen-binding domain which binds specifically to CD30, (ii) a transmembrane domain, and (iii) a signalling domain comprising an immunoreceptor tyrosine-based activation motif (ITAM); and
wherein the virus-specific immune cell comprises a CAR comprising an antigen-binding domain which binds specifically to a target antigen other than CD30, or nucleic acid encoding a CAR comprising an antigen-binding domain which binds specifically to a target antigen other than CD30.

31. The virus-specific immune cell for use, the use, or the method according to claim 29 or claim 30, wherein the virus-specific immune cell comprises more than one non-identical CAR, or nucleic acid encoding more than one non-identical CAR.

32. The virus-specific immune cell for use, the use, or the method according to any one of claims 29 to 31, wherein the target antigen other than CD30 is a cancer cell antigen.

33. The virus-specific immune cell for use, the use, or the method according to any one of claims 29 to 32, wherein the target antigen other than CD30 is selected from CD19, CD20, CD22, ROR1R, CD4, CD7, CD38, BCMA, Mesothelin, EGFR, GPC3, MUC1, HER2, GD2, CEA, EpCAM, LeY and PSCA.

34. The virus-specific immune cell for use, the use, or the method according to any one of claims 29 to 33, wherein the target antigen other than CD30 is CD19.

35. The virus-specific immune cell for use, the use, or the method according to any one of claims 1 to 34, wherein the virus-specific immune cell is a virus-specific T cell.

36. The virus-specific immune cell for use, the use, or the method according to any one of claims 1 to 35, wherein the virus-specific immune cell is specific for Epstein-Barr virus (EBV).
